(19) United States
(12) Patent Application Publication
(10) Pub. No.: US 2026/0226440 A1
(43) Pub. Date: Aug. 6, 2026

(54) GENE EDITING SYSTEMS COMPRISING TYPE V CRISPR NUCLEASE AND ENGINEERED GUIDE RNA

(71) Applicant: ARBOR BIOTECHNOLOGIES, INC., Cambridge, MA (US)

(72) Inventors: Noah Michael JAKIMO, Cambridge, MA (US); Colin Alexander MCGAW, Cambridge, MA (US); Alexander THOMAS, Cambridge, MA (US); Chad David TORGERSON, Cambridge, MA (US); Roy ZIBLAT, Cambridge, MA (US)

(21) Appl. No.: 19/152,307

(22) PCT Filed: Jan. 31, 2024
(86) PCT No.: PCT/US2024/013723

§ 371 Date: Jul. 30, 2025

Related U.S. Application Data
(60) Provisional application No. 63/442,679, filed on Feb. 1, 2023.

(51) Int. Cl. C12N 9/22; C12N 9/12; C12N 15/11; C12N 15/90

(52) U.S. Cl. CPC C12N 9/226; C12N 9/1276; C12N 15/11; C12N 15/907; C12Y 207/07049; C07K 2319/00; C12N 2310/20

(57) ABSTRACT

A gene editing system comprising: (a) a Type V CRISPR nuclease polypeptide or a first nucleic acid encoding the Type V CRISPR nuclease polypeptide; (b) an engineered guide RNA (gRNA) or a second nucleic acid encoding the gRNA, wherein the engineered gRNA comprises: (i) a spacer sequence specific to a target sequence within a genomic site of interest, and (ii) an engineered scaffold sequence, which is recognizable by the Type V CRISPR nuclease, wherein the engineered scaffold sequence comprises one or more mutations relative to the wild-type counterpart.

### CROSS REFERENCE TO RELATED APPLICATIONS

This application claims the benefit under 35 U.S.C. § 119(e) of U.S. Provisional Application No. 63/442,679, filed Feb. 1, 2023, the content of which is incorporated by reference herein in its entirety.

### SEQUENCE LISTING

The instant application contains a Sequence Listing which has been filed electronically in XML format and is hereby incorporated by reference in its entirety. Said XML copy, created on Jan. 29, 2024, is named 063586-510001WO_SeqList_ST26.xml and is 131 kilobytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

The present disclosure is based, at least in part, on the development of engineered scaffold sequences for use in guide RNAs recognizable by a Type V CRISPR nuclease. Gene editing systems comprising the Type V CRISPR nuclease and guide RNAs (gRNAs) comprising such engineered scaffold sequences show enhanced editing efficiencies relative to gRNAs comprising the parent scaffold sequence (e.g., SEQ ID NO: 71) at various genomic sites. Further, the present disclosure provides variants of the Type V CRISPR nuclease, which exhibited enhanced gene editing efficiency, e.g., when fused with a reverse transcriptase (RT).

Accordingly, provided herein are gene editing systems involving gRNAs having engineered scaffold sequences as disclosed herein and the Type V CRISPR nuclease or a variant thereof as also disclosed herein. Such a gene editing system may further comprise an RT, which may form a fusion polypeptide with the Type V CRISPR nuclease in some instances, and a reverse transcription donor RNA, which may form a fusion polynucleotide with the gRNA in some instances. Also provided herein are gene editing methods using any of the gene editing systems disclosed herein. Components of the gene editing systems disclosed herein, e.g., gRNAs comprising an engineered scaffold sequence or Type V CRISPR nuclease-RT fusion polypeptides, are also within the scope of the present disclosure.

In some aspects, the present disclosure features a gene editing system comprising: (a) a polypeptide comprising a Type V CRISPR nuclease or a first nucleic acid encoding the polypeptide; wherein the Type V CRISPR nuclease comprises an amino acid at least 95% identical to SEQ ID NO: 1; and (b) an engineered guide RNA (gRNA) or a second nucleic acid encoding the engineered gRNA. The engineered gRNA comprises: (i) a spacer sequence specific to a target sequence within a genomic site of interest and (ii) an engineered scaffold sequence, which is recognizable by the Type V CRISPR nuclease. The engineered scaffold sequence comprises one or more mutations relative to the parent scaffold sequence, which comprises the nucleotide sequence of SEQ ID NO: 71. In some instances, the engineered scaffold is at least 80% identical to SEQ ID NO: 71. In some instances, the spacer sequence is located at the 3′ of the scaffold sequence. In some instances, the engineered scaffold sequence may be about 115-135 nucleotides in length.

In some embodiments, the engineered scaffold sequence comprises one or more of the following mutations relative to the wide-type counterpart: (i) nucleotide substitution at one or more of positions 25, 26, 30, 38, 48, 52, 55, 66, 67, 79, 82-87, 91-93, 96, 98-100, 104, 105, 107, 110, 113, 115, 118-121, 123 of SEQ ID NO: 71; (ii) one or more deletions at positions of 25-31, 52-55, and 84-87 of SEQ ID NO: 71; and (iii) one or more mutation within positions 72-77 of SEQ ID NO: 71; wherein the one or more mutations comprise nucleotide substitutions, deletions, insertions, or a combination thereof. In some instances, the engineered scaffold sequence may further comprise: (iv) an insertion at 5′ end, between positions 107 and 108, and/or between positions 114 and 115. In some examples, the engineered scaffold sequence is selected from those listed in Table 2 and Table 4. In specific examples, the engineered scaffold sequence is one of C9, C10, C11, and C16 (identified in Table 4).

In some embodiments, the gene editing system disclosed herein may further comprise: (c) a reverse transcriptase (RT) or a third nucleic acid encoding the RT; and (d) a reverse transcription donor RNA (RT donor RNA) or a fourth nucleic acid encoding the RT donor RNA, wherein the RT donor RNA comprises a primer binding site (PBS) and a template sequence. Exemplary RT enzymes include, but are not limited to, Moloney Murine Leukemia Virus (MMLV)-RT, mouse mammary tumor virus (MMTV)-RT, Marathon-RT, and RTx-RT.

In some embodiments, the Type V CRISPR nuclease and the RT of the gene editing system form a fusion polypeptide. In some instances, the fusion polypeptide comprises one or more mutations in the Type V CRISPR nuclease relative to the wild-type counterpart. Such a fusion polypeptide exhibits enhanced editing activity relative to the wild-type counterpart. In some examples, the fusion polypeptide comprises the one or more amino acid substitutions depicted in FIG. 5. In some specific examples, the fusion polypeptide comprises S136G, A138R, D137G, and/or D137R substitutions in the Type V CRISPR nuclease relative to SEQ ID NO: 1.

In some embodiments, the gene editing system provided herein comprises: (a) a fusion polypeptide comprising a Type V CRISPR nuclease and a reverse transcriptase (RT), or a first nucleic acid encoding the fusion polypeptide; (b) an engineered guide RNA (gRNA) or a second nucleotide encoding the engineered gRNA; and (c) a reverse transcription donor RNA (RT donor RNA) or a fourth nucleic acid encoding the RT donor RNA. The RT donor RNA comprises a primer binding site (PBS) and a template sequence. The fusion polypeptide comprises one or more mutations in the Type V CRISPR nuclease relative to the wild-type counterpart. Such a fusion polypeptide exhibits enhanced editing activity relative to the wild-type counterpart. In some examples, the fusion polypeptide comprises the one or more amino acid substitutions depicted in FIG. 5, e.g., comprises S136G, A138R, D137G, and/or D137R substitutions in the Type V CRISPR nuclease relative to SEQ ID NO: 1.

In some embodiments, any of the gene editing systems disclosed herein may comprise the Type V CRISPR nuclease, the RT, or a fusion polypeptide of the Type V CRISPR nuclease and the RT. Alternatively, the gene editing system may comprise at least one nucleic acid that expresses the Type V CRISPR nuclease and/or the RT. For example, the gene editing system may comprise a nucleic acid that expresses a fusion polypeptide comprising the Type V CRISPR and the RT. In some instances, the at least one nucleic acid is a vector, which optionally is a viral vector. Alternatively, the at least one nucleic acid is a messenger RNA.

In some examples, the PBS in the RT donor RNA can be about 10-60-nucleotide in length, for example, about 20-40-nucleotide in length (e.g., about 30-nucleotide in length). In some instances, the PBS binds a PBS-targeting site that is adjacent to the complementary region of the target sequence, and wherein the PBS-targeting site is upstream to the complementary region of the target sequence. In some examples, the template sequence is about 5-100-nucleotide in length, for example, about 30-50-nucleotide in length (e.g., about 45-nucleotide in length). In some instances, the template sequence is homologous to the genomic site of interest and comprises one or more nucleotide variations relative to the genomic site of interest.

In some instances, the engineered gRNA and the RT donor RNA of a gene editing system disclosed herein may be located on a single RNA molecule. In some examples, the engineered gRNA and the RT donor RNA are connected via a nucleotide linker (e.g., a polyA nucleotide linker such as an A5 linker).

In some instances, the gene editing system disclosed herein comprises one or more lipid nanoparticles (LNPs), which are associated with one or more of elements (a)-(d) of the system. In some examples, the one or more LNPs are associated with up to three elements of (a)-(d), and wherein the system comprises at least one vector that expresses the remaining element(s).

Further, the present disclosure provides a pharmaceutical composition comprising any of the gene editing systems disclosed herein, as well as a kit comprising the elements of the gene editing system.

In other aspects, the present disclosure provides a method for genetically editing a cell, the method comprising contacting a host cell the gene editing system disclosed herein or the pharmaceutical composition comprising such to genetically edit the host cell. In some instances, the host cell is cultured in vitro. In other instances, the contacting step is performed by administering the gene editing system to a subject comprising the host cell.

Also with the scope of the present disclosure are components of any of the gene editing systems disclosed herein.

In some aspects, the present disclosure features a polynucleotide, comprising (a) any of the engineered guide RNA (gRNA), and optionally (b) a reverse transcriptase template (RTT) RNA, which optionally is located upstream to (a). The engineered gRNA comprises a spacer sequence and any of the engineered scaffold sequences as disclosed herein. The polynucleotide may further comprise a nucleotide linker between (a) and (b). In some instances, the nucleotide linker is a polyA linker (e.g., an A5 linker). In addition, the polynucleotide may further comprise a 5′ end U6 start fragment, an end protection fragment, a 3′ end U6 termination fragment, or a combination thereof.

In other aspects, provided herein is a fusion polypeptide comprising a Type V CRISPR nuclease and a reverse transcriptase (RT). The fusion polypeptide comprises one or more mutations in the Type V CRISPR nuclease portion relative to the wild-type counterpart as disclosed herein and exhibits enhanced editing activity relative to the wild-type counterpart. Nucleic acids (e.g., vectors) encoding such a fusion polypeptide and host cells comprising the nucleic acids are also within the scope of the present disclosure.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1D illustrate indel activity of various engineered sgRNAs relative to the parent sgRNA. FIG. 1A: indel activity of StemLoopTrunc_1-14 relative to the parent sgRNA. FIG. 1B: indel activity of StemLoopTrunc_15-24 relative to parent sgRNA. FIG. 1C: indel activity of StemLoopTrunc_25-36 relative to the parent sgRNA. FIG. 1D: indel activity of StemPairing_3-20 relative to parent sgRNA. *: P≤0.05. **: P≤0.01. ***: P≤0.001.

FIGS. 2A-2B illustrate that nuclease-RT fusion enables precise editing on EMX1 locus (FIG. 2A) and on VEGFA locus (FIG. 2B) in HEK293T cells.

FIG. 3 illustrates percentage of NGS reads comprising indels or precise edits at an EMX1 target following transfection of variant nuclease-RT fusion polypeptides in HEK293T cells.

FIG. 4 illustrates percentage of NGS reads comprising indels or precise edits at a VEGFA target following transfection of variant nuclease-RT fusion polypeptides in HEK293T cells.

FIG. 5 illustrates variants exhibiting enhanced precision editing compared to wild-type nuclease RT fusion polypeptide.

### DETAILED DESCRIPTION

The present disclosure relates to gene editing systems that exhibit enhanced gene editing efficiencies. In some embodiments, the gene editing system disclosed herein comprises a Type V CRISPR nuclease or a nucleic acid encoding such, and an engineered guide RNA (gRNA) comprising a spacer sequence and an engineered scaffold sequence, which comprises one or more mutations relative to the parent scaffold sequence (e.g., SEQ ID NO: 71 disclosed herein). Such a gene editing system may further comprise a reverse-transcriptase (RT) and a reverse transcription donor RNA. The RT may form a fusion polypeptide with the Type V CRISPR nuclease. Alternatively or in addition, the reverse transcription donor RNA and the engineered gRNA may form a single polynucleotide (e.g., an editing template RNA as disclosed herein). In other embodiments, the gene editing system disclosed herein comprises a fusion polypeptide of a Type V CRISPR nuclease and an RT, a gRNA, and a reverse transcription donor RNA. The Type V CRISPR nuclease portion in the fusion polypeptide may comprise one or more mutations relative to the wild-type counterpart (e.g., SEQ ID NO:1 disclosed herein). In some instances, the gRNA and the reverse transcription donor RNA may form a single polynucleotide (e.g., an editing template RNA as disclosed herein). Any of the gene editing systems may be used to genetically edit a genomic site of interest, e.g., introducing mutations at the genomic site of interest via reverse transcription. Any of the components contained in the gene editing systems disclosed herein, e.g., engineered gRNAs, editing template RNAs comprising such, and Type V CRISPR nuclease-RT fusion polypeptides or encoding nucleic acids thereof, are also with the scope of the present disclosure.

### Definitions

The present disclosure will be described with respect to particular embodiments and with reference to certain FIGS., but the disclosure is not limited thereto but only by the claims. Terms as set forth hereinafter are generally to be understood in their common sense unless indicated otherwise.

As used herein, the term “activity” refers to a biological activity. In some embodiments, the activity refers to effector activity. In some embodiments, activity includes enzymatic activity, e.g., catalytic ability of an effector. For example, activity can include nuclease activity. In another example, activity refers to the ability of an enzyme to generate DNA from RNA or to introduce an edit into a target sequence.

As used herein, the term “CRISPR nuclease” refers to an RNA-guided effector that is capable of binding a nucleic acid and introducing a single-stranded break or double-stranded break. In some embodiments, a CRISPR nuclease is a Type II CRISPR nuclease or a Type V CRISPR nuclease. In some embodiments, a CRISPR nuclease is an effector as described in Makarova et al. “Classification and Nomenclature of CRISPR-Cas Systems: Where from Here?” CRISPRJ. 1(5):325-36 (2018).

As used herein, the terms “Type V” and “Type V nuclease” refer to an RNA-guided CRISPR nuclease with a RuvC domain. In some embodiments, a Type V nuclease does not require a tracrRNA. In some embodiments, a Type V nuclease requires a tracrRNA. In some embodiments, the Type V nuclease is a Cas12 polypeptide, e.g., comprising the amino acid sequence of SEQ ID NO:1 or a variant thereof as disclosed herein.

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, word length=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

As used herein, the terms “RNA guide”, “RNA guide sequence,” or “guide RNA (gRNA)” refer to an RNA molecule or a modified RNA molecule that facilitates the targeting of a CRISPR nuclease described herein to a genomic site of interest. For example, an RNA guide can be a molecule that An RNA guide comprises a spacer sequence and a scaffold sequence. The spacer sequence recognizes (e.g., binds to) a site in a non-PAM strand that is complementary to a target sequence in the PAM strand, e.g., designed to be complementary to a specific nucleic acid sequence. The scaffold sequence contains a nuclease binding sequence (e.g., a direct repeat (DR) sequence) for binding to the CRISPR nuclease. The terms CRISPR RNA (crRNA), pre-crRNA and mature crRNA are also used herein to refer to an RNA guide. In some instances, the 5′ end or 3′ end of an RNA guide may be fused to an RT donor RNA as disclosed herein. In some instances, the gRNA can be a modified RNA molecule comprising one or more deoxyribonucleotides, for example, in a DNA-binding sequence contained in the gRNA, which binds the complementary sequence of the target sequence. In some examples, the DNA-binding sequence may contain a DNA sequence or a DNA/RNA hybrid sequence.

As used herein, the term “spacer” and “spacer sequence” (a.k.a., a DNA-binding sequence) is a portion in an RNA guide that is the RNA equivalent of the target sequence (a DNA sequence). The spacer contains a sequence capable of binding to the non-PAM strand via base-pairing at the site complementary to the target sequence (in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference.

As used herein, the term “binding site recognizable by a nuclease” or “nuclease binding sequence” refers to a sequence that is capable of binding to a CRISPR nuclease. In some embodiments, the nuclease binding sequence is an RNA sequence. In some embodiments, the nuclease binding sequence is a direct repeat sequence. In some embodiments, a nuclease binding sequence is capable of binding to a Type V CRISPR nuclease (e.g., binding site recognizable by a Type V CRISPR nuclease).

As used herein, the term “protospacer adjacent motif” or “PAM sequence” refers to a DNA sequence adjacent to a target sequence. In some embodiments, a PAM sequence is required for enzyme activity. In a double-stranded DNA molecule, the strand containing the PAM motif is called the “PAM-strand” and the complementary strand is called the “non-PAM strand.” The gRNA binds to a site in the non-PAM strand that is complementary to a target sequence disclosed herein, and the PAM sequence as described herein is present in the PAM-strand.

As used herein, the term “PAM strand” refers to the strand of a target nucleic acid (double-stranded) that comprises a PAM motif. In some embodiments, the PAM strand is a coding (e.g., sense) strand. In other embodiments, the PAM strand is a non-coding (e.g., antisense strand). The term “non-PAM strand” refers to the complementary strand of the PAM strand. Since a gRNA binds the non-PAM strand via base-pairing, the non-PAM strand is also known as the target strand, while the PAM strand is also known as the non-target strand.

As used herein, the term “target sequence” refers to a DNA fragment adjacent to a PAM motif (on the PAM strand). The complementary region of the target sequence is on the non-PAM strand. A target sequence may be immediately adjacent to the PAM motif. Alternatively, the target sequence and the PAM may be separately by a small sequence segment (e.g., up to 5 nucleotides, for example, up to 4, 3, 2, or 1 nucleotide). A target sequence may be located at the 3′ end of the PAM motif or at the 5′ end of the PAM motif, depending upon the CRISPR nuclease that recognizes the PAM motif, which is known in the art. For example, a target sequence is located at the 3′ end of a PAM motif for a Cas12i polypeptide (e.g., a Cas12i2 polypeptide such as those disclosed herein).

As used herein, the term “complementary” refers to a first polynucleotide (e.g., a spacer sequence of an RNA guide) that has a certain level of complementarity to a second polynucleotide (e.g., the complementary sequence of a target sequence) such that the first and second polynucleotides can form a double-stranded complex via base-pairing to permit an effector polypeptide (e.g., a Cas12i2 polypeptide, a Cas12i2-reverse transcriptase fusion polypeptide, or a variant thereof) that is complexed with the first polynucleotide to act on (e.g., cleave) the second polynucleotide. In some embodiments, the first polynucleotide may be substantially complementary to the second polynucleotide, i.e., having at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second polynucleotide. In some embodiments, the first polynucleotide is completely complementary to the second polynucleotide, i.e., having 100% complementarity to the second polynucleotide.

As used herein, the terms “reverse transcriptase” and “RT” refer to a multi-functional enzyme that typically has three enzymatic activities including RNA- and DNA-dependent DNA polymerization activity and an RNase H activity that catalyzes the cleavage of RNA in RNA-DNA hybrids. A reverse transcriptase can generate DNA from an RNA template.

As used herein, the terms “reverse transcription donor RNA” and “RT donor RNA” refer to an RNA molecule comprising a reverse transcription template sequence (template sequence) and a primer binding site (PBS). An RT donor RNA may be fused to an RNA guide at either the 5′ end or 3′ end of the gRNA.

As used herein, the term “PBS-targeting site” refers to the region to which a PBS binds. The PBS-targeting site may be adjacent to (e.g., upstream to) a region of the non-PAM strand that is complementary to the target sequence. For example, the PBS-targeting site can be 3-10 nucleotides (e.g., 3-nucleotide or 4-nucleotide) upstream to the region that is complementary to the target sequence. In some instances, the PBS-targeting site may be immediately adjacent to the region of the non-PAM stand that is complementary to the target sequence. In other examples, the PBS-targeting site may overlap with the region of the non-PAM strand that is complementary to the target sequence. Alternatively, the PBS-targeting site may be adjacent to, upstream to, or overlap with the target sequence on the PAM strand.

As used herein, the term “reverse transcription template sequence” or “template sequence” refers to an RNA molecule or a fragment of an RT donor RNA that serves as a template for DNA synthesis by a reverse transcriptase. In some embodiments, the reverse transcription template sequence comprises an edit to be incorporated into a genomic site where gene editing is needed. In some instances, an edit mediated by the reverse transcription template sequence in the RT donor RNA disrupts or removes the PAM sequence, the target sequence, or both.

As used herein, the term “editing template RNA” or “gene editing RNA” (used herein interchangeably) refers to an RNA molecule or a set of RNA molecules comprising an RNA guide (comprising a spacer and one or more binding site recognizable by a CRISPR nuclease such as those disclosed herein) and a RT doner RNA (comprising a PBS and a reverse transcription template sequence). A gene editing RNA is capable of mediating cleavage at a target sequence within a genomic site of interest by a CRISPR nuclease and synthesis of a DNA fragment from a free 3′end of a free DNA strand generated by the CRISPR nuclease cleavage based on the template sequence in the gene editing RNA. In some embodiments, an editing template RNA or gene editing RNA is a single RNA molecule comprising the gRNA linked (e.g., fused) to the RT donor RNA. In some embodiments, an editing template RNA from 5′ to 3′ comprises one or more binding site recognizable by a CRISPR nuclease, a spacer sequence, a PBS, and an RT donor RNA. In some embodiments, an editing template RNA or gene editing RNA from 5′ to 3′ comprises one or more binding site recognizable by a CRISPR nuclease, a spacer, a template sequence, and a PBS. In some embodiments, an editing template RNA or gene editing RNA from 5′ to 3′ comprises a template sequence, a PBS, one or more binding site recognizable by a CRISPR nuclease, and a spacer sequence. In some embodiments, an editing template RNA further comprises a linker. For example, in some embodiments, an editing template RNA comprises a linker between the one or more binding site recognizable by a CRISPR nuclease and the PBS or between the spacer sequence and the RT donor RNA.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, a first sequence is adjacent to a second sequence if the two sequences are separated by up to 2 nucleotides, up to 5 nucleotides, up to 8 nucleotides, up to 10 nucleotides, up to 12 nucleotides, or up to 15 nucleotides. In some embodiments, a first sequence is adjacent to a second sequence if the two sequences are separated by 2-5 nucleotides, 4-6 nucleotides, 4-8 nucleotides, 4-10 nucleotides, 6-8 nucleotides, 6-10 nucleotides, 6-12 nucleotides, 8-10 nucleotides, 8-12 nucleotides, 10-12 nucleotides, 10-15 nucleotides, or 12-15 nucleotides.

As used herein, the terms “upstream” and “downstream” refer to relative positions within a single nucleic acid (e.g., DNA) sequence. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the terms “upstream” and downstream” are used in reference to a non-PAM strand. For example, in some embodiments, a PBS is complementary to a non-PAM strand sequence that is upstream of a target sequence. As such, in some embodiments, a PBS binds to a sequence upstream of a sequence to which a spacer sequence binds, and the spacer sequence binds downstream of a sequence to which the PBS binds.

As used herein, the term “complex” refers to a grouping of two or more molecules. In some embodiments, the complex comprises a polypeptide and a nucleic acid molecule interacting with (e.g., binding to, coming into contact with, adhering to) one another. In some embodiments, the term “complex” is used to refer to association of a CRISPR nuclease (e.g., a Type V nuclease such as a Cas12i polypeptide) and a reverse transcriptase polypeptide. For example, a complex of a CRISPR nuclease (e.g., a Cas12i2 polypeptide as disclosed herein) and a reverse transcriptase polypeptide may be a heterodimer of the two polypeptides, e.g., via a dimerization domain (e.g., a leucine zipper), an antibody, a nanobody, or an aptamer. In some embodiments, the term “complex” is used to refer to association of an RNA guide and an RT donor RNA. In some embodiments, the term “complex” is used to refer to association of a CRISPR nuclease (e.g., a Type V nuclease such as a Cas12i polypeptide), a reverse transcriptase polypeptide, an RNA guide, and an RT donor RNA. In some embodiments, the term “complex” is used to refer to association of a reverse transcriptase polypeptide and an RT donor RNA.

As used herein, the terms “fusion” and “fused” refer to the joining of at least two nucleotide or protein molecules. For example, “fusion” and “fused” can refer to the joining of at least two polypeptide domains that are encoded by separate genes (e.g., a Type V nuclease and a reverse transcriptase polypeptide) in nature. The fusion can be an N-terminal fusion, a C-terminal fusion, or an intramolecular fusion. In some aspects, the domains are transcribed and translated to produce a single polypeptide. Also as used herein, the terms “fusion” and “fused” are used to refer to the joining of two nucleic acid molecules, such as two RNA molecules (e.g., an RNA guide and an RT donor RNA). The fusion can be a 5′ fusion, a 3′ fusion, or an intramolecular fusion.

As used herein, the term “edit” refers to one or more modifications introduced into a nucleotide sequence in a target nucleic acid such as in a genomic site of interest. The edit may occur within a target sequence as defined herein. Alternatively, the edit may occur outside the target sequence (e.g., adjacent to the target sequence). The edit can be one or more substitutions, one or more insertions, one or more deletions, or a combination thereof.

As used herein, the term “deletion” refers to a loss of a nucleotide or nucleotides in a nucleic acid sequence, relative to a reference sequence. No particular process is implied in how to make a sequence comprising a deletion. For instance, a sequence comprising a deletion can be synthesized directly from individual nucleotides. In other embodiments, a deletion is made by providing and then altering a reference sequence. The nucleic acid sequence can be in a genome of an organism. The nucleic acid sequence can be in a cell. The nucleic acid sequence can be a DNA sequence. The deletion can be a frameshift mutation or a non-frameshift mutation. A deletion described herein refers to an insertion of up to several kilobases.

As used herein, the term “insertion” refers to a gain of a nucleotide or nucleotides in a nucleic acid sequence, relative to a reference sequence. No particular process is implied in how to make a sequence comprising an insertion. For instance, a sequence comprising an insertion can be synthesized directly from individual nucleotides. In other embodiments, an insertion is made by providing and then altering a reference sequence. The nucleic acid sequence can be in a genome of an organism. The nucleic acid sequence can be in a cell. The nucleic acid sequence can be a DNA sequence. The insertion can be a frameshift mutation or a non-frameshift mutation. An insertion described herein refers to an insertion of up to several kilobases.

As used herein, the term “substitution” refers to a replacement of a nucleotide or nucleotides with a different nucleotide or nucleotides, relative to a reference sequence. No particular process is implied in how to make a sequence comprising a substitution. For instance, a sequence comprising a substitution can be synthesized directly from individual nucleotides. In other embodiments, a substitution is made by providing and then altering a reference sequence. The nucleic acid sequence can be in a genome of an organism. The nucleic acid sequence can be in a cell. The nucleic acid sequence can be a DNA sequence. The substitution described herein refers to a substitution of up to several kilobases.

### I. Gene Editing Systems

In some aspects, the present disclosure provides gene editing systems with enhanced gene editing efficiencies. The gene editing systems disclosed herein comprise one or more RNA components or nucleic acids encoding such, the one or more RNA components comprising a gRNA and optionally a reverse transcription RNA donor RNA. In some instances, the gRNA may comprise a spacer sequence and an engineered scaffold sequence recognizable by a Type V CRISPR nuclease (e.g., SEQ ID NO:1 or a variant thereof as disclosed herein). Such a gene editing system is more efficient in editing a genomic site targeted by the spacer sequence as compared with a gene editing system comprising a gRNA having the parent scaffold sequence and the same spacer sequence. The gene editing system disclosed herein further comprises one or more protein components, including a Type V CRISPR nuclease (e.g., SEQ ID NO:1 or a variant thereof as disclosed herein) and optionally a reverse transcriptase (RT). In some instances, the gene editing system comprise a fusion polypeptide comprising a variant of the Type V CRISPR nuclease and the RT. Such a gene editing system is more efficient in gene editing relative to a gene editing system comprising the wild-type counterpart of the Type V CRISPR nuclease.

### A. Gene Editing RNA Molecules

The gene editing systems disclosed herein comprise one or more gene editing RNA molecules, including a gRNA and optionally a reverse transcription donor RNA. In some instances, the gRNA and the reverse transcription donor RNA may form a single polynucleotide (e.g., an editing RNA template as disclosed herein). The gRNA may be an engineered gRNA, which comprises an engineered scaffold sequence relative to the parent scaffold sequence (e.g., SEQ ID NO: 71), which is specific to a Type V CRISPR nuclease (e.g., SEQ ID NO: 1).

(i) Engineered Guide RNAs (gRNAs)

The engineered gRNAs disclosed herein comprise a spacer sequence targeting a genomic site of interest and an engineered scaffold sequence recognizable by the Type V CRISPR nuclease of SEQ ID NO:1 or a variant thereof (e.g., those disclosed herein) to enhance editing efficiencies of the CRISPR nuclease.

### Engineered Scaffold Sequence

The gRNAs of the gene editing systems disclosed herein may comprise an engineered scaffold sequence relative to SEQ ID NO: 71 (the parent scaffold sequence for the Type V CRISPR nuclease of SEQ ID NO:1). The engineered scaffold sequence comprises one or more mutations relative to SEQ ID NO: 71 to improve gene editing efficiency of the Type V CRISPR nuclease. In some instances, the engineered scaffold sequence maybe about 115-135 nucleotides in length.

In some examples, the engineered scaffold sequence comprises nucleotide substitutions at one or more positions within SEQ ID NO: 71, for example, at one or more of positions 25 (e.g., A→C), 26 (e.g., U→C), 30 (e.g., A→C), 38 (e.g., A→C), 48 (e.g., U→G), 52 (e.g., U→G), 55 (e.g., U→G), 66 (e.g., A→G), 67 (e.g., U→C), 79 (e.g., A→G), 82-87 (e.g., A→U, C→U, C→G, A→G, A→G, and A→U or G, respectively), 91-93 (e.g., A→G, A→G, and U→G, respectively), 96 (e.g., U→C), 98-100 (e.g., A→G, U→C, and A→C, respectively), 104 (e.g., A→G), 105 (e.g., U→C), 107 (e.g., A→U), 110 (e.g., A→C), 113 (e.g., U→C), 115 (e.g., U→C), 118-121 (e.g., U→A, U→G, A→G, and U→C, respectively), and 123 (e.g., A→G) of SEQ ID NO: 71. In some examples, the engineered scaffold sequence may comprise nucleotide substitutions at positions 66-67 (e.g., AU→CC), 87 (e.g., A→U), 92-93 (e.g., AU→GG), 96 (e.g., U→C), 98 (e.g., A→G), 100 (e.g., A→C), 119 (e.g., U→G), 121 (e.g., U→C), and 123 (e.g., A→G) of SEQ ID NO: 71. In other examples, the engineered scaffold sequence may comprise nucleotide substitutions at positions 87 (e.g., A→U), 96 (e.g., U→C), 100 (e.g., A→C), 119 (e.g., U→G) and 123 (e.g., A→G) of SEQ ID NO: 71. In yet other examples, the engineered scaffold sequence may comprise nucleotide substitutions at positions 38 (e.g., A→C), 48 (e.g., U→G), 66 (e.g., A→G), 79 (e.g., A→G), 83 (e.g., C→U), 85 (e.g., A→G), 96 (e.g., G→U), 107 (e.g., A→U), 110 (e.g., A→C), 113 (e.g., U→C), 118-120 (e.g., UUA→AUG), and 123 (e.g., A→C) of SEQ ID NO: 71.

Alternatively or in addition, the engineered scaffold sequence may comprise one or more deletions at positions of 25-31, 52-55, and 84-87 of SEQ ID NO: 71. For example, the engineered scaffold sequence may have a deletion at position 25, 30, 31, 52, 55, 84, 86, or 87 of SEQ ID NO: 71. In other examples, the engineered scaffold sequence may have a deletion of positions 27 and 28, positions 85 and 86, or positions 86 and 87 of SEQ ID NO: 71. In yet other examples, the engineered scaffold sequence may have a deletion of positions 25-29, positions 26-30, positions 52-54, or positions 53-55 of SEQ ID NO: 71. In some specific examples, the engineered scaffold sequence may have deletions at positions 32 and 52 of SEQ ID NO: 71. In other specific examples, the engineered scaffold sequence may have deletions at positions 26-30, 52-54, and 85-86 of SEQ ID NO: 71. In yet other examples, the engineered scaffold sequence may have deletions at positions 26-30, 52-54, and 87 of SEQ ID NO: 71.

Alternatively or in addition, the engineered scaffold sequence may comprise one or more mutations (e.g., nucleotide substitution, deletion, insertion, or a combination thereof) within the region of 72-77 of SEQ ID NO: 71. Exemplary mutations include nucleotide substitution at position 72 (e.g., A→G), position 75 (e.g., U→A or C), position 76 (e.g., U→C), position 77 (e.g., U→C), or a combination thereof, deletion of position 77, deletion of positions 72-74 or 73-77, and/or insertions between positions 71 and 72 (e.g., insertion of GG), between positions 72 and 73 (e.g., insertion of GAU), between positions 75 and 76 (e.g., insertion of CU), and between positions 77 and 78 (e.g., insertion of CG). In some specific examples, the engineered scaffold sequence may have a nucleotide substitution at position 75 (e.g., U→A). In other specific examples, the engineered scaffold sequence may have a nucleotide substitution at position 75 (e.g., U→A) and deletions of positions 72-74 and 76-77 of SEQ ID NO: 71. In yet other specific examples, the engineered scaffold sequence may have a nucleotide substitution at position 77 (e.g., U→C) and insertions between positions 71 and 72 (e.g., insertion of GG) and between positions 75 and 76 (e.g., insertion of CU) of SEQ ID NO: 71.

In some instances, the engineered scaffold sequence may further comprise an insertion at 5′ end, between positions 107 and 108, and/or between positions 114 and 115, in combination of any of the nucleotide substitutions, deletions and/or mutations with positions 72-77 disclosed herein. For example, the engineered scaffold sequence may comprise one or more extra nucleotides at the 5′ end (e.g., insertion of C or UUC at the 5′ end). Alternatively, the engineered scaffold sequence may comprise an insertion at the 5′ end (e.g., insertion of C), an insertion between positions 107 ad 108 of SEQ ID NO: 71 (e.g., insertion of U), and an insertion between positions 114 and 115 of SEQ ID NO: 71 (e.g., insertion of GA).

Any of the engineered scaffold sequence as disclosed herein may have a nucleotide sequence at least 75% identical (e.g., at least 80%, at least 85%, at least 90%, at least 95% or higher) to SEQ ID NO: 71. An engineered gRNA comprising the engineered scaffold sequence disclosed herein show higher gene editing efficiency in a gene editing system comprising the Type V CRISPR nuclease or a variant thereof as disclosed herein as compared with gRNAs comprising the parent scaffold sequence of SEQ ID NO: 71.

Exemplary engineered scaffold sequences are provided in Tables 2 and 4, all of which are within the scope of the present disclosure.

### Spacer Sequences

The gRNAs disclosed herein (e.g., the engineered gRNA) comprises a spacer sequence for targeting a genomic site of interest. In some embodiments, the spacer sequence may be located at the 3′ end of the scaffold sequence. The spacer in any of the gRNA disclosed herein can be specific to a target sequence, i.e., capable of binding to the complementary region of the target sequence via base-pairing. In some instances, the target sequence may be within a genomic site of interest, e.g., where gene editing is needed. In some embodiments, the target sequence is adjacent to a PAM sequence, e.g., a PAM sequence for the Type V CRISPR nuclease as disclosed herein, for example, the motif of 5′-NTTN-3′ (or 5′-TTN-3′) wherein N is any nucleotide (e.g., A, G, T, or C). The PAM sequence is upstream to the target sequence. The PAM sequence may be immediately adjacent to the target sequence or, for example, within a small number (e.g., 1, 2, 3, 4, or 5) of nucleotides of the target sequence.

A spacer sequence as disclosed herein may have a length of from about 15 nucleotides to about 30 nucleotides. For example, the spacer can have a length of from about 15 nucleotides to about 20 nucleotides, from about 15 nucleotides to about 25 nucleotides, from about 20 nucleotides to about 25 nucleotides, or from about 20 nucleotides to about 30 nucleotides.

In some embodiments, the spacer in the gRNA may be generally designed to have a length of between 15 and 25 nucleotides (e.g., 15, 16, 17, 18, 19, 20, 21, 22, 23, 24, and 25) and be complementary to a specific target sequence. In some embodiments, the spacer sequence may be designed to have a length of between 18-22 nucleotides (e.g., 20 nucleotides).

In some embodiments, the spacer sequence may have at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a target sequence as described herein and is capable of binding to the complementary region of the target sequence via base-pairing.

In some embodiments, the spacer sequence comprises only RNA bases. In some embodiments, the spacer sequence comprises a DNA base (e.g., the spacer comprises at least one thymine). In some embodiments, the spacer sequence comprises RNA bases and DNA bases (e.g., the DNA-binding sequence comprises at least one thymine and at least one uracil).

In some instances, the gRNA disclosed herein may further comprise a linker sequence, a 5′ end and/or 3′ end protection fragment (see disclosures herein), or a combination thereof.

### (ii) Reverse Transcription Donor RNAs

In some embodiments, the gene editing system disclosed herein may further comprise a reverse transcription donor RNA (also known as reverse transcription template or RTT, specifically when the gene editing system also comprises an RT.

The RT donor RNA or RTT may comprise: (i) a primer binding site (PBS), and (ii) a reverse transcription template sequence. In some instances, the RT donor RNA may contain about 40-100 nucleotides, for example, about 40-80, about 40-60, or about 40-50 nucleotides. In some examples, the RT donor RNA may be about 45-nucleotide in length.

### Primer Binding Sites (PBS)

In some embodiments, the PBS in an RT donor RNA as disclosed herein is an RNA sequence capable of binding to a DNA strand via base-pairing. The DNA strand has been or can be nicked or cleaved by a CRISPR nuclease. In some embodiments, the PBS comprises an RNA sequence capable of binding to a DNA strand (a PBS-targeting site) via base-pairing. The DNA strand may have a free 3′ free end or a 3′ free end can be generated via cleavage by a CRISPR nuclease contained in the same gene editing system. In some examples, the PBS-targeting site may be located on the same DNA strand as the PAM sequence (the PAM strand). In some examples, the PBS-targeting site may be located on the complementary strand of the PAM strand (the non-PAM strand).

In some embodiments, the PBS is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length. In some embodiments, the PBS is about 3 nucleotides to about 200 nucleotides in length (e.g., about 3 nucleotides, 5 nucleotides, 8 nucleotides, 10 nucleotides, 13 nucleotides, 15 nucleotides, 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides or any length in between). In some embodiments, the PBS is about 3 nucleotides to about 100 nucleotides in length (e.g., about 3 nucleotides, 5 nucleotides, 8 nucleotides, 10 nucleotides, 13 nucleotides, 15 nucleotides, 20 nucleotides, 30 nucleotides, 40 nucleotides, 50 nucleotides, 60 nucleotides, 70 nucleotides, 80 nucleotides, 90 nucleotides, or 100 nucleotides or any length in between).

In some embodiments, the PBS is about 10 nucleotides to about 60 nucleotides in length. In some embodiments, the PBS is about 20 nucleotides to about 40 nucleotides in length. In some embodiments, the PBS is about 30 nucleotides in length.

In the gene editing system comprising the Type V nuclease as disclosed herein, the PBS in the RT donor RNA may bind to a region (the PBS-targeting site) on the non-PAM strand. In some embodiments, the PBS binds a PBS-targeting site that is adjacent to the complementary region of the target sequence. In some instances, the PBS-targeting site may be located upstream to the complementary region of the target sequence. For example, the PBS-targeting site may be up to 20 nucleotides upstream to the complementary region, for example, up to 15 nucleotides, up to 10 nucleotides, or up to 5 nucleotides. In specific examples, the PBS-targeting site may be about 3 nucleotides to about 10 nucleotides upstream of the complementary region. In specific examples, the PBS-targeting site may be 1 nucleotide, 1-2 nucleotides, 1-3 nucleotides, 1-4 nucleotides, 1-5 nucleotides, 1-6 nucleotides, 1-7 nucleotides, 1-8 nucleotides, 1-9 nucleotides, 1-10 nucleotides, 2-3 nucleotides, 2-4 nucleotides, 2-5 nucleotides, 2-6 nucleotides, 2-7 nucleotides, 2-8 nucleotides, 2-9 nucleotides, 2-10 nucleotides, 3-4 nucleotides, 3-5 nucleotides, 3-6 nucleotides, 3-7 nucleotides, 3-8 nucleotides, 3-9 nucleotides, 3-10 nucleotides, 4-5 nucleotides, 4-6 nucleotides, 4-7 nucleotides, 4-8 nucleotides, 4-9 nucleotides, 4-10 nucleotides, 5-6 nucleotides, 5-7 nucleotides, 5-8 nucleotides, 5-9 nucleotides, 5-10 nucleotides, 6-7 nucleotides, 6-8 nucleotides, 6-9 nucleotides, 6-10 nucleotides, 7-8 nucleotides, 7-9 nucleotides, 7-10 nucleotides, 8-9 nucleotides, 8-10 nucleotides, 9-10 nucleotides, or 10 nucleotides upstream of the complementary region. In other instances, the PBS-targeting site may overlap with the complementary region. When a free 3′ end is generated by the Type V CRISPR nuclease in the gene editing system within or nearby the target sequence and the complementary region, the PBS binding to the non-PAM strand at a site upstream to or overlapping with the complementary region could efficiently facilitate DNA synthesis by the RT polypeptide in the gene editing system, starting from the free 3′ end generated in the non-PAM strand.

### Reverse Transcription Template (RTT) Sequences

The reverse transcription template sequence (template sequence) serves as the template for the reverse transcription mediated by the RT polypeptide in the gene editing system disclosed herein. In some embodiments, the reverse transcription template sequence is homologous to the genomic site of interest and may comprise one or more nucleotide variations relative to the genomic site of interest. In some embodiments, the reverse transcription template sequence is about 5-100-nucleotide in length. In some embodiments, the reverse transcription template sequence is about 10-90-nucleotide in length. In some embodiments, the reverse transcription template sequence is about 20-80-nucleotide in length. In some embodiments, the reverse transcription template sequence is about 30-70-nucleotide in length. In some embodiments, the reverse transcription template sequence is about 40-60-nucleotide in length. In some embodiments, the reverse transcription template sequence is about 30-90-nucleotide in length. In some instances, the reverse transcription template sequence is about 45 nucleotides.

The reverse transcription template sequence can be transcribed into DNA by the reverse transcriptase of the gene editing system described herein. In some embodiments, the reverse transcription template sequence is transcribed from 5′ to 3′ into DNA of the PAM strand. In some embodiments, the reverse transcription template sequence is transcribed from 5′ to 3′ into DNA of the non-PAM strand. In some embodiments, the reverse transcription template sequence is transcribed from 5′ to 3′ into DNA of the PAM strand. In some embodiments, the reverse transcription template sequence is transcribed from 5′ to 3′ into DNA of the non-PAM strand. In some embodiments, the reverse transcription template sequence is 5′ of the PBS. In some embodiments, the reverse transcription template sequence is 3′ of the PBS. In some embodiments, the reverse transcription template sequence is transcribed into DNA of the PAM strand through 3′ extension from the PBS. In some embodiments, the reverse transcription template sequence is transcribed into DNA of the non-PAM strand through 3′ extension from the PBS.

(iii) Editing Template RNAs or Gene Editing RNAs

In some instances, the gene editing system disclosed herein comprise a polynucleotide (e.g., an editing template RNA or gene editing RNA) that comprises the gRNA and the RRT as disclosed herein. In some examples, the gRNA may be upstream to the RRT in the editing template RNA. Alternatively, the RRT may be upstream to the gRNA in the editing template RNA. In some examples, the gRNA and the RRT are connected via an oligonucleotide linker, for example, a polyA linker, which may comprise about 3-10 A residues. In specific examples, the gRNA and the RRT can be connected via a A5 linker.

In some embodiments, the editing template RNA as disclosed herein may comprise one or more additional elements, for example, a U6 start motif, end protection fragments, U6 termination signal motif, or a combination thereof.

In some examples, the editing template RNA (or the gRNA and/or the RT donor RNA as disclosed herein), may comprise one or more protection fragments at either or both ends of the RNA molecules. Alternatively or in addition, the editing template RNA, or the gRNA and/or the RT donor RNA thereof, may comprise additional elements internal to the RNA molecule (e.g., between one or more of the sequences in the editing template RNA, e.g., between a PBS and a reverse transcription template sequence, e.g., a linker). In some embodiments, the editing template RNA comprises additional elements between one or more sequence of the editing template RNA, e.g., such as an RNA guide (a nuclease binding sequence or a DNA-binding sequence) or an RT donor RNA (a PBS or a reverse transcription template sequence).

In some embodiments, the editing template RNA comprises additional elements, e.g., a direct repeat sequence, at one or more ends. In some embodiments, the direct repeat sequence may recruit a Type V CRISPR nuclease (e.g., a variant Type V nuclease or a variant Type V nuclease-reverse transcriptase fusion polypeptide).

In some embodiment, the additional elements may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In some examples, the editing template RNA may comprise an optional nucleotide linker. Such an optional nucleotide linker sequence may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length. In some embodiments, the optional nucleotide linker is between any of the nuclease binding sequence, the DNA-binding sequence, the PBS and/or reverse transcription template sequence.

In some examples, the 5′ end and/or the 3′ end of the editing template RNA, or the gRNA and/or the RT donor RNA thereof, may contain a protection fragment, which may enhance resistance of the RNA molecule to exonuclease activity. In some instances, the end protection fragment may comprise a nucleotide sequence capable of forming a secondary structure, such as hairpin, a pseudoknot, or a triplex structure. In other instances, the end protection fragment may comprise the sequence of an exoribonuclease-resistant RNA (xrRNA), a transfer RNA (tRNA), or a truncated tRNA. In some embodiments, the modification is a Zika-like pseudoknot, a murine leukemia virus pseudoknot (MLV-PK) sequence, a red clover necrotic mosaic virus (RCNMV) sequence, a sweet clover necrotic mosaic virus (SCNMV) sequence, a carnation ringspot virus (CRSV) sequence, preQ sequence, or an RNA bacteriophage MS2 sequence. In specific examples, the end protection fragment may comprise one or more CRISPR nuclease binding sites (e.g., bindings sites for the Type V CRISPR nuclease as disclosed herein), and optionally one or more segments (e.g., spacers) that share no homology with any human sequences. In some instances, the one or more segments bind to a sequence that is no more than 85% identical to any sequence of the human genome. Such an end protection fragment can recruit the CRISPR nuclease contained in the same gene editing system to inhibit exoribonuclease activity without inducing off-target gene edits.

### (iv) Modification of Nucleic Acids

Any of the RNA components in a gene editing system as disclosed herein, e.g., the editing template RNA, the gRNA, the RT donor RNA, may include one or more modifications.

Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The gRNA or any of the nucleic acid sequences encoding components of the composition may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 10% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotides (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, any RNA sequence described herein, such as an editing template RNA, may comprise an end modification (e.g., a 5′ end modification or a 3′ end modification). In some embodiments, the end modification is a chemical modification. In some embodiments, the end modification is a structural modification. See disclosures herein.

When a gene editing system disclosed herein comprises nucleic acids encoding the CRISPR nuclease and/or the RT polypeptide, e.g., mRNA molecules, such nucleic acid molecules may contain any of the modifications disclosed herein, where applicable.

### B. Gene Editing Protein Molecules

Any of the gene editing systems disclosed herein may comprises a Type V CRISPR nuclease (e.g., SEQ ID NO:1 or a variant thereof), and optionally a reverse transcriptase (RT). In some embodiments, the gene editing system may comprise a fusion polypeptide comprising the Type V CRISPR nuclease and the RT.

### (i) Type V CRISPR Nuclease

In some embodiments, the gene editing system disclosed herein comprises the Type V CRISPR nuclease set forth as SEQ ID NO: 1 or a variant thereof. In some instances, the variant of SEQ ID NO: 1 show enhanced gene editing activity as compared with the wild-type counterpart (SEQ ID NO: 1). As reported herein, various mutations were introduced into the CRISPR nuclease of SEQ ID NO:1 to identify those that would result in variants with enhanced enzymatic activities. Exemplary mutations and the gene editing efficiency of the resultant variants (either alone or in fusion with an RT) were provided in Table 8 below. Such variants, specifically those that show enhanced gene editing efficiencies, are within the scope of the present disclosure.

Any of the variant of SEQ ID NO:1 may comprise an amino acid sequence at least 80% (e.g., at least 85%, at least 90%, at least 95%, at least 98%, or higher) identical to SEQ ID NO: 1.

In some embodiments, the Type V CRISPR nuclease disclosed herein is a variant of SEQ ID NO:1 that comprises amino acid substitutions at position S136, D137, and/or A138. For example, such a variant may comprise amino acid substitutions of S136G, D137G, and/or A138R. Alternatively, the variant may comprise amino acid substitutions of S136G, D137R, and/or A138R.

In some instances, the Type V CRISPR nuclease may comprise an amino- and/or carboxyl-terminal extensions. For example, the CRISPR nuclease may contain additional peptides, e.g., an epitope peptide for labelling, such as a polyhistidine tag (His-tag), Myc, and FLAG. In some embodiments, the CRISPR nuclease described herein can be fused to a detectable moiety such as a fluorescent protein (e.g., green fluorescent protein (GFP) or yellow fluorescent protein (YFP)).

Alternatively or in addition, the CRISPR nuclease as in any one of the embodiments described herein interacts with a reverse transcriptase polypeptide (e.g., through electrostatic interactions). In some embodiments, the CRISPR nuclease comprises a dimerization domain. As used herein, the term “dimerization domain,” refers to a polypeptide domain capable of specifically binding a separate, and compatible, polypeptide domain (e.g., a second compatible dimerization domain). In some embodiments, the dimer is formed by a non-covalent bond between the first dimerization domain and the second compatible dimerization domain. In some embodiments, a dimerization domain is a leucine zipper, nanobody, or antibody. In some embodiments, the dimerization domain recruits a reverse transcriptase polypeptide. In some embodiments, the CRISPR nuclease and the reverse transcriptase polypeptide interact through coiled-coil peptide heterodimers.

In some embodiments, the CRISPR nuclease as in any one of the embodiments described herein interacts with a ligase, an integrase, and/or a recombinase. In some embodiments, the CRISPR nuclease as in any one of the embodiments described herein is fused to a ligase, an integrase, and/or a recombinase. In some embodiments, the ligase, integrase, and/or recombinase is fused to the N-terminus or C-terminus of the CRISPR nuclease. In some embodiments, the ligase, integrase, and/or recombinase is fused internally to the CRISPR nuclease. In some embodiments, the integrase is a serine integrase. In some embodiments, the integrase is a Bxb1, TP901, or PhiBT1 integrase. In some embodiments, the recombinase is a serine recombinase or a tyrosine recombinase. In some embodiments, the recombinase is a CRE recombinase. In some embodiments, a CRISPR nuclease that interacts with or is fused to a ligase, integrase, and/or recombinase further interacts with or is fused to a reverse transcriptase.

### (ii) Reverse Transcriptase

In some embodiments, the gene editing system disclosed herein may further comprise a polymerase (e.g., DNA-dependent DNA polymerase or RNA-dependent DNA polymerase), or a variant thereof, which can be provided as a fusion to the CRISPR nuclease. The polymerase may be a wild-type polymerase, functional fragment, variant, truncated variant, or the like. The polymerase may include a wild-type polymerase from eukaryotic, prokaryotic, archaeal, or viral organisms, and/or the polymerases may be modified by genetic engineering, mutagenesis, directed evolution-based processes.

In some embodiments, the polymerase is a reverse transcriptase. In some embodiments, the reverse transcriptase polypeptide is any wild-type reverse transcriptase obtained from any naturally-occurring organism or virus, or obtained from a commercial or non-commercial source. The reverse transcriptase polypeptide may also be a variant reverse transcriptase polypeptide.

The reverse transcriptase polypeptide can be obtained from a number of different sources. For instance, the gene may be obtained from eukaryotic cells which are infected with retrovirus or from a plasmid that comprises either a portion of or the entire retrovirus genome. In addition, RNA that comprises the reverse transcriptase gene can be obtained from retroviruses.

A person of ordinary skill in the art will recognize that reverse transcriptases are known in the art, including, but not limited to, Moloney Murine Leukemia Virus (MMLV) reverse transcriptase, Human Immunodeficiency Virus (HIV) reverse transcriptase, and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the composition described herein.

In some embodiments, the reverse transcriptase is MMLV-RT, MarathonRT from Eubacterium rectale, or RTX reverse transcriptase or a variant of MMLV-RT, MarathonRT, or RTX reverse transcriptase.

In some embodiments, the reverse transcriptase polypeptide is fused to a CRISPR nuclease (e.g., the Type V CRISPR nuclease of SEQ ID NO:1 or a variant thereof as disclosed herein) as in any one of the embodiments described herein. In some embodiments, the reverse transcriptase polypeptide comprises an N-terminal CRISPR nuclease. In some embodiments, the reverse transcriptase polypeptide comprises a C-terminal CRISPR nuclease. In some embodiments, the reverse transcriptase polypeptide comprises a CRISPR nuclease at an intramolecular position within the reverse transcriptase polypeptide (e.g., the CRISPR nuclease) is within a loop of the reverse transcriptase polypeptide.

Any of the CRISPR nuclease-RT fusion polypeptides, such as those disclosed herein (e.g., those shown in Table 1 and Table 8), their encoding nucleic acids, vectors comprising such and method of making such are also within the scope of the present disclosure.

In some embodiments, a CRISPR nuclease-reverse transcriptase fusion polypeptide as described elsewhere herein is capable of binding and binds to at least one nuclease binding sequence in the editing template RNA. In some embodiments, the CRISPR nuclease-reverse transcriptase fusion polypeptide is capable of binding and binds to a target sequence through at least one DNA-binding sequence in the editing template RNA. In such embodiments, the CRISPR nuclease-reverse transcriptase fusion polypeptide is recruited to or brought in close proximity to the target sequence through binding of the CRISPR nuclease via the nuclease binding sequence and the DNA-binding sequence of the editing template RNA.

In some embodiments, the reverse transcriptase transcribes the reverse transcription template sequence into the non-PAM strand of a target nucleic acid starting at the 5′ end of a PBS. In some embodiments, the reverse transcriptase transcribes the reverse transcription template sequence into the non-PAM strand of a target nucleic acid starting at the 3′ end of a PBS. In some embodiments, the reverse transcriptase transcribes the reverse transcription template sequence into the PAM strand of a target nucleic acid starting at the 5′ end of a PBS. In some embodiments, the reverse transcriptase transcribes the reverse transcription template sequence into the PAM strand of a target nucleic acid starting at the 3′ end of a PBS. In some embodiments, following binding of a PBS to a non-PAM strand of a target nucleic acid, the reverse transcriptase transcribes the reverse transcription template sequence from a free 3′ end of the non-PAM strand. In some embodiments, following hybridization of a PBS to a PAM strand of a target nucleic acid, the reverse transcriptase transcribes the reverse transcription template sequence from a free 3′ end of the PAM strand.

Alternatively, the reverse transcriptase polypeptide comprises a dimerization domain. In some embodiments, a dimerization domain is a leucine zipper, nanobody, or antibody. In some embodiments, the dimerization domain recruits a Type V CRISPR nuclease.

In some embodiments, the reverse transcriptase as in any one of the embodiments described herein interacts with a ligase, an integrase, and/or a recombinase. In some embodiments, the reverse transcriptase as in any one of the embodiments described herein is fused to a ligase, an integrase, and/or a recombinase. In some embodiments, the ligase, integrase, and/or recombinase is fused to the N-terminus or C-terminus of the reverse transcriptase. In some embodiments, the ligase, integrase, and/or recombinase is fused internally to the reverse transcriptase. In some embodiments, the integrase is a serine integrase. In some embodiments, the integrase is a Bxb1, TP901, or PhiBT1 integrase. In some embodiments, the recombinase is a serine recombinase or a tyrosine recombinase. In some embodiments, the recombinase is a CRE recombinase. In some embodiments, a reverse transcriptase that interacts with or is fused to a ligase, integrase, and/or recombinase further interacts with or is fused to a CRISPR nuclease.

### C. Exemplary Gene Editing Systems

In some embodiments, a gene editing system as disclosed herein may comprise the protein components of the Type V CRISPR nuclease, the RT polypeptide, or both. Alternatively, the gene editing system may comprise one or more nucleic acids (e.g., vectors such as viral vectors) encoding the protein components. In some examples, the gene editing system may comprise one vector encoding both the Type V CRISPR nuclease and the RT polypeptide. Alternatively or in addition, a gene editing system as disclosed herein may comprise the RNA components of the gene editing RNA, the engineered gRNA, or both. Alternatively, the gene editing system may comprise one or more nucleic acids (vectors) encoding the RNA components. For example, the gene editing system may comprise one vector (e.g., a viral vector such as an AAV vector, e.g., AAV1, AAV2, AAV3, AAV4, AAV5, AAV6, AAV7, AAV8, AAV9, AAVrh10, AAV11 and AAV12) coding for both the gene editing RNA and the gRNA.

In some examples, a gene editing system as disclosed herein may comprise the protein components of the Type V CRISPR nuclease, the RT polypeptide, or both, and the RNA components of gene editing RNA and the engineered gRNA. In other examples, a gene editing system as disclosed herein may comprise the protein components of the Type V CRISPR nuclease, the RT polypeptide, or both, and one or more nucleic acids encoding the RNA components of gene editing RNA and the engineered gRNA. In yet other examples, a gene editing system as disclosed herein may comprise one or more nucleic acids encoding the protein components of the Type V CRISPR nuclease, the RT polypeptide, or both, and the RNA components of gene editing RNA and the engineered gRNA. Alternatively, a gene editing system as disclosed herein may comprise one or more nucleic acids encoding the protein components of the Type V CRISPR nuclease, the RT polypeptide, or both, and one of more nucleic acids encoding the RNA components of gene editing RNA and the engineered gRNA. In some instances, the gene editing system may comprise one vector encoding multiple components of the gene editing system. In some instances, the nucleic acid(s) encoding the Type V CRISPR nuclease, the RT polypeptide, and/or a fusion polypeptide thereof can be one or more mRNA molecules. In some examples, the mRNA molecule(s) may be codon optimized.

The exemplary gene editing systems described herein are meant to be illustrative only.

In some embodiments, a gene editing system as disclosed herein comprises (a) an engineered guide RNA (gRNA) (or engineered RNA guide, which are used herein interchangeably) as disclosed herein or a nucleic acid encoding such and (b) a Type V CRISPR nuclease (e.g., SEQ ID NO:1 or a variant thereof ad disclosed herein), or a nucleic acid encoding such. In some instances, the gene editing system comprises a nucleic acid encoding the Type V CRISPR nuclease. In some examples, the nucleic acid is a DNA molecule (e.g., a vector such as a viral vector). Alternatively, the nucleic acid is a messenger RNA (mRNA).

In some embodiments, a gene editing system as disclosed herein comprises (a) an engineered guide RNA (gRNA) (or engineered RNA guide, which are used herein interchangeably) as disclosed herein or a nucleic acid encoding such, (b) an RRT RNA or a nucleic acid encoding such, (c) a Type V CRISPR nuclease (e.g., SEQ ID NO:1 or a variant thereof ad disclosed herein), or a nucleic acid encoding such, and (d) a reverse transcriptase or a nucleic acid encoding such. In some instances, the gene editing system comprises one or more nucleic acids encoding the Type V CRISPR nuclease and/or the RT. In some examples, the nucleic acid(s) is a DNA molecule (e.g., a vector such as a viral vector). Alternatively, the nucleic acid(s) is a messenger RNA (mRNA).

In some embodiments, a gene editing system as disclosed herein comprises (a) an engineered guide RNA (gRNA) (or engineered RNA guide, which are used herein interchangeably) as disclosed herein or a nucleic acid encoding such, (b) an RRT RNA or a nucleic acid encoding such, and (c) a fusion polypeptide comprising a Type V CRISPR nuclease (e.g., SEQ ID NO:1 or a variant thereof ad disclosed herein) and an RT, or a nucleic acid encoding the fusion polypeptide. In some instances, the gene editing system comprises a nucleic acid encoding the fusion polypeptide. In some examples, the nucleic acid is a DNA molecule (e.g., a vector such as a viral vector). Alternatively, the nucleic acid is a messenger RNA (mRNA).

In some embodiments, a gene editing system as disclosed herein comprises (a) a polynucleotide (an editing RNA template) comprising an engineered guide RNA (gRNA) as disclosed herein and an RRT RNA, optionally any of the additional elements as also disclosed herein, or a nucleic acid encoding such, (b) a Type V CRISPR nuclease (e.g., SEQ ID NO:1 or a variant thereof ad disclosed herein), or a nucleic acid encoding such, and (c) a reverse transcriptase or a nucleic acid encoding such. In some instances, the gene editing system comprises one or more nucleic acids encoding the Type V CRISPR nuclease and/or the RT. In some examples, the nucleic acid(s) is a DNA molecule (e.g., a vector such as a viral vector). Alternatively, the nucleic acid(s) is a messenger RNA (mRNA).

In some embodiments, a gene editing system as disclosed herein comprises (a) a polynucleotide (an editing RNA template) comprising an engineered guide RNA (gRNA) as disclosed herein and an RRT RNA, optionally any of the additional elements as also disclosed herein, or a nucleic acid encoding such, and (b) a fusion polypeptide comprising a Type V CRISPR nuclease (e.g., SEQ ID NO:1 or a variant thereof ad disclosed herein), and a reverse transcriptase or a nucleic acid encoding the fusion polypeptide. In some instances, the gene editing system comprises a nucleic acid encoding the fusion polypeptide. In some examples, the nucleic acid is a DNA molecule (e.g., a vector such as a viral vector). Alternatively, the nucleic acid is a messenger RNA (mRNA).

In some embodiments, the gene editing system disclosed herein comprises one or more lipid nanoparticles (LNPs) in association with (e.g., encompassing) one or more of the protein and/or RNA components of the gene editing system, or their encoding nucleic acids. In other embodiments, the gene editing system may comprise one or more LNPs encompass a portion the components and one or more vectors encoding the remaining components.

### II. Preparation of Gene Editing System Components

The protein components, the RNA components, or their encoding nucleic acids (e.g., vectors or mRNAs) may be prepared by conventional methods of the methods disclosed herein.

In some embodiments, a Type V nuclease as disclosed herein, a reverse transcriptase, or a Type V CRISPR nuclease-reverse transcriptase fusion can be prepared by (a) culturing host cells such as bacteria cells or mammalian cells, capable of producing the proteins, isolating the proteins thus produced, and optionally, purifying the proteins. The CRISPR nuclease, the reverse transcriptase, or the fusion protein thus prepared may be complexed with the editing template RNA.

The Type V CRISPR nuclease and the reverse transcriptase can be also prepared by (b) a known genetic engineering technique, specifically, by isolating a gene encoding the Type V CRISPR nuclease and the reverse transcriptase of the present invention from bacteria, constructing a recombinant expression vector, and then transferring the vector into an appropriate host cell that expresses the editing template RNA for expression of a recombinant protein that complexes with the editing template RNA in the host cell. Alternatively, the Type V CRISPR nuclease and the reverse transcriptase can be prepared by (c) an in vitro coupled transcription-translation system and then complexes with editing template RNA. Bacteria that can be used for preparation of the Type V CRISPR nuclease and the reverse transcriptase of the present invention are not particularly limited as long as they can produce the Type V CRISPR nuclease and the reverse transcriptase of the present invention. Some nonlimiting examples of the bacteria include E. coli cells described herein.

Unless otherwise noted, all compositions and complexes and polypeptides provided herein are made in reference to the active level of that composition or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition unless otherwise indicated. In the exemplified composition, the enzymatic levels are expressed by pure enzyme by weight of the total composition and unless otherwise specified, the ingredients are expressed by weight of the total compositions.

### A. Vectors

The present disclosure provides one or more vectors for expressing the CRISPR nuclease, the reverse transcriptase, or their fusion polypeptide described herein or nucleic acids encoding the components described herein may be incorporated into a vector. In some embodiments, a vector disclosed herein includes a nucleotide sequence encoding CRISPR nuclease, the reverse transcriptase, or the fusion polypeptide. The present disclosure also provides one or more vectors encoding the editing template RNA or any portion thereof, e.g., the gRNA, or the RT donor RNA. In some embodiments, the vector comprises a Pol II promoter or a Pol III promoter.

Expression of natural or synthetic polynucleotides is typically achieved by operably linking a polynucleotide encoding the gene of interest, e.g., nucleotide sequence encoding the CRISPR nuclease, the reverse transcriptase, or the fusion polypeptide, and/or the editing template RNA, to a promoter and incorporating the construct into an expression vector. The expression vector is not particularly limited as long as it includes a polynucleotide encoding the CRISPR nuclease and the reverse transcriptase and/or the editing template RNA of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the polypeptide(s) from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the polypeptide(s) of the present invention has been transferred into the host cells and then expressed without fail.

The preparation method for recombinant expression vectors is not particularly limited, and examples thereof include methods using a plasmid, a phage or a cosmid.

### B. Methods of Expression

The present disclosure includes a method for protein expression, comprising translating the CRISPR nuclease and the reverse transcriptase, and expressing the editing template RNA described herein.

In some embodiments, a host cell described herein is used to express the CRISPR nuclease and the reverse transcriptase and/or the editing template RNA. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Type V CRISPR nuclease, the reverse transcriptase and/or the editing template RNA. After expression of the CRISPR nuclease, the reverse transcriptase and/or the editing template RNA, the host cells can be collected and CRISPR nuclease, the reverse transcriptase and/or the editing template RNA purified from the cultures etc. according to conventional methods (for example, filtration, centrifugation, cell disruption, gel filtration chromatography, ion exchange chromatography, etc.).

In some embodiments, the methods for CRISPR nuclease and the reverse transcriptase expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the polypeptide(s). In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the CRISPR nuclease and the reverse transcriptase.

A variety of methods can be used to determine the level of production of a mature CRISPR nuclease, the reverse transcriptase and/or the editing template RNA in a host cell. Such methods include, but are not limited to, for example, methods that utilize either polyclonal or monoclonal antibodies specific for the proteins or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the CRISPR nuclease and the reverse transcriptase and/or the editing template RNA in a cell, comprising providing a polyribonucleotide encoding the CRISPR nuclease, the reverse transcriptase and/or the editing template RNA to a host cell wherein the polyribonucleotide encodes the CRISPR nuclease, the reverse transcriptase and/or the editing template RNA, expressing the CRISPR nuclease, the reverse transcriptase and/or the editing template RNA in the cell, and obtaining the CRISPR nuclease, the reverse transcriptase and/or the editing template RNA from the cell.

### III. Methods for Gene Editing

Any of the gene editing systems can be used to genetically modify (edit) a target nucleic acid, which can be a genetic site of interest, e.g., a genetic site where genetic editing is needed, for example, to fix a genetic mutation, to introduce a protective mutation, to introduce modifications for modulating expression of a gene, etc.

The gene editing systems and compositions disclosed herein are applicable for editing and introducing edits into a variety of target sequences. In some embodiments, the target sequence is a DNA molecule, such as a DNA locus (referred to herein as a target sequence or an on-target sequence). In some embodiments, the target sequence is an RNA, such as an RNA locus or mRNA. In some embodiments, the target sequence is single-stranded (e.g., single-stranded DNA). In some embodiments, the target sequence is double-stranded (e.g., double-stranded DNA). In some embodiments, the target sequence comprises both single-stranded and double-stranded regions. In some embodiments, the target sequence is linear. In some embodiments, the target sequence is circular. In some embodiments, the target sequence comprises one or more modified nucleotides, such as methylated nucleotides, damaged nucleotides, or nucleotides analogs. In some embodiments, the target sequence is not modified. In some embodiments, a single-stranded target sequence does not require a PAM sequence.

The target sequence may be of any length, such as about at least any one of 100 bp, 200 bp, 500 bp, 1000 bp, 2000 bp, 5000 bp, 10 kb, 20 kb, 50 kb, 100 kb, 200 kb, 500 kb, 1 Mb, or longer. The target sequence may also comprise any sequence. In some embodiments, the target sequence is GC-rich, such as having at least about any one of 40%, 45%, 50%, 55%, 60%, 65%, or higher GC content. In some embodiments, the target sequence has a GC content of at least about 70%, 80%, or more. In some embodiments, the target sequence is a GC-rich fragment in a non-GC-rich target sequence. In some embodiments, the target sequence is not GC-rich. In some embodiments, the target sequence has one or more secondary structures or higher-order structures. In some embodiments, the target sequence is not in a condensed state, such as in a chromatin, to render the target sequence inaccessible by ribonucleoprotein.

In some embodiments, the target nucleic acid is a genomic site in a cell. In some instances, the target nucleic acid where the genetic edit would occur can be in a protein-coding region. Alternatively, the target nucleic acid may be in a regulatory region, such as a promoter, enhancer, a 5′ or 3′ untranslated region. In other instances, the target nucleic acid can be in a non-coding gene, such as transposon, miRNA, tRNA, ribosomal RNA, ribozyme, or lincRNA.

### A. Exemplary Genes for Genetic Editing

Any of the gene editing systems disclosed herein may be used to edit a target gene of interest, e.g., a gene involved in a disease (e.g., a genetic disease). In some embodiments, the target gene can be one that is involved in an immune response in a subject. For example, the target gene can be an immune checkpoint gene.

Exemplary target genes include, but are not limited to, BCL11A intronic erythroid enhancer, CD3, Beta-2 microglobulin (B2M), T Cell Receptor Alpha Constant (TRAC), Programmed Cell Death 1 (PDCD1), T-cell receptor alpha, T-cell receptor beta, B-cell lymphoma/leukemia 11A (BCL11A), Cytotoxic T-Lymphocyte Antigen 4 (CTLA-4), chemokine (C—C motif) receptor 5 (gene/pseudogene) (CCR5), CXCR4 gene, CD160 molecule (CD160), adenosine A2a receptor (ADORA), CD276, B7-H3, B7-H4, BTLA, nicotinamide adenine dinucleotide phosphate NADPH oxidase isoform 2 (NOX2), V-domain Ig suppressor of T cell activation (VISTA), Sialic acid-binding immunoglobulin-type lectin 7 (SIGLEC7), Sialic acid-binding immunoglobulin-type lectin 9 (SIGLEC9), SIGLEC10, V-set domain containing T cell activation inhibitor 1 (VTCN1), B and T lymphocyte associated (BTLA), Indoleamine 2,3-dioxygenase (IDO), indoleamine 2,3-dioxygenase 1 (IDO1), Killer-cell Immunoglobulin-like Receptor (KIR), killer cell immunoglobulin-like receptor, three domains, long cytoplasmic tail, 1 (KIR3DL1), lymphocyte-activation gene 3 (LAG3), T-cell Immunoglobulin domain and Mucin domain 3 (TIM3), hepatitis A virus cellular receptor 2 (HAVCR2), natural killer cell receptor 2B4 (CD244), hypoxanthine phosphoribosyltransferase 1 (HPRT), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), adeno-associated virus integration site 1 (AAVS1), AAVS 2, AAVS3, AAVS4, AAVS5, AAVS6, AAVS7, AAVS8, transforming growth factor beta receptor II (TGFBRII), transforming growth factor beta receptor 1 (TGFBR1), SMAD family member 2 (SMAD2), SMAD family member 3 (SMAD3), SMAD family member 4 (SMAD4), SKI proto-oncogene (SKI), SKI-like proto-oncogene (SKIL), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPPlR12C), TGFB induced factor homeobox 1 (TGIF1), tumor necrosis factor receptor superfamily member, tumor necrosis factor receptor superfamily member 10b (TNFRSF10B), tumor necrosis factor receptor superfamily member 10a (TNFRSF10A), BY55, B7H5, caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), guanylate cyclase 1, soluble, beta 3 (GUCYlB3), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCYlB2), prolyl hydroxylase domain (PHD1, PHD2, PHD3) family of proteins, CD27, CD28, CD40, CD122, CD137, OX40, GITR, and ICOS. In some embodiments, the modified gene is programmed death ligand 1 (PD-L1), class II major histocompatibility complex transactivator (CIITA), citramalyl-CoA lyase (CLYBL), transthyretin (TTR), lactate dehydrogenase-A (LDHA), dydroxyacid oxidase-1 (HAO1), alanine-glyoxylate and serine-pyruvate aminotransferase (AGXT), glyoxylate reductase/hydroxypyruvate reductase (GRHPR), 4-hydroxy-2-oxoglutarate aldolase (HOGA), polypyrimidine tract binding protein 1 (PTBP1), stathmin 2 (STMN2), or actin beta (ACTB).

The present disclosure provides methods for genetically editing any of the target genes as disclosed herein using the gene editing system as also disclosed herein.

### B. Edits

In some aspects, provided herein are methods for introducing at least one edit into a target nucleic acid (e.g., a genomic site of interest such as in any of the target genes disclosed herein) using the gene editing system described herein. In some embodiments, the edit may include a substitution, an insertion, a deletion, or a combination thereof, into the target nucleic acid. In some examples, the edit can be a single nucleotide substitution, such as a G to T substitution, a G to A substitution, a G to C substitution, a T to G substitution, a T to A substitution, a T to C substitution, a C to G substitution, a C to T substitution, a C to A substitution, an A to T substitution, an A to G substitution, or an A to C substitution. In some examples, the edit can convert a G:C base pair to a T:A base pair, a G:C base pair to an A:T base pair, a G:C base pair to C:G base pair, a T:A base pair to a G:C base pair, a T:A base pair to an A:T base pair, a T:A base pair to a C:G base pair, a C:G base pair to a G:C base pair, a C:G base pair to a T:A base pair, a C:G base pair to an A:T base pair, an A:T base pair to a T:A base pair, an A:T base pair to a G:C base pair, or an A:T base pair to a C:G base pair.

In some embodiments, a method is described for introducing at least one edit into a target nucleic acid, where the edit is at least one substitution, at least one insertion, and/or at least one deletion. In some embodiments, the edit comprises at least one substitution, insertion, or deletion. In some embodiments, the substitution, insertion, or deletion is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides in length. In some embodiments, the substitution, insertion, or deletion is from 1 nucleotide to about 200 nucleotides in length, e.g., 1 nucleotide to 5 nucleotides, from 5 nucleotides to 10 nucleotides, from 10 nucleotides to 15 nucleotides, from 15 nucleotides to 20 nucleotides, from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, or from 195 nucleotides to 200 nucleotides. In some embodiments, the substitution, insertion, or deletion is from 1 nucleotide to about 300 nucleotides in length, e.g., 1 nucleotide to 5 nucleotides, from 5 nucleotides to 10 nucleotides, from 10 nucleotides to 15 nucleotides, from 15 nucleotides to 20 nucleotides, from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, from 195 nucleotides to 200 nucleotides, from 200 nucleotides to 210 nucleotides, from 210 nucleotides to 220 nucleotides, from 220 nucleotides to 230 nucleotides, from 230 nucleotides to 240 nucleotides, from 240 nucleotides to 250 nucleotides, from 250 nucleotides to 260 nucleotides, from 260 nucleotides to 270 nucleotides, from 270 nucleotides to 280 nucleotides, from 280 nucleotides to 290 nucleotides, or from 290 nucleotides to 300 nucleotides. In some embodiments, the substitution, insertion, or deletion is up to about 10,000 base pairs (10 kb) in length. For example, in some embodiments, the substitution, insertion, or deletion is 1 base pair, about 10 base pairs, about 20 base pairs, about 30 base pairs, about 40 base pairs, about 50 base pairs, about 60 base pairs, about 70 base pairs, about 80 base pairs, about 90 base pairs, about 100 base pairs, about 200 base pairs, about 300 base pairs, about 400 base pairs, about 500 base pairs, about 600 base pairs, about 700 base pairs, about 800 base pairs, about 900 base pairs, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, 3 kb, 4 kb, 5 kb, 6 kb, 7 kb, 8 kb, 9 kb, or 10 kb in length.

In some embodiments, the insertion is or comprises a hairpin. For example, a reverse transcriptase may transcribe the hairpin, which can be incorporated into a target nucleic acid. In other embodiments, the reverse transcription template sequence includes a hairpin structure and a reverse transcriptase stops transcribing the reverse transcription template sequence at the hairpin.

In some embodiments, the edit occurs within about 500 nucleotides of a Type V PAM sequence (e.g., 5′-NTTN-3′ for a Cas12i polypeptide). In some embodiments, the edit occurs adjacent to a PAM sequence, e.g., within about 500 nucleotides upstream or downstream of a PAM sequence. In some embodiments, the edit occurs within about 400 nucleotides of a PAM sequence. In some embodiments, the edit occurs within about 400 nucleotides upstream or downstream of a PAM sequence. In some embodiments, the edit occurs within about 300 nucleotides of a PAM sequence. In some embodiments, the edit occurs within about 300 nucleotides upstream or downstream of a PAM sequence. In some embodiments, the edit occurs within about 200 nucleotides of a PAM sequence. In some embodiments, the edit occurs within about 200 nucleotides upstream or downstream of a PAM sequence. In some embodiments, the edit occurs within about 100 nucleotides of a PAM sequence. In some embodiments, the edit occurs within about 100 nucleotides upstream or downstream of a PAM sequence. In some embodiments, the edit occurs within about 50 nucleotides of a PAM sequence. In some embodiments, the edit occurs within about 50 nucleotides upstream or downstream of a PAM sequence. In some embodiments, the edit occurs within about 30 nucleotides of a PAM sequence. In some embodiments, the edit occurs within about 30 nucleotides upstream or downstream of a PAM sequence. In some embodiments, the edit occurs within about 20 nucleotides of a PAM sequence. In some embodiments, the edit occurs within about 20 nucleotides upstream or downstream of a PAM sequence.

In some embodiments, the edit starts within about 300 nucleotides upstream of the PAM sequence. In some embodiments, the edit starts within about 290 nucleotides upstream of the PAM sequence. In some embodiments, the edit starts within about 280 nucleotides upstream of the PAM sequence. In some embodiments, the edit starts within about 270 nucleotides upstream of the PAM sequence. In some embodiments, the edit starts within about 260 nucleotides upstream of the PAM sequence. In some embodiments, the edit starts within about 250 nucleotides upstream of the PAM sequence. In some embodiments, the edit starts within about 240 nucleotides upstream of the PAM sequence. In some embodiments, the edit starts within about 230 nucleotides upstream of the PAM sequence. In some embodiments, the edit starts within about 2020 nucleotides upstream of the PAM sequence. In some embodiments, the edit starts within about 210 nucleotides upstream of the PAM sequence. In some embodiments, the edit starts within about 200 nucleotides upstream of the PAM sequence. In some embodiments, the edit starts within about 190 nucleotides upstream of the PAM sequence. In some embodiments, the edit starts within about 180 nucleotides upstream of the PAM sequence. In some embodiments, the edit starts within about 170 nucleotides upstream of the PAM sequence. In some embodiments, the edit starts within about 160 nucleotides upstream of the PAM sequence. In some embodiments, the edit starts within about 150 nucleotides upstream of the PAM sequence. In some embodiments, the edit starts within about 140 nucleotides upstream of the PAM sequence. In some embodiments, the edit starts within about 130 nucleotides upstream of the PAM sequence. In some embodiments, the edit starts within about 120 nucleotides upstream of the PAM sequence. In some embodiments, the edit starts within about 110 nucleotides upstream of the PAM sequence. In some embodiments, the edit starts within about 100 nucleotides upstream of the PAM sequence. In some embodiments, the edit starts within about 90 nucleotides upstream of the PAM sequence. In some embodiments, the edit starts within about 80 nucleotides upstream of the PAM sequence. In some embodiments, the edit starts within about 70 nucleotides upstream of the PAM sequence. In some embodiments, the edit starts within about 60 nucleotides upstream of the PAM sequence. In some embodiments, the edit starts within about 50 nucleotides upstream of the PAM sequence. In some embodiments, the edit starts within about 40 nucleotides upstream of the PAM sequence. In some embodiments, the edit starts within about 30 nucleotides upstream of the PAM sequence. In some embodiments, the edit starts within about 20 nucleotides upstream of the PAM sequence. In some embodiments, the edit starts within about 10 nucleotides upstream of the PAM sequence. In some embodiments, the edit starts within about 9 nucleotides upstream of the PAM sequence. In some embodiments, the edit starts within about 8 nucleotides upstream of the PAM sequence. In some embodiments, the edit starts within about 7 nucleotides upstream of the PAM sequence. In some embodiments, the edit starts within about 6 nucleotides upstream of the PAM sequence. In some embodiments, the edit starts within about 5 nucleotides upstream of the PAM sequence. In some embodiments, the edit starts within about 4 nucleotides upstream of the PAM sequence. In some embodiments, the edit starts within about 3 nucleotides upstream of the PAM sequence. In some embodiments, the edit starts within about 2 nucleotides upstream of the PAM sequence. In some embodiments, the edit starts within about 1 nucleotide upstream of the PAM sequence.

In some embodiments, the edit starts at the PAM sequence. In some embodiments, the edit starts within about 1 nucleotide downstream of the PAM. In some embodiments, the edit starts within about 2 nucleotides downstream of the PAM. In some embodiments, the edit starts within about 3 nucleotides downstream of the PAM. In some embodiments, the edit starts within about 4 nucleotides downstream of the PAM. In some embodiments, the edit starts within about 5 nucleotides downstream of the PAM. In some embodiments, the edit starts within about 6 nucleotides downstream of the PAM. In some embodiments, the edit starts within about 7 nucleotides downstream of the PAM. In some embodiments, the edit starts within about 8 nucleotides downstream of the PAM. In some embodiments, the edit starts within about 9 nucleotides downstream of the PAM. In some embodiments, the edit starts within about 10 nucleotides downstream of the PAM. In some embodiments, the edit starts within about 11 nucleotides downstream of the PAM. In some embodiments, the edit starts within about 12 nucleotides downstream of the PAM. In some embodiments, the edit starts within about 13 nucleotides downstream of the PAM. In some embodiments, the edit starts within about 14 nucleotides downstream of the PAM. In some embodiments, the edit starts within about 15 nucleotides downstream of the PAM. In some embodiments, the edit starts within about 16 nucleotides downstream of the PAM. In some embodiments, the edit starts within about 17 nucleotides downstream of the PAM. In some embodiments, the edit starts within about 18 nucleotides downstream of the PAM. In some embodiments, the edit starts within about 19 nucleotides downstream of the PAM. In some embodiments, the edit starts within about 20 nucleotides downstream of the PAM. In some embodiments, the edit starts within about 21 nucleotides downstream of the PAM. In some embodiments, the edit starts within about 22 nucleotides downstream of the PAM. In some embodiments, the edit starts within about 23 nucleotides downstream of the PAM. In some embodiments, the edit starts within about 24 nucleotides downstream of the PAM. In some embodiments, the edit starts within about 25 nucleotides downstream of the PAM. In some embodiments, the edit starts within about 26 nucleotides downstream of the PAM. In some embodiments, the edit starts within about 27 nucleotides downstream of the PAM. In some embodiments, the edit starts within about 28 nucleotides downstream of the PAM. In some embodiments, the edit starts within about 29 nucleotides downstream of the PAM. In some embodiments, the edit starts within about 30 nucleotides downstream of the PAM.

In some embodiments, the edit ends within about 300 nucleotides upstream of the PAM sequence. In some embodiments, the edit ends within about 290 nucleotides upstream of the PAM sequence. In some embodiments, the edit ends within about 280 nucleotides upstream of the PAM sequence. In some embodiments, the edit ends within about 270 nucleotides upstream of the PAM sequence. In some embodiments, the edit ends within about 260 nucleotides upstream of the PAM sequence. In some embodiments, the edit ends within about 250 nucleotides upstream of the PAM sequence. In some embodiments, the edit ends within about 240 nucleotides upstream of the PAM sequence. In some embodiments, the edit ends within about 230 nucleotides upstream of the PAM sequence. In some embodiments, the edit ends within about 2020 nucleotides upstream of the PAM sequence. In some embodiments, the edit ends within about 210 nucleotides upstream of the PAM sequence. In some embodiments, the edit ends within about 200 nucleotides upstream of the PAM sequence. In some embodiments, the edit ends within about 190 nucleotides upstream of the PAM sequence. In some embodiments, the edit ends within about 180 nucleotides upstream of the PAM sequence. In some embodiments, the edit ends within about 170 nucleotides upstream of the PAM sequence. In some embodiments, the edit ends within about 160 nucleotides upstream of the PAM sequence. In some embodiments, the edit ends within about 150 nucleotides upstream of the PAM sequence. In some embodiments, the edit ends within about 140 nucleotides upstream of the PAM sequence. In some embodiments, the edit ends within about 130 nucleotides upstream of the PAM sequence. In some embodiments, the edit ends within about 120 nucleotides upstream of the PAM sequence. In some embodiments, the edit ends within about 110 nucleotides upstream of the PAM sequence. In some embodiments, the edit ends within about 100 nucleotides upstream of the PAM sequence. In some embodiments, the edit ends within about 90 nucleotides upstream of the PAM sequence. In some embodiments, the edit ends within about 80 nucleotides upstream of the PAM sequence. In some embodiments, the edit ends within about 70 nucleotides upstream of the PAM sequence. In some embodiments, the edit ends within about 60 nucleotides upstream of the PAM sequence. In some embodiments, the edit ends within about 50 nucleotides upstream of the PAM sequence. In some embodiments, the edit ends within about 40 nucleotides upstream of the PAM sequence. In some embodiments, the edit ends within about 30 nucleotides upstream of the PAM sequence. In some embodiments, the edit ends within about 20 nucleotides upstream of the PAM sequence. In some embodiments, the edit ends within about 10 nucleotides upstream of the PAM sequence. In some embodiments, the edit ends within about 9 nucleotides upstream of the PAM sequence. In some embodiments, the edit ends within about 8 nucleotides upstream of the PAM sequence. In some embodiments, the edit ends within about 7 nucleotides upstream of the PAM sequence. In some embodiments, the edit ends within about 6 nucleotides upstream of the PAM sequence. In some embodiments, the edit ends within about 5 nucleotides upstream of the PAM sequence. In some embodiments, the edit ends within about 4 nucleotides upstream of the PAM sequence. In some embodiments, the edit ends within about 3 nucleotides upstream of the PAM sequence. In some embodiments, the edit ends within about 2 nucleotides upstream of the PAM sequence. In some embodiments, the edit ends within about 1 nucleotide upstream of the PAM sequence.

In some embodiments, the edit ends at the PAM sequence. In some embodiments, the edit ends within about 1 nucleotide downstream of the PAM. In some embodiments, the edit ends within about 2 nucleotides downstream of the PAM. In some embodiments, the edit ends within about 3 nucleotides downstream of the PAM. In some embodiments, the edit ends within about 4 nucleotides downstream of the PAM. In some embodiments, the edit ends within about 5 nucleotides downstream of the PAM. In some embodiments, the edit ends within about 6 nucleotides downstream of the PAM. In some embodiments, the edit ends within about 7 nucleotides downstream of the PAM. In some embodiments, the edit ends within about 8 nucleotides downstream of the PAM. In some embodiments, the edit ends within about 9 nucleotides downstream of the PAM. In some embodiments, the edit ends within about 10 nucleotides downstream of the PAM. In some embodiments, the edit ends within about 11 nucleotides downstream of the PAM. In some embodiments, the edit ends within about 12 nucleotides downstream of the PAM. In some embodiments, the edit ends within about 13 nucleotides downstream of the PAM. In some embodiments, the edit ends within about 14 nucleotides downstream of the PAM. In some embodiments, the edit ends within about 15 nucleotides downstream of the PAM. In some embodiments, the edit ends within about 16 nucleotides downstream of the PAM. In some embodiments, the edit ends within about 17 nucleotides downstream of the PAM. In some embodiments, the edit ends within about 18 nucleotides downstream of the PAM. In some embodiments, the edit ends within about 19 nucleotides downstream of the PAM. In some embodiments, the edit ends within about 20 nucleotides downstream of the PAM. In some embodiments, the edit ends within about 21 nucleotides downstream of the PAM. In some embodiments, the edit ends within about 22 nucleotides downstream of the PAM. In some embodiments, the edit ends within about 23 nucleotides downstream of the PAM. In some embodiments, the edit ends within about 24 nucleotides downstream of the PAM. In some embodiments, the edit ends within about 25 nucleotides downstream of the PAM. In some embodiments, the edit ends within about 26 nucleotides downstream of the PAM. In some embodiments, the edit ends within about 27 nucleotides downstream of the PAM. In some embodiments, the edit ends within about 28 nucleotides downstream of the PAM. In some embodiments, the edit ends within about 29 nucleotides downstream of the PAM. In some embodiments, the edit ends within about 30 nucleotides downstream of the PAM.

### C. Gene Editing in Cells

In some aspects, provided herein are methods for editing a genomic site of interest (e.g., a target gene as disclosed herein) in cells using a suitable gene editing system as also disclosed herein. To perform this method, the gene editing system can be delivered to or introduced into a population of cells. In some instances, cells comprising the desired genetic editing may be collected and optionally cultured and expanded in vitro.

The cell described herein can be a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a primate cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, HEK293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a mesenchymal stem cell. In some embodiments, the cell is an embryonic stem cell. In some embodiments, the cell is a hematopoietic stem cell. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a glial cell. In some embodiments, the cell is a pancreatic islet cell, including an alpha cell, beta cell, delta cell, or enterochromaffin cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or primate cell or a murine cell. In some embodiments, the murine cell is derived from a wild-type mouse, an immunosuppressed mouse, or a disease-specific mouse model. In some embodiments, the cell is a cell within a living tissue, organ, or organism.

In some embodiments, the cell is a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g., normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

In embodiments wherein a gene editing system disclosed herein is introduced into a plurality of cells, at least about 0.5% of the cells comprise the desired edit. In some embodiments, at least about 1% of the cells comprise the desired edit. In some embodiments, at least about 2% of the cells comprise the desired edit. In some embodiments, at least about 3% of the cells comprise the desired edit. In some embodiments, at least about 4% of the cells comprise the desired edit. In some embodiments, at least about 5% of the cells comprise the desired edit. In some embodiments, at least about 10% of the cells comprise the desired edit. In some embodiments, at least about 20% of the cells comprise the desired edit. In some embodiments, at least about 30% of the cells comprise the desired edit. In some embodiments, at least about 40% of the cells comprise the desired edit. In some embodiments, at least about 50% of the cells comprise the desired edit.

The cells carrying the desired genetic edit, e.g., produced by the method disclosed herein using any of the gene editing systems also disclosed herein, are also within the scope of the present disclosure. In some instances, the cells modified by a CRISPR nuclease, reverse transcriptase, and editing template RNA as described herein may be useful as an expression system to manufacture biomolecules. For example, the modified cells may be useful to produce biomolecules such as proteins (e.g., cytokines, antibodies, antibody-based molecules), peptides, lipids, carbohydrates, nucleic acids, amino acids, and vitamins. In other embodiments, the modified cell may be useful in the production of a viral vector such as a lentivirus, adenovirus, adeno-associated virus, and oncolytic virus vector. In some embodiments, the modified cell may be useful in cytotoxicity studies. In some embodiments, the modified cell may be useful as a disease model. In some embodiments, the modified cell may be useful in vaccine production. In some embodiments, the modified cell may be useful in therapeutics. For example, in some embodiments, the modified cell may be useful in cellular therapies such as transfusions and transplantations.

In some embodiments, the cells modified by a Type V CRISPR nuclease, reverse transcriptase, and editing template RNA as described herein may be useful to establish a new cell line comprising a modified genomic sequence. In some embodiments, a modified cell of the disclosure is a modified stem cell (e.g., a modified totipotent/omnipotent stem cell, a modified pluripotent stem cell, a modified multipotent stem cell, a modified oligopotent stem cell, or a modified unipotent stem cell) that differentiates into one or more cell lineages comprising the deletion of the modified stem cell. The disclosure further provides organisms (such as animals, plants, or fungi) comprising or produced from a modified cell of the disclosure.

### D. Delivery of Gene Editing Systems to Cells

In some embodiments, any of the gene editing systems or components thereof may be formulated, for example, including a carrier, such as a carrier and/or a polymeric carrier, e.g., a liposome or lipid nanoparticle, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the Type V CRISPR nuclease, reverse transcriptase, editing template RNA (e.g., an engineered RNA guide and RT donor RNA), etc.), one or more transcripts thereof, and/or a pre-formed ribonucleoprotein to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnetofection or magnet assisted transfection, particle bombardment; and hybrid methods, such as nucleofection. In some embodiments, the present application further provides cells produced by such methods, and organisms (such as animals, plants, or fungi) comprising or produced from such cells. In some embodiments, a composition of the present invention is further delivered with an agent (e.g., compound, molecule, or biomolecule) that affects DNA repair or DNA repair machinery. In some embodiments, a composition of the present invention is further delivered with an agent (e.g., compound, molecule, or biomolecule) that affects the cell cycle.

In some embodiments, a first composition comprising a Type V CRISPR nuclease or a Type V CRISPR nuclease and a reverse transcriptase (e.g., a Type V CRISPR nuclease-reverse transcriptase fusion) is delivered to a cell. In some embodiments, a second composition comprising an engineered RNA guide or an engineered RNA guide and RT donor RNA (e.g., an editing template RNA) is delivered to a cell. In some embodiments, the first composition is contacted with a cell before the second composition is contacted with the cell. In some embodiments, the first composition is contacted with a cell at the same time as the second composition is contacted with the cell. In some embodiments, the first composition is contacted with a cell after the second composition is contacted with the cell. In some embodiments, the first composition is delivered by a first delivery method and the second composition is delivered by a second delivery method. In some embodiments, the first delivery method is the same as the second delivery method. For example, in some embodiments, the first composition and the second composition are delivered via viral delivery. In some embodiments, the first delivery method is different than the second delivery method. For example, in some embodiments, the first composition is delivered by viral delivery and the second composition is delivered by lipid nanoparticle-mediated transfer and the second composition is delivered by viral delivery or the first composition is delivered by lipid nanoparticle-mediated transfer and the second composition is delivered by viral delivery.

### IV. Therapeutic Applications

Any of the gene editing systems or modified cells generated using such a gene editing system as disclosed herein may be used for treating a disease that may be benefit from the gene edit introduced by the gene editing system or carried by the modified cells. For example, the disease may be a genetic disease and the gene edit fixes the gene mutation associated with the genetic disease. Alternatively, the disease may be associated with abnormal expression of a gene and the gene edit rescues such abnormal expression.

In some embodiments, provided herein is a method for treating a disease comprising administering to a subject (e.g., a human patient) in need of the treatment any of the gene editing system disclosed herein. The gene editing system may be delivered to a specific tissue or specific type of cells where the gene edit is needed. The gene editing system may comprise LNPs encompassing one or more of the components, one or more vectors (e.g., viral vectors) encoding one or more of the components, or a combination thereof. Components of the gene editing system may be formulated to form a pharmaceutical composition, which may further comprise one or more pharmaceutically acceptable carriers.

In some embodiments, modified cells produced using any of the gene editing systems disclosed herein may be administered to a subject (e.g., a human patient) in need of the treatment. The modified cells may comprise a substitution, insertion, and/or deletion described herein. In some examples, the modified cells may include a cell line modified by a Type V CRISPR nuclease, reverse transcriptase polypeptide, and editing template RNA (e.g., RNA guide and RT donor RNA). In some instances, the modified cells may be a heterogenous population comprising cells with different types of gene edits. Alternatively, the modified cells may comprise a substantially homogenous cell population (e.g., at least 80% of the cells in the whole population) comprising one particular gene edits. In some examples, the cells can be suspended in a suitable media.

In some embodiments, provided herein is a composition comprising the gene editing system or components thereof or the modified cells. Such a composition can be a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined number of cells. The number of cells is generally equal to the dosage of the cells which would be administered to a subject or a convenient fraction of such a dosage such as, for example, one-half or one-third of such a dosage.

A formulation of a pharmaceutical composition suitable for parenteral administration may comprise the active agent (e.g., the gene editing system or components thereof or the modified cells) combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### V. Kits and Uses Thereof

The present disclosure also provides kits or systems that can be used, for example, to carry out a method described herein. In some embodiments, the kits or systems include a Type V CRISPR nuclease and optionally a reverse transcriptase. In some embodiments, the kits or systems include a polynucleotide that encodes a Type V CRISPR nuclease and reverse transcriptase, and optionally the polynucleotide is comprised within a vector, e.g., as described herein. In some embodiments, the kits or systems include a Type V nuclease-reverse transcriptase fusion polypeptide. The kits or systems also can include a reverse transcriptase, and an editing template RNA (e.g., an engineered RNA guide and RT donor RNA) as described herein. The gRNA and/or RT donor RNA of the kits or systems of the invention can be designed to target a sequence of interest. The Type V CRISPR nuclease, reverse transcriptase, and editing template RNA (e.g., an engineered RNA guide and RT donor RNA) can be packaged within the same vial or other vessel within a kit or system or can be packaged in separate vials or other vessels, the contents of which can be mixed prior to use. The kits or systems can additionally include, optionally, a buffer and/or instructions for use of a Type V CRISPR nuclease and reverse transcriptase, along with the editing template RNA (e.g., an engineered RNA guide and RT donor RNA).

In some embodiments, the kit comprises a first composition comprising a Type V CRISPR nuclease or a Type V CRISPR nuclease and a reverse transcriptase (e.g., a Type V CRISPR nuclease-reverse transcriptase fusion). In some embodiments, the kit comprises a second composition comprising an engineered RNA guide or an engineered RNA guide and RT donor RNA (e.g., an editing template RNA). In some embodiments, the first composition and the second composition are packaged within the same vial. In some embodiments, the first composition and the second composition are packaged within different vials.

In some embodiments, the kit may be useful for research purposes. For example, in some embodiments, the kit may be useful to study gene function.

All references and publications cited herein are hereby incorporated by reference.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Editing in HEK293T Cells Using Engineered sgRNA Sequences

This Example describes an indel assessment on a mammalian target by the nuclease of SEQ ID NO: 1 and engineered sgRNA sequences introduced into HEK293T cells by transient transfection.

Nucleic acids encoding either the nuclease or the nuclease fused to a reverse transcriptase, as shown in Table 1, were individually cloned into pcda3.1 backbones (Invitrogen™). The plasmids were then maxi-prepped and diluted. The sgRNA sequences comprising scaffold sequences set forth in Table 2 were further individually cloned into pUC19 backbones (New England Biolabs®) under a U6 promoter, purified, and diluted. A spacer targeting a genomic site of interest can be located at the 3′ end of the scaffold sequence (as indicated in Table 2 above) to form an sgRNA molecule. The tested EMX1 target sequence was TGTTGCCCTCATAACTTATC (SEQ ID NO: 2) and had a 5′-TTTC-3′ PAM sequence; the spacer sequence used at the 3′ end of the scaffold sequences was UGUUGCCCUCAUAACUUAUC (SEQ ID NO: 3).

TABLE 1
Nuclease and Nuclease-RT Fusion Polypeptide Sequences

| Full | | SEQ ID |
|---|---|---|
| Description | Sequence | NO |
| CRISPR | MEVQYNKIDVYPMKIRLYPTKEQAKTIDGWLMGLQKAYNMTL | 1 |
| Nuclease | YALKEGVPELRQKSKSGDAEFPNWKYIGKREWLDELRKNSPCV | |
| | ANVPGGCLSSTVGGALGADMRKAWESQGKLPVDAWFRATDAN | |
| | GRHIVRWYSDAKPRKSCFFQIEAGKISREKQSVYITLRKDFRVKA | |
| | RGWNDKIRFSEESDEGFFEKYRDSKKVLSLRVSRDNCGDYFATI | |
| | TLKDVYRPTKVEAERKGVGIDVGVRAMATDSDGRVYENPRIKQ | |
| | KFEDKKTELGRQLSRRYGAKNKQFRQDCKEARSFNRLHESEINE | |
| | KLVVPKTVLPSRRYNQAQLKLSKLERKMRRKRDMAQHVYSAE | |
| | VVRKASLVAIENLNVKGMMADSNLADRLSDAAMSELLRKLKY | |
| | KAEWSGASYHAIGTFTASTQRCAKCGYILRGENKLARDDSVFVC | |
| | PVCRNVDGRDANAAKSILQVAQKEISEGVPSADLIKKLEVKKTK | |
| | TYPDKPIGKAFPNVFTHFSEEYKKQKRNPFVIVDENKNVLNDAQ | |
| | GYGYRNRQSAQKFWAHKMNQ | |
| mh6-nuclease- | MKIEEGKGHHHHHHMEVQYNKIDVYPMKIRLYPTKEQAKTIDG | 4 |
| NLS | WLMGLQKAYNMTLYALKEGVPELRQKSKSGDAEFPNWKYIGK | |
| | REWLDELRKNSPCVANVPGGCLSSTVGGALGADMRKAWESQG | |
| | KLPVDAWFRATDANGRHIVRWYSDAKPRKSCFFQIEAGKISREK | |
| | QSVYITLRKDFRVKARGWNDKIRFSEESDEGFFEKYRDSKKVLS | |
| | LRVSRDNCGDYFATITLKDVYRPTKVEAERKGVGIDVGVRAMA | |
| | TDSDGRVYENPRIKQKFEDKKTELGRQLSRRYGAKNKQFRQDC | |
| | KEARSFNRLHESEINEKLVVPKTVLPSRRYNQAQLKLSKLERKM | |
| | RRKRDMAQHVYSAEVVRKASLVAIENLNVKGMMADSNLADRL | |
| | SDAAMSELLRKLKYKAEWSGASYHAIGTFTASTQRCAKCGYILR | |
| | GENKLARDDSVFVCPVCRNVDGRDANAAKSILQVAQKEISEGVP | |
| | SADLIKKLEVKKTKTYPDKPIGKAFPNVFTHFSEEYKKQKRNPFV | |
| | IVDENKNVLNDAQGYGYRNRQSAQKFWAHKMNQKRPAATKKA | |
| | GQAKKKK | |
| mh6-nuclease- | MKIEEGKGHHHHHHMEVQYNKIDVYPMKIRLYPTKEQAKTIDG | 5 |
| NLS-2xSGGS- | WLMGLQKAYNMTLYALKEGVPELRQKSKSGDAEFPNWKYIGK | |
| XTEN (16 aa)- | REWLDELRKNSPCVANVPGGCLSSTVGGALGADMRKAWESQG | |
| 2xSGGS- | KLPVDAWFRATDANGRHIVRWYSDAKPRKSCFFQIEAGKISREK | |
| mutMMLVRT- | QSVYITLRKDFRVKARGWNDKIRFSEESDEGFFEKYRDSKKVLS | |
| NLS | LRVSRDNCGDYFATITLKDVYRPTKVEAERKGVGIDVGVRAMA | |
| | TDSDGRVYENPRIKQKFEDKKTELGRQLSRRYGAKNKQFRQDC | |
| | KEARSFNRLHESEINEKLVVPKTVLPSRRYNQAQLKLSKLERKM | |
| | RRKRDMAQHVYSAEVVRKASLVAIENLNVKGMMADSNLADRL | |
| | SDAAMSELLRKLKYKAEWSGASYHAIGTFTASTQRCAKCGYILR | |
| | GENKLARDDSVFVCPVCRNVDGRDANAAKSILQVAQKEISEGVP | |
| | SADLIKKLEVKKTKTYPDKPIGKAFPNVFTHFSEEYKKQKRNPFV | |
| | IVDENKNVLNDAQGYGYRNRQSAQKFWAHKMNQKRPAATKK | |
| | AGQAKKKKSGGSSGGSSGSETPGTSESATPESSGGSSGGSTLNIE | |
| | DEYRLHETSKEPDVSLGSTWLSDFPQAWAETGGMGLAVRQAPL | |
| | IIPLKATSTPVSIKQYPMSQEARLGIKPHIQRLLDQGILVPCQSPW | |
| | NTPLLPVKKPGTNDYRPVQDLREVNKRVEDIHPTVPNPYNLLSG | |
| | LPPSHQWYTVLDLKDAFFCLRLHPTSQPLFAFEWRDPEMGISGQ | |
| | LTWTRLPQGFKNSPTLFNEALHRDLADFRIQHPDLILLQYVDDLL | |
| | LAATSELDCQQGTRALLQTLGNLGYRASAKKAQICQKQVKYLG | |
| | YLLKEGQRWLTEARKETVMGQPTPKTPRQLREFLGKAGFCRLFI | |
| | PGFAEMAAPLYPLTKPGTLFNWGPDQQKAYQEIKQALLTAPAL | |
| | GLPDLTKPFELFVDEKQGYAKGVLTQKLGPWRRPVAYLSKKLD | |
| | PVAAGWPPCLRMVAAIAVLTKDAGKLTMGQPLVILAPHAVEAL | |
| | VKQPPDRWLSNARMTHYQALLLDTDRVQFGPVVALNPATLLPL | |
| | PEEGLOHNCLDILAEAHGTRPDLTDQPLPDADHTWYTDGSSLLQ | |
| | EGQRKAGAAVTTETEVIWAKALPAGTSAQRAELIALTQALKMA | |
| | EGKKLNVYTDSRYAFATAHIHGEIYRRRGWLTSEGKEIKNKDEI | |
| | LALLKALFLPKRLSIIHCPGHQKGHSAEARGNRMADQAARKAAI | |
| | TETPDTSTLLIENSSPMKRTADGSEFESPKKKRKV | |

TABLE 2
Parent and Engineered Scaffold Sequences

| | | SEQ |
|---|---|---|
| sgRNA Reference | Scaffold Sequences | ID NO |
| Parent | GUGAGGUGCAGCCACGAGGUGCGAAUAGGAAGUACGCAG | 71 |
| | CAAUGUGCUGAAUCGUUCGCACGAAAAUUGGCAUUUUUG | |
| | AAAACCAAAGCCAAUAAUCAUAUCCAUAAAUAUGUGUGG | |
| | UUAUGAACUGCAAG | |
| 5truncate_4 | GGUGCAGCCACGAGGUGCGAAUAGGAAGUACGCAGCAAU | 6 |
| | GUGCUGAAUCGUUCGCACGAAAAUUGGCAUUAUUGAAAA | |
| | CCAAUGCCAAUAAUCAUAUCCAUAAAUAUGUGUGGUUAU | |
| | GAACUGCAAG | |
| 5truncate_6 | UGCAGCCACGAGGUGCGAAUAGGAAGUACGCAGCAAUGU | 7 |
| | GCUGAAUCGUUCGCACGAAAAUUGGCAUUAUUGAAAACC | |
| | AAUGCCAAUAAUCAUAUCCAUAAAUAUGUGUGGUUAUGA | |
| | ACUGCAAG | |
| 5truncate_9 | AGCCACGAGGUGCGAAUAGGAAGUACGCAGCAAUGUGCU | 8 |
| | GAAUCGUUCGCACGAAAAUUGGCAUUAUUGAAAACCAAU | |
| | GCCAAUAAUCAUAUCCAUAAAUAUGUGUGGUUAUGAACU | |
| | GCAAG | |
| 5truncate_10 | GCCACGAGGUGCGAAUAGGAAGUACGCAGCAAUGUGCUG | 9 |
| | AAUCGUUCGCACGAAAAUUGGCAUUAUUGAAAACCAAUG | |
| | CCAAUAAUCAUAUCCAUAAAUAUGUGUGGUUAUGAACUG | |
| | CAAG | |
| 5truncate_11 | CCACGAGGUGCGAAUAGGAAGUACGCAGCAAUGUGCUGA | 10 |
| | AUCGUUCGCACGAAAAUUGGCAUUAUUGAAAACCAAUGC | |
| | CAAUAAUCAUAUCCAUAAAUAUGUGUGGUUAUGAACUGC | |
| | AAG | |
| 5truncate_12 | CACGAGGUGCGAAUAGGAAGUACGCAGCAAUGUGCUGAA | 11 |
| | UCGUUCGCACGAAAAUUGGCAUUAUUGAAAACCAAUGCC | |
| | AAUAAUCAUAUCCAUAAAUAUGUGUGGUUAUGAACUGCA | |
| | AG | |
| 5truncate_13 | ACGAGGUGCGAAUAGGAAGUACGCAGCAAUGUGCUGAAU | 12 |
| | CGUUCGCACGAAAAUUGGCAUUAUUGAAAACCAAUGCCA | |
| | AUAAUCAUAUCCAUAAAUAUGUGUGGUUAUGAACUGCAA | |
| | G | |
| 5truncate_14 | CGAGGUGCGAAUAGGAAGUACGCAGCAAUGUGCUGAAUC | 13 |
| | GUUCGCACGAAAAUUGGCAUUAUUGAAAACCAAUGCCAA | |
| | UAAUCAUAUCCAUAAAUAUGUGUGGUUAUGAACUGCAAG | |
| 5truncate_15 | GAGGUGCGAAUAGGAAGUACGCAGCAAUGUGCUGAAUCG | 14 |
| | UUCGCACGAAAAUUGGCAUUAUUGAAAACCAAUGCCAAU | |
| | AAUCAUAUCCAUAAAUAUGUGUGGUUAUGAACUGCAAG | |
| 5truncate_16 | AGGUGCGAAUAGGAAGUACGCAGCAAUGUGCUGAAUCGU | 15 |
| | UCGCACGAAAAUUGGCAUUAUUGAAAACCAAUGCCAAUA | |
| | AUCAUAUCCAUAAAUAUGUGUGGUUAUGAACUGCAAG | |
| 5truncate_17 | GGUGCGAAUAGGAAGUACGCAGCAAUGUGCUGAAUCGUU | 16 |
| | CGCACGAAAAUUGGCAUUAUUGAAAACCAAUGCCAAUAA | |
| | UCAUAUCCAUAAAUAUGUGUGGUUAUGAACUGCAAG | |
| 5truncate_19 | UGCGAAUAGGAAGUACGCAGCAAUGUGCUGAAUCGUUCG | 17 |
| | CACGAAAAUUGGCAUUAUUGAAAACCAAUGCCAAUAAUC | |
| | AUAUCCAUAAAUAUGUGUGGUUAUGAACUGCAAG | |
| 5truncate_20 | GCGAAUAGGAAGUACGCAGCAAUGUGCUGAAUCGUUCGC | 18 |
| | ACGAAAAUUGGCAUUAUUGAAAACCAAUGCCAAUAAUCA | |
| | UAUCCAUAAAUAUGUGUGGUUAUGAACUGCAAG | |
| StemLoopTrunc_1 | GUGAGGUGCAGCCACGAGGUGCGAAUAGGAAGUACGCAG | 19 |
| | CGAAAGCUGAAUCGUUCGCACGAAAAUUGGCAUUAUUGA | |
| | AAACCAAUGCCAAUAAUCAUAUCCAUAAAUAUGUGUGGU | |
| | UAUGAACUGCAAG | |
| StemLoopTrunc_2 | GUGAGGUGCAGCCACGAGGUGCGAAUGAAGUACGCAGCA | 20 |
| | AUGUGCUGAAUCGUUCGCACGAAAAUUGGCAUUAUUGAA | |
| | AACCAAUGCCAAUAAUCAUAUCCAUAAAUAUGUGUGGUU | |
| | AUGAACUGCAAG | |
| StemLoopTrunc_3 | GUGAGGUGCAGCCACGAGGUGCGAAUAGGAAGUACGCAG | 21 |
| | CAAUGUGCUGGAAAUCGUUCGCACGAAAAUUGGCAUUAU | |
| | UGAAAACCAAUGCCAAUAAUCAUAUCCAUAAAUAUGUGU | |
| | GGUUAUGAACUGCAAG | |
| StemLoopTrunc_4 | GUGAGGUGCAGCCACGAGGUGCGAAUAGGAAGUACGCAG | 22 |
| | AAUGUCUGAAUCGUUCGCACGAAAAUUGGCAUUAUUGAA | |
| | AACCAAUGCCAAUAAUCAUAUCCAUAAAUAUGUGUGGUU | |
| | AUGAACUGCAAG | |
| StemLoopTrunc_5 | GUGAGGUGCAGCCACGAGGUGCGAAUAGGAAGUACGCGA | 23 |
| | AUGUCGAAUCGUUCGCACGAAAAUUGGCAUUAUUGAAAA | |
| | CCAAUGCCAAUAAUCAUAUCCAUAAAUAUGUGUGGUUAU | |
| | GAACUGCAAG | |
| StemLoopTrunc_8 | GUGAGGUGCAGCCACGAGGUGCGAAUAGGAGAAAUCGUU | 24 |
| | CGCACGAAAAUUGGCAUUAUUGAAAACCAAUGCCAAUAA | |
| | UCAUAUCCAUAAAUAUGUGUGGUUAUGAACUGCAAG | |
| | [spacer] | |
| StemLoopTrunc_9 | GUGAGGUGCAGCCACGAGGUGCGAUAGGAAGUACGCAGC | 25 |
| | AAUGUGCUGAAUCGUCGCACGAAAAUUGGCAUUAUUGAA | |
| | AACCAAUGCCAAUAAUCAUAUCCAUAAAUAUGUGUGGUU | |
| | AUGAACUGCAAG | |
| StemLoopTrunc_10 | GUGAGGUGCAGCCACGAGGUGCGUAGGAAGUACGCAGCA | 26 |
| | AUGUGCUGAAUCGCGCACGAAAAUUGGCAUUAUUGAAAA | |
| | CCAAUGCCAAUAAUCAUAUCCAUAAAUAUGUGUGGUUAU | |
| | GAACUGCAAG | |
| StemLoopTrunc_11 | GUGAGGUGCAGCCACGAGGUGCGAAUAGGAGUACGCAGC | 27 |
| | AAUGUGCUGAACGUUCGCACGAAAAUUGGCAUUAUUGAA | |
| | AACCAAUGCCAAUAAUCAUAUCCAUAAAUAUGUGUGGUU | |
| | AUGAACUGCAAG | |
| StemLoopTrunc_13 | GUGAGGUGCAGCCACGAGGUGCGAAAGUACGCAGCAAUG | 28 |
| | UGCUGAAUUCGCACGAAAAUUGGCAUUAUUGAAAACCAA | |
| | UGCCAAUAAUCAUAUCCAUAAAUAUGUGUGGUUAUGAAC | |
| | UGCAAG | |
| StemLoopTrunc_14 | GUGAGGUGCAGCCACGAGGGUGCGAAUCUAAAGUGGACA | 29 |
| | GAAAUGGGAACAGACGUUCGCACCGAAAAUUGGCAUUAU | |
| | UGAAAACCAAUGCCAAUAAUCAUAUCCAUAAAUAUGUGU | |
| | GGUUAUGAACUGCAAG | |
| StemLoopTrunc_15 | GUGAGGUGCAGCCACGAGGUGCGAAUAGGAAGUACGCAG | 30 |
| | CAAUGUGCUGAAUCGUUCGCACGAAAAUUGGCAUUAUUG | |
| | GAAACAAUGCCAAUAAUCAUAUCCAUAAAUAUGUGUGGU | |
| | UAUGAACUGCAAG | |
| StemLoopTrunc_17 | GUGAGGUGCAGCCACGAGGUGCGAAUAGGAAGUACGCAG | 31 |
| | CAAUGUGCUGAAUCGUUCGCACGAAAAUUGGCAUUAUGA | |
| | AAACCAUGCCAAUAAUCAUAUCCAUAAAUAUGUGUGGUU | |
| | AUGAACUGCAAG | |
| StemLoopTrunc_18 | GUGAGGUGCAGCCACGAGGUGCGAAUAGGAAGUACGCAG | 32 |
| | CAAUGUGCUGAAUCGUUCGCACGAAAAUUGGCAUUAGAA | |
| | AACCUGCCAAUAAUCAUAUCCAUAAAUAUGUGUGGUUAU | |
| | GAACUGCAAG | |
| StemLoopTrunc_20 | GUGAGGUGCAGCCACGAGGUGCGAAUAGGAAGUACGCAG | 33 |
| | CAAUGUGCUGAAUCGUUCGCACGAAAAUUGGCAGAAAAC | |
| | CUGCCAAUAAUCAUAUCCAUAAAUAUGUGUGGUUAUGAA | |
| | CUGCAAG | |
| StemLoopTrunc_21 | GUGAGGUGCAGCCACGAGGUGCGAAUAGGAAGUACGCAG | 34 |
| | CAAUGUGCUGAAUCGUUCGCACGAAAAUUGGAUUAUUGA | |
| | AAACCAAUCCAAUAAUCAUAUCCAUAAAUAUGUGUGGUU | |
| | AUGAACUGCAAG | |
| StemLoopTrunc_23 | GUGAGGUGCAGCCACGAGGUGCGAAUAGGAAGUACGCAG | 35 |
| | CAAUGUGCUGAAUCGUUCGCACGAAAGUUGGCGGAUUUC | |
| | UUCGGAAAUCGAGCCAAUAAUCAUAUCCAUAAAUAUGUG | |
| | UGGUUAUGAACUGCAAG | |
| StemLoopTrunc_24 | GUGAGGUGCAGCCACGAGGUGCGAAUAGGAAGUACGCAG | 36 |
| | CAAUGUGCUGAAUCGUUCGCACGAAAGUUGGCGGAUUUC | |
| | UUCGGAAAUCGAGCCAACAAUCAUAUCCAUAAAUAUGUG | |
| | UGGUUAUGAACUGCAAG | |
| StemLoopTrunc_25 | GUGAGGUGCAGCCACGAGGUGCGAAUAGGAAGUACGCAG | 37 |
| | CAAUGUGCUGAAUCGUUCGCACGAAAAUUGGCAUUAUUG | |
| | AAAACCAAUGCCAAUAAUCAUAUCCAUAGAAAUGUGGUU | |
| | AUGAACUGCAAG | |
| StemLoopTrunc_26 | GUGAGGUGCAGCCACGAGGUGCGAAUAGGAAGUACGCAG | 38 |
| | CAAUGUGCUGAAUCGUUCGCACGAAAAUUGGCAUUAUUG | |
| | AAAACCAAUGCCAAUAAUCAUAUCCAAAAUAUGUUGGUU | |
| | AUGAACUGCAAG | |
| StemLoopTrunc_27 | GUGAGGUGCAGCCACGAGGUGCGAAUAGGAAGUACGCAG | 39 |
| | CAAUGUGCUGAAUCGUUCGCACGAAAAUUGGCAUUAUUG | |
| | AAAACCAAUGCCAAUAAUCAUAUCCAAAUAUGUGGUUAU | |
| | GAACUGCAAG | |
| StemLoopTrunc_28 | GUGAGGUGCAGCCACGAGGUGCGAAUAGGAAGUACGCAG | 40 |
| | CAAUGUGCUGAAUCGUUCGCACGAAAAUUGGCAUUAUUG | |
| | AAAACCAAUGCCAAUAAUCAUAUCAAAUAUGUGUUAUGA | |
| | ACUGCAAG | |
| StemLoopTrunc_29 | GUGAGGUGCAGCCACGAGGUGCGAAUAGGAAGUACGCAG | 41 |
| | CAAUGUGCUGAAUCGUUCGCACGAAAAUUGGCAUUAUUG | |
| | AAAACCAAUGCCAAUAAUCAUAUAAAUAUGUUUAUGAAC | |
| | UGCAAG | |
| StemLoopTrunc_31 | GUGAGGUGCAGCCACGAGGUGCGAAUAGGAAGUACGCAG | 42 |
| | CAAUGUGCUGAAUCGUUCGCACGAAAAUUGGCAUUAUUG | |
| | AAAACCAAUGCCAAUAAUCAUAAAAUAUGUUAUGAACUG | |
| | CAAG | |
| StemLoopTrunc_32 | GUGAGGUGCAGCCACGAGGUGCGAAUAGGAAGUACGCAG | 43 |
| | CAAUGUGCUGAAUCGUUCGCACGAAAAUUGGCAUUAUUG | |
| | AAAACCAAUGCCAAUAAUCAUAAAUAUGUAUGAACUGCA | |
| | AG | |
| StemLoopTrunc_33 | GUGAGGUGCAGCCACGAGGUGCGAAUAGGAAGUACGCAG | 44 |
| | CAAUGUGCUGAAUCGUUCGCACGAAAAUUGGCAUUAUUG | |
| | AAAACCAAUGCCAAUAAUCAAAAUAUGUUGAACUGCAAG | |
| StemLoopTrunc_34 | GUGAGGUGCAGCCACGAGGUGCGAAUAGGAAGUACGCAG | 45 |
| | CAAUGUGCUGAAUCGUUCGCACGAAAAUUGGCAUUAUUG | |
| | AAAACCAAUGCCAAUAAUCAAAUAUGUGAACUGCAAG | |
| | [spacer] | |
| StemLoopTrunc_35 | GUGAGGUGCAGCCACGAGGUGCGAAUAGGAAGUACGCAG | 46 |
| | CAAUGUGCUGAAUCGUUCGCACGAAAAUUGGCAUUAUUG | |
| | AAAACCAAUGCCAAUAAUCAUAUCCAUAUUAUCUGCGGA | |
| | UGGAUGUGAACUGCAAG | |
| StemLoopTrunc_36 | GUGAGGUGCAGCCACGAGGUGCGAAUAGGAAGUACGCAG | 47 |
| | CAAUGUGCUGAAUCGUUCGCACGAAAAUUGGCAUUAUUG | |
| | AAAACCAAUGCCAAUAAGCAUAUCCAUAUUAUCUGCGGA | |
| | UGGAUGUGCACUGCAAG | |
| StemPairing_3 | GUGAGGUGCAGCCACGAGGUGCGACUAGGAAGUACGCAG | 48 |
| | CAAUGUGCUGAAUCGGUCGCACGAAAAUUGGCAUUAUUG | |
| | AAAACCAAUGCCAAUAAUCAUAUCCAUAAAUAUGUGUGG | |
| | UUAUGAACUGCAAG | |
| StemPairing_4 | GUGAGGUGCAGCCACGAGGUGCGAACAGGAAGUACGCAG | 49 |
| | CAAUGUGCUGAAUCGUUCGCACGAAAAUUGGCAUUAUUG | |
| | AAAACCAAUGCCAAUAAUCAUAUCCAUAAAUAUGUGUGG | |
| | UUAUGAACUGCAAG | |
| StemPairing_5 | GUGAGGUGCAGCCACGAGGUGCGAAUAGGCAGUACGCAG | 50 |
| | CAAUGUGCUGAAGCGUUCGCACGAAAAUUGGCAUUAUUG | |
| | AAAACCAAUGCCAAUAAUCAUAUCCAUAAAUAUGUGUGG | |
| | UUAUGAACUGCAAG | |
| StemPairing_6 | GUGAGGUGCAGCCACGAGGUGCGAAUAGGAAGUACGCCG | 51 |
| | CAAUGUGCGGAAUCGUUCGCACGAAAAUUGGCAUUAUUG | |
| | AAAACCAAUGCCAAUAAUCAUAUCCAUAAAUAUGUGUGG | |
| | UUAUGAACUGCAAG | |
| StemPairing_7 | GUGAGGUGCAGCCACGAGGUGCGAAUAGGAAGUACGCAG | 52 |
| | CAAUGUGCUGAAUCGUUCGCACGAAACUUGGCAUUAUUG | |
| | AAAACCAAUGCCAAGAAUCAUAUCCAUAAAUAUGUGUGG | |
| | UUAUGAACUGCAAG | |
| StemPairing_8 | GUGAGGUGCAGCCACGAGGUGCGAAUAGGAAGUACGCAG | 53 |
| | CAAUGUGCUGAAUCGUUCGCACGAAAACUGGCAUUAUUG | |
| | AAAACCAAUGCCAGUAAUCAUAUCCAUAAAUAUGUGUGG | |
| | UUAUGAACUGCAAG | |
| StemPairing_9 | GUGAGGUGCAGCCACGAGGUGCGAAUAGGAAGUACGCAG | 54 |
| | CAAUGUGCUGAAUCGUUCGCACGAAAAUCGGCAUUAUUG | |
| | AAAACCAAUGCCGAUAAUCAUAUCCAUAAAUAUGUGUGG | |
| | UUAUGAACUGCAAG | |
| StemPairing_10 | GUGAGGUGCAGCCACGAGGUGCGAAUAGGAAGUACGCAG | 55 |
| | CAAUGUGCUGAAUCGUUCGCACGAAAAUUGGCAUUCUUG | |
| | AAAACCAAGGCCAAUAAUCAUAUCCAUAAAUAUGUGUGG | |
| | UUAUGAACUGCAAG | |
| StemPairing_11 | GUGAGGUGCAGCCACGAGGUGCGAAUAGGAAGUACGCAG | 56 |
| | CAAUGUGCUGAAUCGUUCGCACGAAAAUUGGCAUUACUG | |
| | AAAACCAGUGCCAAUAAUCAUAUCCAUAAAUAUGUGUGG | |
| | UUAUGAACUGCAAG | |
| StemPairing_12 | GUGAGGUGCAGCCACGAGGUGCGAAUAGGAAGUACGCAG | 57 |
| | CAAUGUGCUGAAUCGUUCGCACGAAAAUUGGCAUUAUCG | |
| | AAAACCGAUGCCAAUAAUCAUAUCCAUAAAUAUGUGUGG | |
| | UUAUGAACUGCAAG | |
| StemPairing_13 | GUGAGGUGCAGCCACGAGGUGCGAAUAGGAAGUACGCAG | 58 |
| | CAAUGUGCUGAAUCGUUCGCACGAAAAUUGGCAUUAUUG | |
| | AAAACCAAUGCCAAUAACCAUAUCCAUAAAUAUGUGUGG | |
| | UUAUGGACUGCAAG | |
| StemPairing_14 | GUGAGGUGCAGCCACGAGGUGCGAAUAGGAAGUACGCAG | 59 |
| | CAAUGUGCUGAAUCGUUCGCACGAAAAUUGGCAUUAUUG | |
| | AAAACCAAUGCCAAUAAUCCUAUCCAUAAAUAUGUGUGG | |
| | UUAGGAACUGCAAG | |
| StemPairing_15 | GUGAGGUGCAGCCACGAGGUGCGAAUAGGAAGUACGCAG | 60 |
| | CAAUGUGCUGAAUCGUUCGCACGAAAAUUGGCAUUAUUG | |
| | AAAACCAAUGCCAAUAAUCACAUCCAUAAAUAUGUGUGG | |
| | UUGUGAACUGCAAG | |
| StemPairing_16 | GUGAGGUGCAGCCACGAGGUGCGAAUAGGAAGUACGCAG | 61 |
| | CAAUGUGCUGAAUCGUUCGCACGAAAAUUGGCAUUAUUG | |
| | AAAACCAAUGCCAAUAAUCAUCUCCAUAAAUAUGUGUGG | |
| | UGAUGAACUGCAAG | |
| StemPairing_17 | GUGAGGUGCAGCCACGAGGUGCGAAUAGGAAGUACGCAG | 62 |
| | CAAUGUGCUGAAUCGUUCGCACGAAAAUUGGCAUUAUUG | |
| | AAAACCAAUGCCAAUAAUCAUAUCCAUAAAUAUGUGUGG | |
| | AUAUGAACUGCAAG | |
| StemPairing_18 | GUGAGGUGCAGCCACGAGGUGCGAAUAGGAAGUACGCAG | 63 |
| | CAAUGUGCUGAAUCGUUCGCACGAAAAUUGGCAUUAUUG | |
| | AAAACCAAUGCCAAUAAUCAUAUCCGUAAAUAUGUGCGG | |
| | UUAUGAACUGCAAG | |
| StemPairing_19 | GUGAGGUGCAGCCACGAGGUGCGAAUAGGAAGUACGCAG | 64 |
| | CAAUGUGCUGAAUCGUUCGCACGAAAAUUGGCAUUAUUG | |
| | AAAACCAAUGCCAAUAAUCAUAUCCACAAAUAUGUGUGG | |
| | UUAUGAACUGCAAG | |
| StemPairing_20 | GUGAGGUGCAGCCACGAGGUGCGAAUAGGAAGUACGCAG | 65 |
| | CAAUGUGCUGAAUCGUUCGCACGAAAAUUGGCAUUAUUG | |
| | AAAACCAAUGCCAAUAAUCAUAUCCAUGAAUAUGCGUGG | |
| | UUAUGAACUGCAAG | |
| Selongate_1 | CGUGAGGUGCAGCCACGAGGUGCGAAUAGGAAGUACGCA | 66 |
| | GCAAUGUGCUGAAUCGUUCGCACGAAAAUUGGCAUUAUU | |
| | GAAAACCAAUGCCAAUAAUCAUAUCCAUAAAUAUGUGUG | |
| | GUUAUGAACUGCAAG | |
| Selongate_2 | UCGUGAGGUGCAGCCACGAGGUGCGAAUAGGAAGUACGC | 67 |
| | AGCAAUGUGCUGAAUCGUUCGCACGAAAAUUGGCAUUAU | |
| | UGAAAACCAAUGCCAAUAAUCAUAUCCAUAAAUAUGUGU | |
| | GGUUAUGAACUGCAAG | |
| 5elongate_3 | UUCGUGAGGUGCAGCCACGAGGUGCGAAUAGGAAGUACG | 68 |
| | CAGCAAUGUGCUGAAUCGUUCGCACGAAAAUUGGCAUUA | |
| | UUGAAAACCAAUGCCAAUAAUCAUAUCCAUAAAUAUGUG | |
| | UGGUUAUGAACUGCAAG | |
| Selongate_5 | AUUUCGUGAGGUGCAGCCACGAGGUGCGAAUAGGAAGUA | 69 |
| | CGCAGCAAUGUGCUGAAUCGUUCGCACGAAAAUUGGCAU | |
| | UAUUGAAAACCAAUGCCAAUAAUCAUAUCCAUAAAUAUG | |
| | UGUGGUUAUGAACUGCAAG | |
| 5elongate_6 | UAUUUCGUGAGGUGCAGCCACGAGGUGCGAAUAGGAAGU | 70 |
| | ACGCAGCAAUGUGCUGAAUCGUUCGCACGAAAAUUGGCA | |
| | UUAUUGAAAACCAAUGCCAAUAAUCAUAUCCAUAAAUAU | |
| | GUGUGGUUAUGAACUGCAAG | |

Approximately 16 hours prior to transfection, 25,000 HEK293T cells in DMEM/10% FBS+Pen/Strep were plated into each well of a 96-well plate. On the day of transfection, the cells were 70-90% confluent. For each well to be transfected, a mixture of Lipofectamine™ 2000 (ThermoFisher®) and Opti-MEM™ (ThermoFisher®) was prepared and then incubated at room temperature for 5-20 minutes (Solution 1). After incubation, the Lipofectamine™:OptiMEM™ mixture was added to a separate mixture containing nuclease plasmid, sgRNA, and water (Solution 2). In the case of negative controls, the sgRNA was not included in Solution 2. The solution 1 and solution 2 mixtures were mixed by pipetting up and down and then incubated at room temperature for 25 minutes. Following incubation, Solution 1 and Solution 2 mixture were added dropwise to each well of a 96 well plate containing the cells. 72 hours post transfection, cells were trypsinized by adding TrypLE™ (ThermoFisher®) to the center of each well and incubated for approximately 5 minutes. D10 media was then added to each well and mixed to resuspend cells. The cells were then spun down for 10 minutes, and the supernatant was discarded. QuickExtract™ extraction reagent (Biosearch™ Technologies) was added to ⅕ the amount of the original cell suspension volume. The resuspended cell solution was incubated at 65° C. for 15 minutes, 68° C. for 15 minutes, and 98° C. for 10 minutes.

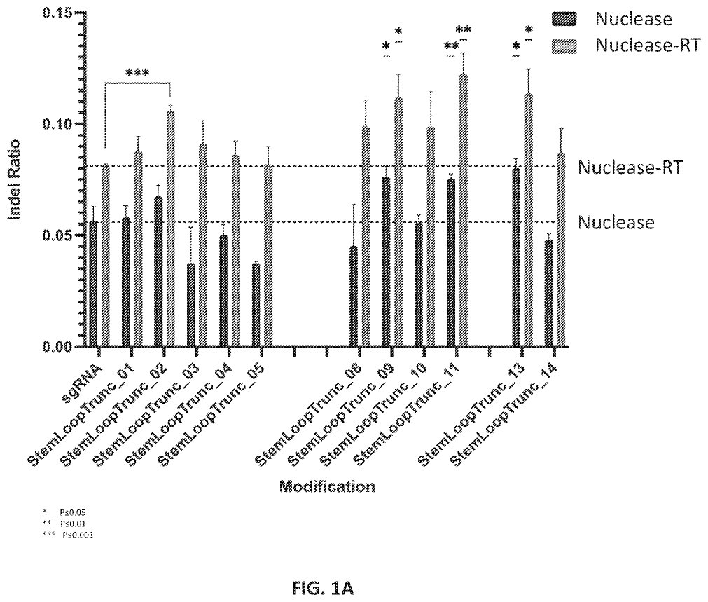

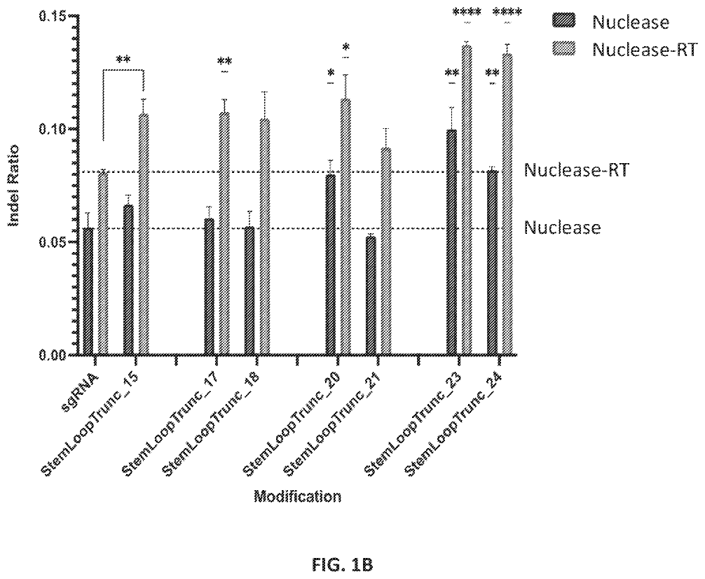

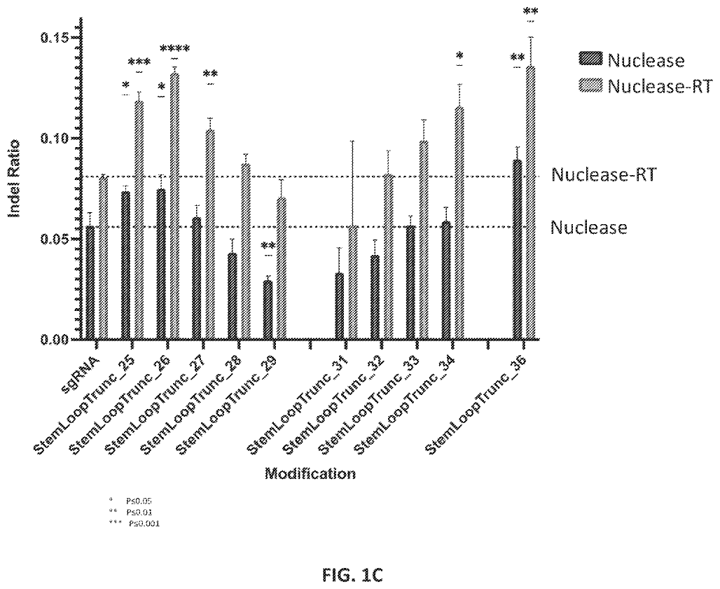

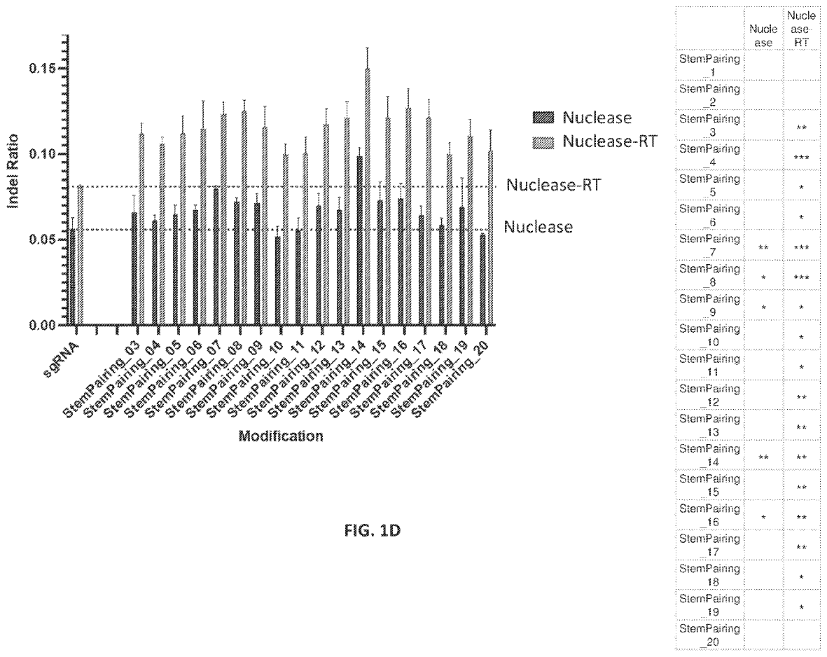

NGS samples were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit. Results are shown in FIGS. 1A-1D and summarized in Table 3. Several of the engineered sgRNAs resulted in a higher percentage of NGS reads having indels compared to the parent sgRNA control (SEQ ID NO: 71). For example, certain engineered sgRNAs (e.g., StemLoopTrunc_11, StemLoopTrunc_23, StemLoopTrunc_24 etc.) show a nearly 1500 increase in indel activity relative to the parent counterpart.

TABLE 3
Indel Activity Relative to Parent sgRNA

| sgRNA Reference | Result Summary |
|---|---|
| 5truncate_4 | Decreased indel activity |
| 5truncate_6 | Decreased indel activity |
| 5truncate_9 | Decreased indel activity |
| 5truncate_10 | Decreased indel activity |
| 5truncate_11 | Decreased indel activity |
| 5truncate_12 | Decreased indel activity |
| 5truncate_13 | Decreased indel activity |
| 5truncate_14 | Decreased indel activity |
| 5truncate_15 | Decreased indel activity |
| 5truncate_16 | Decreased indel activity |
| 5truncate_17 | Decreased indel activity |
| 5truncate_19 | Decreased indel activity |
| 5truncate_20 | Decreased indel activity |
| StemLoopTrunc_1 | Comparable to parent activity |
| StemLoopTrunc_2 | Increased indel activity |
| StemLoopTrunc_3 | Comparable to parent activity |
| StemLoopTrunc_4 | Comparable to parent activity |
| StemLoopTrunc_5 | Comparable to parent activity |
| StemLoopTrunc_8 | Comparable to parent activity |
| StemLoopTrunc_9 | Increased indel activity |
| StemLoopTrunc_10 | Comparable to parent activity |
| StemLoopTrunc_11 | Increased indel activity |
| StemLoopTrunc_13 | Increased indel activity |
| StemLoopTrunc_14 | Comparable to parent activity |
| StemLoopTrunc_15 | Increased indel activity |
| StemLoopTrunc_17 | Increased indel activity |
| StemLoopTrunc_18 | Comparable to parent activity |
| StemLoopTrunc_20 | Increased indel activity |
| StemLoopTrunc_21 | Comparable to parent activity |
| StemLoopTrunc_23 | Increased indel activity |
| StemLoopTrunc_24 | Increased indel activity |
| StemLoopTrunc_25 | Increased indel activity |
| StemLoopTrunc_26 | Increased indel activity |
| StemLoopTrunc_27 | Increased indel activity |
| StemLoopTrunc_28 | Comparable to parent activity |
| StemLoopTrunc_29 | Decreased indel activity |
| StemLoopTrunc_31 | Decreased indel activity |
| StemLoopTrunc_32 | Comparable to parent activity |
| StemLoopTrunc_33 | Comparable to parent activity |
| StemLoopTrunc_34 | Increased indel activity |
| StemLoopTrunc_35 | n/a |
| StemLoopTrunc_36 | Increased indel activity |
| StemPairing_3 | Increased indel activity |
| StemPairing_4 | Increased indel activity |
| StemPairing_5 | Increased indel activity |
| StemPairing_6 | Increased indel activity |
| StemPairing_7 | Increased indel activity |
| StemPairing_8 | Increased indel activity |
| StemPairing_9 | Increased indel activity |
| StemPairing_10 | Increased indel activity |
| StemPairing_11 | Increased indel activity |
| StemPairing_12 | Increased indel activity |
| StemPairing_13 | Increased indel activity |
| StemPairing_14 | Increased indel activity |
| StemPairing_15 | Increased indel activity |
| StemPairing_16 | Increased indel activity |
| StemPairing_17 | Increased indel activity |
| StemPairing_18 | Increased indel activity |
| StemPairing_19 | Increased indel activity |
| StemPairing_20 | Increased indel activity |
| 5elongate_1 | Increased indel activity |
| 5elongate_2 | Comparable to parent activity |
| 5elongate_3 | Increased indel activity |
| 5elongate_5 | Comparable to parent activity |
| 5elongate_6 | Comparable to parent activity |

### Example 2: RNA-Templated Editing in HEK293T Cells Using Engineered Editing Template RNAs and DNA Delivery of Type V Nuclease-RT Fusion Polypeptide

This Example describes the editing of the mammalian genome with a plasmid-encoded nuclease-MMLV fusion protein and editing template RNAs. A fusion of the nuclease (SEQ ID NO: 1) with mutant MMLV reverse transcriptase (RT) was cloned. The configuration of the C-terminal RT fusion to the nuclease, and the amino acid sequence of the fusion polypeptide are shown in Table 1 (SEQ ID NO: 5). A working solution of plasmid for expression of the nuclease-RT fusion was prepared in water.

Editing template RNAs of Table 4 were synthesized by IDT. A reverse transcription template (RTT) sequence and primer binding site (PBS) were fused to the 5′ end of an RNA guide. The editing template RNAs were designed to introduce 4-nucleotide substitutions occurring at positions 9-12 of the spacer sequence. The RTT of each is 45-nucleotides in length, and the PBS of each is 30-nucleotides in length. Several of the editing template RNAs were end protected with either xrRNA (tgtcaggcctgctagtcagccacagtttggggaaagctgtgcagcctgtaacccccccaggagaagctgggAAAAA) (SEQ ID NO: 72) or tRNA (CCAGTGGTCTAGTGGTAGAATAGTACCCTGCCACGGTACAGACCCGGGTTCGATTCCCGGCTGGAAATAAAAA) (SEQ ID NO: 73). For each cloning, oligos (two on the top strand and two on the bottom strand) were transferred into a single well. The oligo mixture was first treated and annealed with T4 PNK kinase, diluted, then followed by ligation into the plasmid backbone. After transformation into E. coli competent cells, individual colonies were picked, and Sanger Sequencing was used to confirm the insertion of the corresponding editing template sequence. Prior to transient transfection, a working solution of each editing template RNA was prepared in water (editing template RNA working solution).

TABLE 4
Sequences of Editing Template RNAs

| Editing | | | |
|---|---|---|---|
| Template | | | |
| RNA | Whole Sequence* | Target | Target sequence |
| sgRNA_C9 | CACCGCCGUCAUCAACAUUGCCUUUC | EMX1_TTTC | TGTTGCCCTCA |
| NoProtect_ | UGUUGCCCAGUAAACUUAUCAUACGG | | TAACTTATC |
| EMX1_TTTC | AGCCAGGCUAUAUAAUUAGCUACUUC | | (SEQ ID NO: 2) |
| | UCCGUGAGGUGCAGCCACGAGGUGC | | |
| | GAAUAGGAGUACGCAGCAAUGUGCU | | |
| | GAACGUUCGCACGAAACCUGGCAUU | | |
| | AUUGAAAACCAAUGCCAGGAACCGU | | |
| | CUCCAUAAAUAUGUGUGGUGACGGA | | |
| | CUGCAAGUGUUGCCCUCAUAACUUAU | | |
| | CUUUUUUGCG (SEQ ID NO: 75) | | |
| sgRNA_C9_ | CACCGUGAGGAUGUAGUCACGGAUUC | VEGFA_T8 | ATTATCAGCAA |
| NoProtect_ | AUUAUCAGGUUCUGGCUGCAGGGUGC | | GTGGCTGCA |
| VEGFA_T8 | CUGAUCUGUGCCAGGGUUAAGCAUGC | | (SEQ ID NO: 74) |
| | UGCGUGAGGUGCAGCCACGAGGUGC | | |
| | GAAUAGGAGUACGCAGCAAUGUGCU | | |
| | GAACGUUCGCACGAAACCUGGCAUU | | |
| | AUUGAAAACCAAUGCCAGGAACCGU | | |
| | CUCCAUAAAUAUGUGUGGUGACGGA | | |
| | CUGCAAGAUUAUCAGCAAGUGGCUG | | |
| | CAUUUUUUGCG (SEQ ID NO: 76) | | |
| sgRNA_C9_ | CACCGUGUCAGGCCUGCUAGUCAGCC | EMX1_TTTC | TGTTGCCCTCA |
| xrRNA_ | ACAGUUUGGGGAAAGCUGUGCAGCCU | | TAACTTATC |
| EMX1_TTTC | GUAACCCCCCCAGGAGAAGCUGGGAA | | (SEQ ID NO: 2) |
| | AAACCGUCAUCAACAUUGCCUUUCUG | | |
| | UUGCCCAGUAAACUUAUCAUACGGAG | | |
| | CCAGGCUAUAUAAUUAGCUACUUCUC | | |
| | CGUGAGGUGCAGCCACGAGGUGCG | | |
| | AAUAGGAGUACGCAGCAAUGUGCUG | | |
| | AACGUUCGCACGAAACCUGGCAUUA | | |
| | UUGAAAACCAAUGCCAGGAACCGUC | | |
| | UCCAUAAAUAUGUGUGGUGACGGAC | | |
| | UGCAAGUGUUGCCCUCAUAACUUAUC | | |
| | UUUUUUGCG (SEQ ID NO: 77) | | |
| sgRNA_C9_ | CACCGUGUCAGGCCUGCUAGUCAGCC | VEGFA_T8 | ATTATCAGCAA |
| xrRNA_ | ACAGUUUGGGGAAAGCUGUGCAGCCU | | GTGGCTGCA |
| VEGFA_T8 | GUAACCCCCCCAGGAGAAGCUGGGAA | | (SEQ ID NO: 74) |
| | AAAUGAGGAUGUAGUCACGGAUUCAU | | |
| | UAUCAGGUUCUGGCUGCAGGGUGCCU | | |
| | GAUCUGUGCCAGGGUUAAGCAUGCUG | | |
| | CGUGAGGUGCAGCCACGAGGUGCG | | |
| | AAUAGGAGUACGCAGCAAUGUGCUG | | |
| | AACGUUCGCACGAAACCUGGCAUUA | | |
| | UUGAAAACCAAUGCCAGGAACCGUC | | |
| | UCCAUAAAUAUGUGUGGUGACGGAC | | |
| | UGCAAGAUUAUCAGCAAGUGGCUGC | | |
| | AUUUUUUGCG (SEQ ID NO: 78) | | |
| sgRNA_C9_ | CACCGCCAGUGGUCUAGUGGUAGAAU | EMX1_TTTC | TGTTGCCCTCA |
| tRNA_EMX1_ | AGUACCCUGCCACGGUACAGACCCGG | | TAACTTATC |
| TTTC | GUUCGAUUCCCGGCUGGAAAUAAAAA | | (SEQ ID NO: 2) |
| | CCGUCAUCAACAUUGCCUUUCUGUUG | | |
| | CCCAGUAAACUUAUCAUACGGAGCCA | | |
| | GGCUAUAUAAUUAGCUACUUCUCCGU | | |
| | GAGGUGCAGCCACGAGGUGCGAAUA | | |
| | GGAGUACGCAGCAAUGUGCUGAACG | | |
| | UUCGCACGAAACCUGGCAUUAUUGA | | |
| | AAACCAAUGCCAGGAACCGUCUCCA | | |
| | UAAAUAUGUGUGGUGACGGACUGCA | | |
| | AGUGUUGCCCUCAUAACUUAUCUUUU | | |
| | UUGCG (SEQ ID NO: 79) | | |
| sgRNA_C9_ | CACCGCCAGUGGUCUAGUGGUAGAAU | VEGFA_T8 | ATTATCAGCAA |
| tRNA_ | AGUACCCUGCCACGGUACAGACCCGG | | GTGGCTGCA |
| VEGFA_T8 | GUUCGAUUCCCGGCUGGAAAUAAAAA | | (SEQ ID NO: 74) |
| | UGAGGAUGUAGUCACGGAUUCAUUAU | | |
| | CAGGUUCUGGCUGCAGGGUGCCUGAU | | |
| | CUGUGCCAGGGUUAAGCAUGCUGCGU | | |
| | GAGGUGCAGCCACGAGGUGCGAAUA | | |
| | GGAGUACGCAGCAAUGUGCUGAACG | | |
| | UUCGCACGAAACCUGGCAUUAUUGA | | |
| | AAACCAAUGCCAGGAACCGUCUCCA | | |
| | UAAAUAUGUGUGGUGACGGACUGCA | | |
| | AGAUUAUCAGCAAGUGGCUGCAUUU | | |
| | UUUGCG (SEQ ID NO: 80) | | |
| sgRNA_C10_ | CACCGCCGUCAUCAACAUUGCCUUUC | EMX1_TTTC | TGTTGCCCTCA |
| NoProtect_ | UGUUGCCCAGUAAACUUAUCAUACGG | | TAACTTATC |
| EMX1_TTTC | AGCCAGGCUAUAUAAUUAGCUACUUC | | (SEQ ID NO: 2) |
| | UCUUCGUGAGGUGCAGCCACGAGGU | | |
| | GCGAAUAGGAGUACGCAGCAAUGUG | | |
| | CUGAACGUUCGCACGAAACCUGGCA | | |
| | UUAUUGAAAACCAAUGCCAGGAACC | | |
| | GUCUCCAUAAAUAUGUGUGGUGACG | | |
| | GACUGCAAGUGUUGCCCUCAUAACU | | |
| | UAUCUUUUUUGCG (SEQ ID NO: 81) | | |
| sgRNA_C10_ | CACCGUGAGGAUGUAGUCACGGAUUC | VEGFA_T8 | ATTATCAGCAA |
| NoProtect_ | AUUAUCAGGUUCUGGCUGCAGGGUGC | | GTGGCTGCA |
| VEGFA_T8 | CUGAUCUGUGCCAGGGUUAAGCAUGC | | (SEQ ID NO: 74) |
| | UGUUCGUGAGGUGCAGCCACGAGGU | | |
| | GCGAAUAGGAGUACGCAGCAAUGUG | | |
| | CUGAACGUUCGCACGAAACCUGGCA | | |
| | UUAUUGAAAACCAAUGCCAGGAACC | | |
| | GUCUCCAUAAAUAUGUGUGGUGACG | | |
| | GACUGCAAGAUUAUCAGCAAGUGGC | | |
| | UGCAUUUUUUGCG (SEQ ID NO: 82) | | |
| sgRNA_C10_ | CACCGUGUCAGGCCUGCUAGUCAGCC | EMX1_TTTC | TGTTGCCCTCA |
| xrRNA_ | ACAGUUUGGGGAAAGCUGUGCAGCCU | | TAACTTATC |
| EMX1_TTTC | GUAACCCCCCCAGGAGAAGCUGGGAA | | (SEQ ID NO: 2) |
| | AAACCGUCAUCAACAUUGCCUUUCUG | | |
| | UUGCCCAGUAAACUUAUCAUACGGAG | | |
| | CCAGGCUAUAUAAUUAGCUACUUCUC | | |
| | UUCGUGAGGUGCAGCCACGAGGUGC | | |
| | GAAUAGGAGUACGCAGCAAUGUGCU | | |
| | GAACGUUCGCACGAAACCUGGCAUU | | |
| | AUUGAAAACCAAUGCCAGGAACCGU | | |
| | CUCCAUAAAUAUGUGUGGUGACGGA | | |
| | CUGCAAGUGUUGCCCUCAUAACUUAU | | |
| | CUUUUUUGCG (SEQ ID NO: 83) | | |
| sgRNA_C10_ | CACCGUGUCAGGCCUGCUAGUCAGCC | VEGFA_T8 | ATTATCAGCAA |
| xrRNA_ | ACAGUUUGGGGAAAGCUGUGCAGCCU | | GTGGCTGCA |
| VEGFA_T8 | GUAACCCCCCCAGGAGAAGCUGGGAA | | (SEQ ID NO: 74) |
| | AAAUGAGGAUGUAGUCACGGAUUCAU | | |
| | UAUCAGGUUCUGGCUGCAGGGUGCCU | | |
| | GAUCUGUGCCAGGGUUAAGCAUGCUG | | |
| | UUCGUGAGGUGCAGCCACGAGGUGC | | |
| | GAAUAGGAGUACGCAGCAAUGUGCU | | |
| | GAACGUUCGCACGAAACCUGGCAUU | | |
| | AUUGAAAACCAAUGCCAGGAACCGU | | |
| | CUCCAUAAAUAUGUGUGGUGACGGA | | |
| | CUGCAAGAUUAUCAGCAAGUGGCUG | | |
| | CAUUUUUUGCG (SEQ ID NO: 84) | | |
| sgRNA_C10_ | CACCGCCAGUGGUCUAGUGGUAGAAU | EMX1_TTTC | TGTTGCCCTCA |
| tRNA_ | AGUACCCUGCCACGGUACAGACCCGG | | TAACTTATC |
| EMX1_TTTC | GUUCGAUUCCCGGCUGGAAAUAAAAA | | (SEQ ID NO: 2) |
| | CCGUCAUCAACAUUGCCUUUCUGUUG | | |
| | CCCAGUAAACUUAUCAUACGGAGCCA | | |
| | GGCUAUAUAAUUAGCUACUUCUCUUC | | |
| | GUGAGGUGCAGCCACGAGGUGCGA | | |
| | AUAGGAGUACGCAGCAAUGUGCUGA | | |
| | ACGUUCGCACGAAACCUGGCAUUAU | | |
| | UGAAAACCAAUGCCAGGAACCGUCU | | |
| | CCAUAAAUAUGUGUGGUGACGGACU | | |
| | GCAAGUGUUGCCCUCAUAACUUAUCU | | |
| | UUUUUGCG (SEQ ID NO: 85) | | |
| sgRNA_C10_ | CACCGCCAGUGGUCUAGUGGUAGAAU | VEGFA_T8 | ATTATCAGCAA |
| tRNA_ | AGUACCCUGCCACGGUACAGACCCGG | | GTGGCTGCA |
| VEGFA_T8 | GUUCGAUUCCCGGCUGGAAAUAAAAA | | (SEQ ID NO: 74) |
| | UGAGGAUGUAGUCACGGAUUCAUUAU | | |
| | CAGGUUCUGGCUGCAGGGUGCCUGAU | | |
| | CUGUGCCAGGGUUAAGCAUGCUGUUC | | |
| | GUGAGGUGCAGCCACGAGGUGCGA | | |
| | AUAGGAGUACGCAGCAAUGUGCUGA | | |
| | ACGUUCGCACGAAACCUGGCAUUAU | | |
| | UGAAAACCAAUGCCAGGAACCGUCU | | |
| | CCAUAAAUAUGUGUGGUGACGGACU | | |
| | GCAAGAUUAUCAGCAAGUGGCUGCA | | |
| | UUUUUUGCG (SEQ ID NO: 86) | | |
| sgRNA_C11_ | CACCGCCGUCAUCAACAUUGCCUUUC | EMX1_TTTC | TGTTGCCCTCA |
| NoProtect_ | UGUUGCCCAGUAAACUUAUCAUACGG | | TAACTTATC |
| EMX1_TTTC | AGCCAGGCUAUAUAAUUAGCUACUUC | | (SEQ ID NO: 2) |
| | UCCGUGAGGUGCAGCCACGAGGUGC | | |
| | GAAAGUACGCAGCAAUGUGCUGAAU | | |
| | UCGCACGAAAAUUGGCAGAAAACCU | | |
| | GCCAAUAACCAUCUCCAUAAAUAUG | | |
| | UGUGGUGAUGGACUGCAAGUGUUGC | | |
| | CCUCAUAACUUAUCUUUUUUGCG | | |
| | (SEQ ID NO: 87) | | |
| sgRNA_C11_ | CACCGUGAGGAUGUAGUCACGGAUUC | VEGFA_T8 | ATTATCAGCAA |
| NoProtect_ | AUUAUCAGGUUCUGGCUGCAGGGUGC | | GTGGCTGCA |
| VEGFA_T8 | CUGAUCUGUGCCAGGGUUAAGCAUGC | | (SEQ ID NO: 74) |
| | UGCGUGAGGUGCAGCCACGAGGUGC | | |
| | GAAAGUACGCAGCAAUGUGCUGAAU | | |
| | UCGCACGAAAAUUGGCAGAAAACCU | | |
| | GCCAAUAACCAUCUCCAUAAAUAUG | | |
| | UGUGGUGAUGGACUGCAAGAUUAUC | | |
| | AGCAAGUGGCUGCAUUUUUUGCG | | |
| | (SEQ ID NO: 88) | | |
| sgRNA_C11_ | CACCGUGUCAGGCCUGCUAGUCAGCC | EMX1_TTTC | TGTTGCCCTCA |
| xrRNA_ | ACAGUUUGGGGAAAGCUGUGCAGCCU | | TAACTTATC |
| EMX1_TTTC | GUAACCCCCCCAGGAGAAGCUGGGAA | | (SEQ ID NO: 2) |
| | AAACCGUCAUCAACAUUGCCUUUCUG | | |
| | UUGCCCAGUAAACUUAUCAUACGGAG | | |
| | CCAGGCUAUAUAAUUAGCUACUUCUC | | |
| | CGUGAGGUGCAGCCACGAGGUGCG | | |
| | AAAGUACGCAGCAAUGUGCUGAAUU | | |
| | CGCACGAAAAUUGGCAGAAAACCUG | | |
| | CCAAUAACCAUCUCCAUAAAUAUGU | | |
| | GUGGUGAUGGACUGCAAGUGUUGCC | | |
| | CUCAUAACUUAUCUUUUUUGCG (SEQ | | |
| | ID NO: 89) | | |
| sgRNA_C11_ | CACCGUGUCAGGCCUGCUAGUCAGCC | VEGFA_T8 | ATTATCAGCAA |
| xrRNA_ | ACAGUUUGGGGAAAGCUGUGCAGCCU | | GTGGCTGCA |
| VEGFA_T8 | GUAACCCCCCCAGGAGAAGCUGGGAA | | (SEQ ID NO: 74) |
| | AAAUGAGGAUGUAGUCACGGAUUCAU | | |
| | UAUCAGGUUCUGGCUGCAGGGUGCCU | | |
| | GAUCUGUGCCAGGGUUAAGCAUGCUG | | |
| | CGUGAGGUGCAGCCACGAGGUGCG | | |
| | AAAGUACGCAGCAAUGUGCUGAAUU | | |
| | CGCACGAAAAUUGGCAGAAAACCUG | | |
| | CCAAUAACCAUCUCCAUAAAUAUGU | | |
| | GUGGUGAUGGACUGCAAGAUUAUCA | | |
| | GCAAGUGGCUGCAUUUUUUGCG (SEQ | | |
| | ID NO: 90) | | |
| sgRNA_C11_ | CACCGCCAGUGGUCUAGUGGUAGAAU | EMX1_TTTC | TGTTGCCCTCA |
| tRNA_ | AGUACCCUGCCACGGUACAGACCCGG | | TAACTTATC |
| EMX1_TTTC | GUUCGAUUCCCGGCUGGAAAUAAAAA | | (SEQ ID NO: 2) |
| | CCGUCAUCAACAUUGCCUUUCUGUUG | | |
| | CCCAGUAAACUUAUCAUACGGAGCCA | | |
| | GGCUAUAUAAUUAGCUACUUCUCCGU | | |
| | GAGGUGCAGCCACGAGGUGCGAAA | | |
| | GUACGCAGCAAUGUGCUGAAUUCGC | | |
| | ACGAAAAUUGGCAGAAAACCUGCCA | | |
| | AUAACCAUCUCCAUAAAUAUGUGUG | | |
| | GUGAUGGACUGCAAGUGUUGCCCUC | | |
| | AUAACUUAUCUUUUUUGCG (SEQ ID | | |
| | NO: 91) | | |
| sgRNA_C11_ | CACCGCCAGUGGUCUAGUGGUAGAAU | VEGFA_T8 | ATTATCAGCAA |
| tRNA_ | AGUACCCUGCCACGGUACAGACCCGG | | GTGGCTGCA |
| VEGFA_T8 | GUUCGAUUCCCGGCUGGAAAUAAAAA | | (SEQ ID NO: 74) |
| | UGAGGAUGUAGUCACGGAUUCAUUAU | | |
| | CAGGUUCUGGCUGCAGGGUGCCUGAU | | |
| | CUGUGCCAGGGUUAAGCAUGCUGCGU | | |
| | GAGGUGCAGCCACGAGGUGCGAAA | | |
| | GUACGCAGCAAUGUGCUGAAUUCGC | | |
| | ACGAAAAUUGGCAGAAAACCUGCCA | | |
| | AUAACCAUCUCCAUAAAUAUGUGUG | | |
| | GUGAUGGACUGCAAGAUUAUCAGCA | | |
| | AGUGGCUGCAUUUUUUGCG (SEQ ID | | |
| | NO: 92) | | |
| sgRNA_C12_ | CACCGCCGUCAUCAACAUUGCCUUUC | EMX1_TTTC | TGTTGCCCTCA |
| NoProtect_ | UGUUGCCCAGUAAACUUAUCAUACGG | | TAACTTATC |
| EMX1_TTTC | AGCCAGGCUAUAUAAUUAGCUACUUC | | (SEQ ID NO: 2) |
| | UCGUGAGGUGCAGCCACGAGGUGCG | | |
| | AAAGUACGCCGCAAUGUGCGGAAUU | | |
| | CGCACGAAAGUUGGCGGAUUUCUUC | | |
| | GGAAAUCGAGCCAAUAACCAUCUCC | | |
| | AUAAAUAUGUGUGGUGAUGGACUGC | | |
| | AAGUGUUGCCCUCAUAACUUAUCUUU | | |
| | UUUGCG (SEQ ID NO: 93) | | |
| sgRNA_C12_ | CACCGUGAGGAUGUAGUCACGGAUUC | VEGFA_T8 | ATTATCAGCAA |
| NoProtect_ | AUUAUCAGGUUCUGGCUGCAGGGUGC | | GTGGCTGCA |
| VEGFA_T8 | CUGAUCUGUGCCAGGGUUAAGCAUGC | | (SEQ ID NO: 74) |
| | UGGUGAGGUGCAGCCACGAGGUGC | | |
| | GAAAGUACGCCGCAAUGUGCGGAAU | | |
| | UCGCACGAAAGUUGGCGGAUUUCUU | | |
| | CGGAAAUCGAGCCAAUAACCAUCUC | | |
| | CAUAAAUAUGUGUGGUGAUGGACUG | | |
| | CAAGAUUAUCAGCAAGUGGCUGCAUU | | |
| | UUUUGCG (SEQ ID NO: 94) | | |
| sgRNA_C12_ | CACCGUGUCAGGCCUGCUAGUCAGCC | EMX1_TTTC | TGTTGCCCTCA |
| xrRNA_ | ACAGUUUGGGGAAAGCUGUGCAGCCU | | TAACTTATC |
| EMX1_TTTC | GUAACCCCCCCAGGAGAAGCUGGGAA | | (SEQ ID NO: 2) |
| | AAACCGUCAUCAACAUUGCCUUUCUG | | |
| | UUGCCCAGUAAACUUAUCAUACGGAG | | |
| | CCAGGCUAUAUAAUUAGCUACUUCUC | | |
| | GUGAGGUGCAGCCACGAGGUGCGA | | |
| | AAGUACGCCGCAAUGUGCGGAAUUC | | |
| | GCACGAAAGUUGGCGGAUUUCUUCG | | |
| | GAAAUCGAGCCAAUAACCAUCUCCA | | |
| | UAAAUAUGUGUGGUGAUGGACUGCA | | |
| | AGUGUUGCCCUCAUAACUUAUCUUUU | | |
| | UUGCG (SEQ ID NO: 95) | | |
| sgRNA_C12_ | CACCGUGUCAGGCCUGCUAGUCAGCC | VEGFA_T8 | ATTATCAGCAA |
| xrRNA_ | ACAGUUUGGGGAAAGCUGUGCAGCCU | | GTGGCTGCA |
| VEGFA_T8 | GUAACCCCCCCAGGAGAAGCUGGGAA | | (SEQ ID NO: 74) |
| | AAAUGAGGAUGUAGUCACGGAUUCAU | | |
| | UAUCAGGUUCUGGCUGCAGGGUGCCU | | |
| | GAUCUGUGCCAGGGUUAAGCAUGCUG | | |
| | GUGAGGUGCAGCCACGAGGUGCGA | | |
| | AAGUACGCCGCAAUGUGCGGAAUUC | | |
| | GCACGAAAGUUGGCGGAUUUCUUCG | | |
| | GAAAUCGAGCCAAUAACCAUCUCCA | | |
| | UAAAUAUGUGUGGUGAUGGACUGCA | | |
| | AGAUUAUCAGCAAGUGGCUGCAUUUU | | |
| | UUGCG (SEQ ID NO: 96) | | |
| sgRNA_C12_ | CACCGCCAGUGGUCUAGUGGUAGAAU | EMX1_TTTC | TGTTGCCCTCA |
| tRNA_ | AGUACCCUGCCACGGUACAGACCCGG | | TAACTTATC |
| EMX1_TTTC | GUUCGAUUCCCGGCUGGAAAUAAAAA | | (SEQ ID NO: 2) |
| | CCGUCAUCAACAUUGCCUUUCUGUUG | | |
| | CCCAGUAAACUUAUCAUACGGAGCCA | | |
| | GGCUAUAUAAUUAGCUACUUCUCGUG | | |
| | AGGUGCAGCCACGAGGUGCGAAAGU | | |
| | ACGCCGCAAUGUGCGGAAUUCGCAC | | |
| | GAAAGUUGGCGGAUUUCUUCGGAAA | | |
| | UCGAGCCAAUAACCAUCUCCAUAAA | | |
| | UAUGUGUGGUGAUGGACUGCAAGUG | | |
| | UUGCCCUCAUAACUUAUCUUUUUUGC | | |
| | G (SEQ ID NO: 97) | | |
| sgRNA_C12_ | CACCGCCAGUGGUCUAGUGGUAGAAU | VEGFA_T8 | ATTATCAGCAA |
| tRNA_ | AGUACCCUGCCACGGUACAGACCCGG | | GTGGCTGCA |
| VEGFA_T8 | GUUCGAUUCCCGGCUGGAAAUAAAAA | | (SEQ ID NO: 74) |
| | UGAGGAUGUAGUCACGGAUUCAUUAU | | |
| | CAGGUUCUGGCUGCAGGGUGCCUGAU | | |
| | CUGUGCCAGGGUUAAGCAUGCUGGUG | | |
| | AGGUGCAGCCACGAGGUGCGAAAGU | | |
| | ACGCCGCAAUGUGCGGAAUUCGCAC | | |
| | GAAAGUUGGCGGAUUUCUUCGGAAA | | |
| | UCGAGCCAAUAACCAUCUCCAUAAA | | |
| | UAUGUGUGGUGAUGGACUGCAAGAU | | |
| | UAUCAGCAAGUGGCUGCAUUUUUUGC | | |
| | G (SEQ ID NO: 98) | | |
| sgRNA_C13_ | CACCGCCGUCAUCAACAUUGCCUUUC | EMX1_TTTC | TGTTGCCCTCA |
| NoProtect_ | UGUUGCCCAGUAAACUUAUCAUACGG | | TAACTTATC |
| EMX1_TTTC | AGCCAGGCUAUAUAAUUAGCUACUUC | | (SEQ ID NO: 2) |
| | UCCGUGAGGUGCAGCCACGAGGUGC | | |
| | GAAAGUACGCCGCAAUGUGCGGAAU | | |
| | UCGCACGAAACUUGGCAUUAUUGAA | | |
| | AACCAAUGCCAAGAAUCCUCUCCAA | | |
| | AAUAUGUUGGUGAGGAACUGCAAGU | | |
| | GUUGCCCUCAUAACUUAUCUUUUUUG | | |
| | CG (SEQ ID NO: 99) | | |
| sgRNA_C13_ | CACCGUGAGGAUGUAGUCACGGAUUC | VEGFA_T8 | ATTATCAGCAA |
| NoProtect_ | AUUAUCAGGUUCUGGCUGCAGGGUGC | | GTGGCTGCA |
| VEGFA_T8 | CUGAUCUGUGCCAGGGUUAAGCAUGC | | (SEQ ID NO: 74) |
| | UGCGUGAGGUGCAGCCACGAGGUGC | | |
| | GAAAGUACGCCGCAAUGUGCGGAAU | | |
| | UCGCACGAAACUUGGCAUUAUUGAA | | |
| | AACCAAUGCCAAGAAUCCUCUCCAA | | |
| | AAUAUGUUGGUGAGGAACUGCAAGA | | |
| | UUAUCAGCAAGUGGCUGCAUUUUUUG | | |
| | CG (SEQ ID NO: 100) | | |
| sgRNA_C13_ | CACCGUGUCAGGCCUGCUAGUCAGCC | EMX1_TTTC | TGTTGCCCTCA |
| xrRNA_ | ACAGUUUGGGGAAAGCUGUGCAGCCU | | TAACTTATC |
| EMX1_TTTC | GUAACCCCCCCAGGAGAAGCUGGGAA | | (SEQ ID NO: 2) |
| | AAACCGUCAUCAACAUUGCCUUUCUG | | |
| | UUGCCCAGUAAACUUAUCAUACGGAG | | |
| | CCAGGCUAUAUAAUUAGCUACUUCUC | | |
| | CGUGAGGUGCAGCCACGAGGUGCG | | |
| | AAAGUACGCCGCAAUGUGCGGAAUU | | |
| | CGCACGAAACUUGGCAUUAUUGAAA | | |
| | ACCAAUGCCAAGAAUCCUCUCCAAA | | |
| | AUAUGUUGGUGAGGAACUGCAAGUG | | |
| | UUGCCCUCAUAACUUAUCUUUUUUGC | | |
| | G (SEQ ID NO: 101) | | |
| sgRNA_C13_ | CACCGUGUCAGGCCUGCUAGUCAGCC | VEGFA_T8 | ATTATCAGCAA |
| xrRNA_ | ACAGUUUGGGGAAAGCUGUGCAGCCU | | GTGGCTGCA |
| VEGFA_T8 | GUAACCCCCCCAGGAGAAGCUGGGAA | | (SEQ ID NO: 74) |
| | AAAUGAGGAUGUAGUCACGGAUUCAU | | |
| | UAUCAGGUUCUGGCUGCAGGGUGCCU | | |
| | GAUCUGUGCCAGGGUUAAGCAUGCUG | | |
| | CGUGAGGUGCAGCCACGAGGUGCG | | |
| | AAAGUACGCCGCAAUGUGCGGAAUU | | |
| | CGCACGAAACUUGGCAUUAUUGAAA | | |
| | ACCAAUGCCAAGAAUCCUCUCCAAA | | |
| | AUAUGUUGGUGAGGAACUGCAAGAU | | |
| | UAUCAGCAAGUGGCUGCAUUUUUUGC | | |
| | G (SEQ ID NO: 102) | | |
| sgRNA_C13_ | CACCGCCAGUGGUCUAGUGGUAGAAU | EMX1_TTTC | TGTTGCCCTCA |
| tRNA_ | AGUACCCUGCCACGGUACAGACCCGG | | TAACTTATC |
| EMX1_TTTC | GUUCGAUUCCCGGCUGGAAAUAAAAA | | (SEQ ID NO: 2) |
| | CCGUCAUCAACAUUGCCUUUCUGUUG | | |
| | CCCAGUAAACUUAUCAUACGGAGCCA | | |
| | GGCUAUAUAAUUAGCUACUUCUCCGU | | |
| | GAGGUGCAGCCACGAGGUGCGAAA | | |
| | GUACGCCGCAAUGUGCGGAAUUCGC | | |
| | ACGAAACUUGGCAUUAUUGAAAACC | | |
| | AAUGCCAAGAAUCCUCUCCAAAAUA | | |
| | UGUUGGUGAGGAACUGCAAGUGUUG | | |
| | CCCUCAUAACUUAUCUUUUUUGCG | | |
| | (SEQ ID NO: 103) | | |
| sgRNA_C13_ | CACCGCCAGUGGUCUAGUGGUAGAAU | VEGFA_T8 | ATTATCAGCAA |
| tRNA_ | AGUACCCUGCCACGGUACAGACCCGG | | GTGGCTGCA |
| VEGFA_T8 | GUUCGAUUCCCGGCUGGAAAUAAAAA | | (SEQ ID NO: 74) |
| | UGAGGAUGUAGUCACGGAUUCAUUAU | | |
| | CAGGUUCUGGCUGCAGGGUGCCUGAU | | |
| | CUGUGCCAGGGUUAAGCAUGCUGCGU | | |
| | GAGGUGCAGCCACGAGGUGCGAAA | | |
| | GUACGCCGCAAUGUGCGGAAUUCGC | | |
| | ACGAAACUUGGCAUUAUUGAAAACC | | |
| | AAUGCCAAGAAUCCUCUCCAAAAUA | | |
| | UGUUGGUGAGGAACUGCAAGAUUAU | | |
| | CAGCAAGUGGCUGCAUUUUUUGCG | | |
| | (SEQ ID NO: 104) | | |
| sgRNA_C14_ | CACCGCCGUCAUCAACAUUGCCUUUC | EMX1_TTTC | TGTTGCCCTCA |
| NoProtect_ | UGUUGCCCAGUAAACUUAUCAUACGG | | TAACTTATC |
| EMX1_TTTC | AGCCAGGCUAUAUAAUUAGCUACUUC | | (SEQ ID NO: 2) |
| | UCGUGAGGUGCAGCCACGAGGUGCG | | |
| | AAAGUACGCCGCAAUGUGCGGAAUU | | |
| | CGCACGAAACUUGGCAUUAUUGAAA | | |
| | ACCAAUGCCAAGAAGCAUAUCCAUA | | |
| | UUAUCUGCGGAUGGAUGUGCACUGC | | |
| | AAGUGUUGCCCUCAUAACUUAUCUUU | | |
| | UUUGCG (SEQ ID NO: 105) | | |
| sgRNA_C14_ | CACCGUGAGGAUGUAGUCACGGAUUC | VEGFA_T8 | ATTATCAGCAA |
| NoProtect_ | AUUAUCAGGUUCUGGCUGCAGGGUGC | | GTGGCTGCA |
| VEGFA_T8 | CUGAUCUGUGCCAGGGUUAAGCAUGC | | (SEQ ID NO: 74) |
| | UGGUGAGGUGCAGCCACGAGGUGC | | |
| | GAAAGUACGCCGCAAUGUGCGGAAU | | |
| | UCGCACGAAACUUGGCAUUAUUGAA | | |
| | AACCAAUGCCAAGAAGCAUAUCCAU | | |
| | AUUAUCUGCGGAUGGAUGUGCACUG | | |
| | CAAGAUUAUCAGCAAGUGGCUGCAUU | | |
| | UUUUGCG (SEQ ID NO: 106) | | |
| sgRNA_C14_ | CACCGUGUCAGGCCUGCUAGUCAGCC | EMX1_TTTC | TGTTGCCCTCA |
| xrRNA_ | ACAGUUUGGGGAAAGCUGUGCAGCCU | | TAACTTATC |
| EMX1_TTTC | GUAACCCCCCCAGGAGAAGCUGGGAA | | (SEQ ID NO: 2) |
| | AAACCGUCAUCAACAUUGCCUUUCUG | | |
| | UUGCCCAGUAAACUUAUCAUACGGAG | | |
| | CCAGGCUAUAUAAUUAGCUACUUCUC | | |
| | GUGAGGUGCAGCCACGAGGUGCGA | | |
| | AAGUACGCCGCAAUGUGCGGAAUUC | | |
| | GCACGAAACUUGGCAUUAUUGAAAA | | |
| | CCAAUGCCAAGAAGCAUAUCCAUAU | | |
| | UAUCUGCGGAUGGAUGUGCACUGCA | | |
| | AGUGUUGCCCUCAUAACUUAUCUUUU | | |
| | UUGCG (SEQ ID NO: 107) | | |
| sgRNA_C14_ | CACCGUGUCAGGCCUGCUAGUCAGCC | VEGFA_T8 | ATTATCAGCAA |
| xrRNA_ | ACAGUUUGGGGAAAGCUGUGCAGCCU | | GTGGCTGCA |
| VEGFA_T8 | GUAACCCCCCCAGGAGAAGCUGGGAA | | (SEQ ID NO: 74) |
| | AAAUGAGGAUGUAGUCACGGAUUCAU | | |
| | UAUCAGGUUCUGGCUGCAGGGUGCCU | | |
| | GAUCUGUGCCAGGGUUAAGCAUGCUG | | |
| | GUGAGGUGCAGCCACGAGGUGCGA | | |
| | AAGUACGCCGCAAUGUGCGGAAUUC | | |
| | GCACGAAACUUGGCAUUAUUGAAAA | | |
| | CCAAUGCCAAGAAGCAUAUCCAUAU | | |
| | UAUCUGCGGAUGGAUGUGCACUGCA | | |
| | AGAUUAUCAGCAAGUGGCUGCAUUUU | | |
| | UUGCG (SEQ ID NO: 108) | | |
| sgRNA_C14_ | CACCGCCAGUGGUCUAGUGGUAGAAU | EMX1_TTTC | TGTTGCCCTCA |
| tRNA_ | AGUACCCUGCCACGGUACAGACCCGG | | TAACTTATC |
| EMX1_TTTC | GUUCGAUUCCCGGCUGGAAAUAAAAA | | (SEQ ID NO: 2) |
| | CCGUCAUCAACAUUGCCUUUCUGUUG | | |
| | CCCAGUAAACUUAUCAUACGGAGCCA | | |
| | GGCUAUAUAAUUAGCUACUUCUCGUG | | |
| | AGGUGCAGCCACGAGGUGCGAAAGU | | |
| | ACGCCGCAAUGUGCGGAAUUCGCAC | | |
| | GAAACUUGGCAUUAUUGAAAACCAA | | |
| | UGCCAAGAAGCAUAUCCAUAUUAUC | | |
| | UGCGGAUGGAUGUGCACUGCAAGUG | | |
| | UUGCCCUCAUAACUUAUCUUUUUUGC | | |
| | G (SEQ ID NO: 109) | | |
| sgRNA_C14_ | CACCGCCAGUGGUCUAGUGGUAGAAU | VEGFA_T8 | ATTATCAGCAA |
| tRNA_ | AGUACCCUGCCACGGUACAGACCCGG | | GTGGCTGCA |
| VEGFA_T8 | GUUCGAUUCCCGGCUGGAAAUAAAAA | | (SEQ ID NO: 74) |
| | UGAGGAUGUAGUCACGGAUUCAUUAU | | |
| | CAGGUUCUGGCUGCAGGGUGCCUGAU | | |
| | CUGUGCCAGGGUUAAGCAUGCUGGUG | | |
| | AGGUGCAGCCACGAGGUGCGAAAGU | | |
| | ACGCCGCAAUGUGCGGAAUUCGCAC | | |
| | GAAACUUGGCAUUAUUGAAAACCAA | | |
| | UGCCAAGAAGCAUAUCCAUAUUAUC | | |
| | UGCGGAUGGAUGUGCACUGCAAGAU | | |
| | UAUCAGCAAGUGGCUGCAUUUUUUGC | | |
| | G (SEQ ID NO: 110) | | |
| sgRNA_C15_ | CACCGCCGUCAUCAACAUUGCCUUUC | EMX1_TTTC | TGTTGCCCTCA |
| NoProtect_ | UGUUGCCCAGUAAACUUAUCAUACGG | | TAACTTATC |
| EMX1_TTTC | AGCCAGGCUAUAUAAUUAGCUACUUC | | (SEQ ID NO: 2) |
| | UCCGUGAGGUGCAGCCACGAGGUGC | | |
| | GAAUAGGAGUACGCCGCAAUGUGCG | | |
| | GAACGUUCGCACGAAACUUGGCAGA | | |
| | AAACCUGCCAAGAAUCCUAUCCAAA | | |
| | AUAUGUUGGUUAGGAACUGCAAGUG | | |
| | UUGCCCUCAUAACUUAUCUUUUUUGC | | |
| | G (SEQ ID NO: 111) | | |
| sgRNA_C15_ | CACCGUGAGGAUGUAGUCACGGAUUC | VEGFA_T8 | ATTATCAGCAA |
| NoProtect_ | AUUAUCAGGUUCUGGCUGCAGGGUGC | | GTGGCTGCA |
| VEGFA_T8 | CUGAUCUGUGCCAGGGUUAAGCAUGC | | (SEQ ID NO: 74) |
| | UGCGUGAGGUGCAGCCACGAGGUGC | | |
| | GAAUAGGAGUACGCCGCAAUGUGCG | | |
| | GAACGUUCGCACGAAACUUGGCAGA | | |
| | AAACCUGCCAAGAAUCCUAUCCAAA | | |
| | AUAUGUUGGUUAGGAACUGCAAGAU | | |
| | UAUCAGCAAGUGGCUGCAUUUUUUGC | | |
| | G (SEQ ID NO: 112) | | |
| sgRNA_C15_ | CACCGUGUCAGGCCUGCUAGUCAGCC | EMX1_TTTC | TGTTGCCCTCA |
| xrRNA_ | ACAGUUUGGGGAAAGCUGUGCAGCCU | | TAACTTATC |
| EMX1_TTTC | GUAACCCCCCCAGGAGAAGCUGGGAA | | (SEQ ID NO: 2) |
| | AAACCGUCAUCAACAUUGCCUUUCUG | | |
| | UUGCCCAGUAAACUUAUCAUACGGAG | | |
| | CCAGGCUAUAUAAUUAGCUACUUCUC | | |
| | CGUGAGGUGCAGCCACGAGGUGCG | | |
| | AAUAGGAGUACGCCGCAAUGUGCGG | | |
| | AACGUUCGCACGAAACUUGGCAGAA | | |
| | AACCUGCCAAGAAUCCUAUCCAAAA | | |
| | UAUGUUGGUUAGGAACUGCAAGUGU | | |
| | UGCCCUCAUAACUUAUCUUUUUUGCG | | |
| | (SEQ ID NO: 113) | | |
| sgRNA_C15_ | CACCGUGUCAGGCCUGCUAGUCAGCC | VEGFA_T8 | ATTATCAGCAA |
| xrRNA_ | ACAGUUUGGGGAAAGCUGUGCAGCCU | | GTGGCTGCA |
| VEGFA_T8 | GUAACCCCCCCAGGAGAAGCUGGGAA | | (SEQ ID NO: 74) |
| | AAAUGAGGAUGUAGUCACGGAUUCAU | | |
| | UAUCAGGUUCUGGCUGCAGGGUGCCU | | |
| | GAUCUGUGCCAGGGUUAAGCAUGCUG | | |
| | CGUGAGGUGCAGCCACGAGGUGCG | | |
| | AAUAGGAGUACGCCGCAAUGUGCGG | | |
| | AACGUUCGCACGAAACUUGGCAGAA | | |
| | AACCUGCCAAGAAUCCUAUCCAAAA | | |
| | UAUGUUGGUUAGGAACUGCAAGAUU | | |
| | AUCAGCAAGUGGCUGCAUUUUUUGCG | | |
| | (SEQ ID NO: 114) | | |
| sgRNA_C15_ | CACCGCCAGUGGUCUAGUGGUAGAAU | EMX1_TTTC | TGTTGCCCTCA |
| tRNA_ | AGUACCCUGCCACGGUACAGACCCGG | | TAACTTATC |
| EMX1_TTTC | GUUCGAUUCCCGGCUGGAAAUAAAAA | | (SEQ ID NO: 2) |
| | CCGUCAUCAACAUUGCCUUUCUGUUG | | |
| | CCCAGUAAACUUAUCAUACGGAGCCA | | |
| | GGCUAUAUAAUUAGCUACUUCUCCGU | | |
| | GAGGUGCAGCCACGAGGUGCGAAUA | | |
| | GGAGUACGCCGCAAUGUGCGGAACG | | |
| | UUCGCACGAAACUUGGCAGAAAACC | | |
| | UGCCAAGAAUCCUAUCCAAAAUAUG | | |
| | UUGGUUAGGAACUGCAAGUGUUGCC | | |
| | CUCAUAACUUAUCUUUUUUGCG (SEQ | | |
| | ID NO: 115) | | |
| sgRNA_C15_ | CACCGCCAGUGGUCUAGUGGUAGAAU | VEGFA_T8 | ATTATCAGCAA |
| tRNA_ | AGUACCCUGCCACGGUACAGACCCGG | | GTGGCTGCA |
| VEGFA_T8 | GUUCGAUUCCCGGCUGGAAAUAAAAA | | (SEQ ID NO: 74) |
| | UGAGGAUGUAGUCACGGAUUCAUUAU | | |
| | CAGGUUCUGGCUGCAGGGUGCCUGAU | | |
| | CUGUGCCAGGGUUAAGCAUGCUGCGU | | |
| | GAGGUGCAGCCACGAGGUGCGAAUA | | |
| | GGAGUACGCCGCAAUGUGCGGAACG | | |
| | UUCGCACGAAACUUGGCAGAAAACC | | |
| | UGCCAAGAAUCCUAUCCAAAAUAUG | | |
| | UUGGUUAGGAACUGCAAGAUUAUCA | | |
| | GCAAGUGGCUGCAUUUUUUGCG (SEQ | | |
| | ID NO: 116) | | |
| sgRNA_C16_ | CACCGCCGUCAUCAACAUUGCCUUUC | EMX1_TTTC | TGTTGCCCTCA |
| NoProtect_ | UGUUGCCCAGUAAACUUAUCAUACGG | | TAACTTATC |
| EMX1_TTTC | AGCCAGGCUAUAUAAUUAGCUACUUC | | (SEQ ID NO: 2) |
| | UCCGUGAGGUGCAGCCACGAGGUGC | | |
| | GAAAGUACGCCGCAAUGUGCGGAAU | | |
| | UCGCACGAAAGUUGGCGGAUUUCUU | | |
| | CGGAAAUCGAGCCAAUAAGCAUAUC | | |
| | CAUAUUAUCUGCGGAUGGAUGUGCA | | |
| | CUGCAAGUGUUGCCCUCAUAACUUAU | | |
| | CUUUUUUGCG (SEQ ID NO: 117) | | |
| sgRNA_C16_ | CACCGUGAGGAUGUAGUCACGGAUUC | VEGFA_T8 | ATTATCAGCAA |
| NoProtect_ | AUUAUCAGGUUCUGGCUGCAGGGUGC | | GTGGCTGCA |
| VEGFA_T8 | CUGAUCUGUGCCAGGGUUAAGCAUGC | | (SEQ ID NO: 74) |
| | UGCGUGAGGUGCAGCCACGAGGUGC | | |
| | GAAAGUACGCCGCAAUGUGCGGAAU | | |
| | UCGCACGAAAGUUGGCGGAUUUCUU | | |
| | CGGAAAUCGAGCCAAUAAGCAUAUC | | |
| | CAUAUUAUCUGCGGAUGGAUGUGCA | | |
| | CUGCAAGAUUAUCAGCAAGUGGCUG | | |
| | CAUUUUUUGCG (SEQ ID NO: 118) | | |
| sgRNA_C16_ | CACCGUGUCAGGCCUGCUAGUCAGCC | EMX1_TTTC | TGTTGCCCTCA |
| xrRNA_ | ACAGUUUGGGGAAAGCUGUGCAGCCU | | TAACTTATC |
| EMX1_TTTC | GUAACCCCCCCAGGAGAAGCUGGGAA | | (SEQ ID NO: 2) |
| | AAACCGUCAUCAACAUUGCCUUUCUG | | |
| | UUGCCCAGUAAACUUAUCAUACGGAG | | |
| | CCAGGCUAUAUAAUUAGCUACUUCUC | | |
| | CGUGAGGUGCAGCCACGAGGUGCG | | |
| | AAAGUACGCCGCAAUGUGCGGAAUU | | |
| | CGCACGAAAGUUGGCGGAUUUCUUC | | |
| | GGAAAUCGAGCCAAUAAGCAUAUCC | | |
| | AUAUUAUCUGCGGAUGGAUGUGCAC | | |
| | UGCAAGUGUUGCCCUCAUAACUUAUC | | |
| | UUUUUUGCG (SEQ ID NO: 119) | | |
| sgRNA_C16_ | CACCGUGUCAGGCCUGCUAGUCAGCC | VEGFA_T8 | ATTATCAGCAA |
| xrRNA_ | ACAGUUUGGGGAAAGCUGUGCAGCCU | | GTGGCTGCA |
| VEGFA_T8 | GUAACCCCCCCAGGAGAAGCUGGGAA | | (SEQ ID NO: 74) |
| | AAAUGAGGAUGUAGUCACGGAUUCAU | | |
| | UAUCAGGUUCUGGCUGCAGGGUGCCU | | |
| | GAUCUGUGCCAGGGUUAAGCAUGCUG | | |
| | CGUGAGGUGCAGCCACGAGGUGCG | | |
| | AAAGUACGCCGCAAUGUGCGGAAUU | | |
| | CGCACGAAAGUUGGCGGAUUUCUUC | | |
| | GGAAAUCGAGCCAAUAAGCAUAUCC | | |
| | AUAUUAUCUGCGGAUGGAUGUGCAC | | |
| | UGCAAGAUUAUCAGCAAGUGGCUGC | | |
| | AUUUUUUGCG (SEQ ID NO: 120) | | |
| sgRNA_C16_ | CACCGCCAGUGGUCUAGUGGUAGAAU | EMX1_TTTC | TGTTGCCCTCA |
| tRNA_ | AGUACCCUGCCACGGUACAGACCCGG | | TAACTTATC |
| EMX1_TTTC | GUUCGAUUCCCGGCUGGAAAUAAAAA | | (SEQ ID NO: 2) |
| | CCGUCAUCAACAUUGCCUUUCUGUUG | | |
| | CCCAGUAAACUUAUCAUACGGAGCCA | | |
| | GGCUAUAUAAUUAGCUACUUCUCCGU | | |
| | GAGGUGCAGCCACGAGGUGCGAAA | | |
| | GUACGCCGCAAUGUGCGGAAUUCGC | | |
| | ACGAAAGUUGGCGGAUUUCUUCGGA | | |
| | AAUCGAGCCAAUAAGCAUAUCCAUA | | |
| | UUAUCUGCGGAUGGAUGUGCACUGC | | |
| | AAGUGUUGCCCUCAUAACUUAUCUUU | | |
| | UUUGCG (SEQ ID NO: 121) | | |
| sgRNA_C16_ | CACCGCCAGUGGUCUAGUGGUAGAAU | VEGFA_T8 | ATTATCAGCAA |
| tRNA_ | AGUACCCUGCCACGGUACAGACCCGG | | GTGGCTGCA |
| VEGFA_T8 | GUUCGAUUCCCGGCUGGAAAUAAAAA | | (SEQ ID NO: 74) |
| | UGAGGAUGUAGUCACGGAUUCAUUAU | | |
| | CAGGUUCUGGCUGCAGGGUGCCUGAU | | |
| | CUGUGCCAGGGUUAAGCAUGCUGCGU | | |
| | GAGGUGCAGCCACGAGGUGCGAAA | | |
| | GUACGCCGCAAUGUGCGGAAUUCGC | | |
| | ACGAAAGUUGGCGGAUUUCUUCGGA | | |
| | AAUCGAGCCAAUAAGCAUAUCCAUA | | |
| | UUAUCUGCGGAUGGAUGUGCACUGC | | |
| | AAGAUUAUCAGCAAGUGGCUGCAUUU | | |
| | UUUGCG (SEQ ID NO: 122) | | |

*Engineered scaffold sequences are in boldface

Approximately 16 hours prior to transfection, 25,000 HEK293T cells in DMEM/10% FBS+Pen/Strep (D10 media) were plated into each well of a 96-well plate. On the day of transfection, the cells were 70-90% confluent. For each well to be transfected, a mixture of Lipofectamine™ 2000 and Opti-MEM™ was prepared and incubated at room temperature for 5 minutes (Solution 1). After incubation, the Lipofectamine 2000™: Opti-MEM™ mixture was added to a separate mixture containing the corresponding nuclease-RT fusion plasmid, editing template RNA plasmid, and Opti-MEM™ (Solution 2). In the case of negative controls, the nuclease-RT fusion plasmid was not included in Solution 2. Solutions 1 and 2 were mixed by pipetting up and down, then incubated at room temperature for 25 minutes. Following incubation, the Solution 1 and 2 mixture was added dropwise to each well of a 96-well plate containing the cells. Approximately 72 hours post transfection, cells were trypsinized by adding TrypLE™ to the center of each well and incubating at 37° C. for approximately 5 minutes. D10 media was then added to each well and mixed to resuspend cells. The resuspended cells were centrifuged at 500 g for 10 minutes to obtain a pellet, and the supernatant was discarded. The cell pellet was then resuspended in QuickExtract™ buffer (Lucigen®), and cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. Round 2 PCR (PCR2) was performed to add Illumina adapters and indices. Reactions were then pooled and purified by column purification. Sequencing runs were performed using a 150 Cycle NextSeq 500/550 Mid or High Output v2.5 Kit.

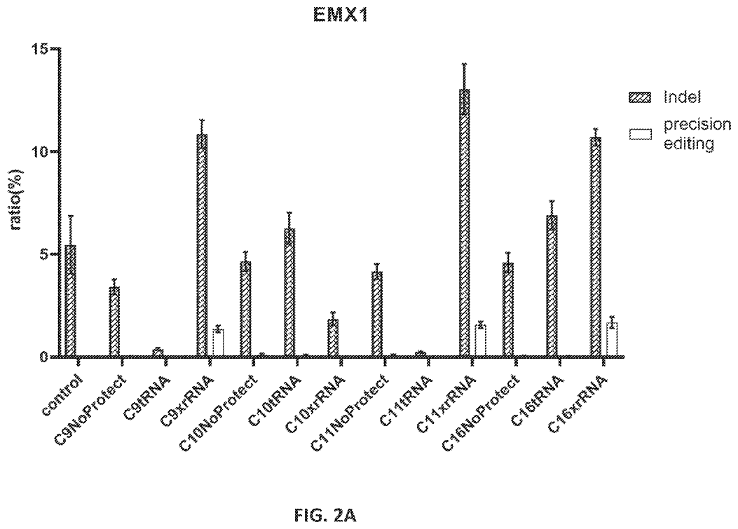

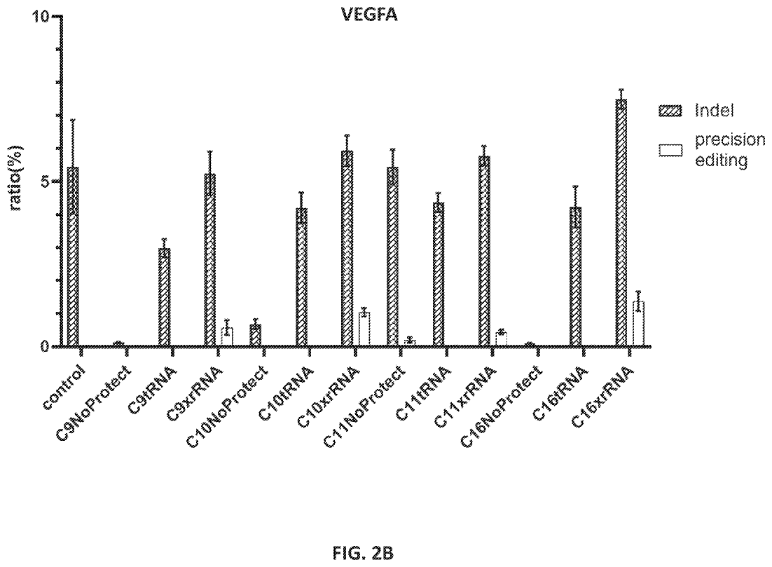

As shown in FIGS. 2A and 2B, the nuclease-RT fusion of SEQ ID NO: 5 and editing template RNA sequences introduced the encoded 4-nucleotide substitutions at the corresponding loci (white bars). The data shown is an average of two biological replicates, each of which had three technical replicates. Editing template RNAs engineered from guide RNA combos C9, C10, C11 and C16 resulted in higher indel activity on both targets (EMX1 and VEGFA, respectively), compared with the other engineered editing template RNAs (see raw data in Table 5 and Table 6). Precise editing efficiency for editing template RNAs that contained 3′end appended xrRNA (exoribonuclease-resistant RNAs) at both targets ranged from 1-2%, with an increase of 3-4 fold compared to other end protection strategies (no protection or tRNA). These editing template RNA also demonstrated significant increases in terms of indel ratio (filled bars) compared with the parent (1-2 fold increase). Overall, this example shows that the nuclease-RT fusion of SEQ ID NO: 5 introduced encoded edits into human genomic loci, and appending xrRNA at the 3′ terminus of editing template RNAs also drastically elevated the precision editing efficiency.

TABLE 5
Indel and precision editing efficiency of
editing template RNA variants on EMX1 loci

| Editing RNA Template | Average Indel | Average Precision |
|---|---|---|
| Variant | Ratio | Editing Ratio |
| control guide | 5.453% | 0.000% |
| C9_NoProtect_EMX1_TTTC | 3.410% | 0.031% |
| C9_tRNA_EMX1_TTTC | 0.392% | 0.001% |
| C9_xrRNA_EMX1_TTTC | 10.850% | 1.364% |
| C10_NoProtect_EMX1_TTTC | 4.647% | 0.104% |
| C10_tRNA_EMX1_TTTC | 6.263% | 0.070% |
| C10_xrRNA_EMX1_TTTC | 1.865% | 0.013% |
| C11_NoProtect_EMX1_TTTC | 4.163% | 0.078% |
| C11_tRNA_EMX1_TTTC | 0.239% | 0.000% |
| C11_xrRNA_EMX1_TTTC | 13.045% | 1.568% |
| C12_NoProtect_EMX1_TTTC | 14.631% | 0.249% |
| C12_tRNA_EMX1_TTTC | 0.175% | 0.000% |
| C12_xrRNA_EMX1_TTTC | 1.558% | 0.013% |
| C13_NoProtect_EMX1_TTTC | 4.471% | 0.065% |
| C13_tRNA_EMX1_TTTC | 0.233% | 0.000% |
| C13_xrRNA_EMX1_TTTC | 3.393% | 0.034% |
| C14_NoProtect_EMX1_TTTC | 8.054% | 0.136% |
| C14_tRNA_EMX1_TTTC | 2.297% | 0.004% |
| C14_xrRNA_EMX1_TTTC | 5.021% | 0.102% |
| C15_NoProtect_EMX1_TTTC | 3.682% | 0.038% |
| C15_tRNA_EMX1_TTTC | 5.710% | 0.022% |
| C15_xrRNA_EMX1_TTTC | 11.142% | 1.093% |
| C16_NoProtect_EMX1_TTTC | 4.591% | 0.047% |
| C16_tRNA_EMX1_TTTC | 6.893% | 0.025% |
| C16_xrRNA_EMX1_TTTC | 10.704% | 1.678% |

TABLE 6
Indel and precision editing efficiency of
editing template RNA variants on VEGFA loci

| Editing RNA Template | Average Indel | Average Precision |
|---|---|---|
| Variant | Ratio | Editing Ratio |
| control guide | 0.101% | 0.000% |
| C9_NoProtect_VEGFA_T8 | 0.127% | 0.000% |
| C9_tRNA_VEGFA_T8 | 2.982% | 0.004% |
| C9_xrRNA_VEGFA_T8 | 5.246% | 0.583% |
| C10_NoProtect_VEGFA_T8 | 0.683% | 0.000% |
| C10_tRNA_VEGFA_T8 | 4.199% | 0.003% |
| C10_xrRNA_VEGFA_T8 | 5.938% | 1.039% |
| C11_NoProtect_VEGFA_T8 | 5.448% | 0.198% |
| C11_tRNA_VEGFA_T8 | 4.372% | 0.006% |
| C11_xrRNA_VEGFA_T8 | 5.780% | 0.442% |
| C12_NoProtect_VEGFA_T8 | 3.901% | 0.049% |
| C12_tRNA_VEGFA_T8 | 4.289% | 0.008% |
| C12_xrRNA_VEGFA_T8 | 7.641% | 1.993% |
| C13_NoProtect_VEGFA_T8 | 4.028% | 0.037% |
| C13_tRNA_VEGFA_T8 | 0.992% | 0.000% |
| C13_xrRNA_VEGFA_T8 | 6.044% | 1.029% |
| C14_NoProtect_VEGFA_T8 | 3.659% | 0.018% |
| C14_tRNA_VEGFA_T8 | 4.019% | 0.033% |
| C14_xrRNA_VEGFA_T8 | 5.034% | 0.671% |
| C15_NoProtect_VEGFA_T8 | 0.331% | 0.000% |
| C15_tRNA_VEGFA_T8 | 3.941% | 0.005% |
| C15_xrRNA_VEGFA_T8 | 2.767% | 0.002% |
| C16_NoProtect_VEGFA_T8 | 0.103% | 0.000% |
| C16_tRNA_VEGFA_T8 | 4.231% | 0.014% |
| C16_xrRNA_VEGFA_T8 | 7.495% | 1.373% |

### Example 3—Engineering of Nuclease-RT Fusion Polypeptides for RNA-Templated Editing in HEK293T Cells

This Example describes indel and precision editing assessment on multiple targets using variant nuclease-RT fusions introduced into mammalian cells by transient transfection. The wild-type variant nuclease-RT fusion is SEQ ID NO: 5, and all mutations were on the nuclease of the nuclease-RT fusion polypeptide.

DNA templates comprising a single mutation (either arginine or glycine) were constructed via two PCR steps using mutagenic forward and mutagenic reverse primers ordered from IDT™ (Integrated DNA Technologies, Inc.). In the first step, two sets of PCR reactions were conducted in 384 plates to generate two fragments. The overlapping regions of two PCR fragments contained the desired single mutations and allowed the assembly of the entire DNA template via a second PCR. In the second step, the purified fragments from the first step were used as the template for the overlapping PCR (OL PCR) and the Fw and Rv oligos annealing to the vector backbone as the OL PCR primers. The resulting linear DNA templates contained a T7 promoter, a T7 terminator, and the open-reading frame for the polypeptide. The final library comprised 999 variants.

These linear DNA templates were used directly in a cell-free transcription and translation system to express the polypeptide variants with the intended single mutations. The variant constructs were further individually subcloned for transient transfection.

The editing template RNAs were synthesized and cloned as described in Example 2. The sequences are shown in Table 7.

TABLE 7
Mammalian Targets and Corresponding Editing Template RNAs

| Editing | | | |
|---|---|---|---|
| Template | | | |
| RNAs | Sequence | Target | Target Sequence |
| 1 | UGUCAGGCCUGCUAGUCAGCCACAGUUU | EMX1 | TGTTGCCCTCATAAC |
| | GGGGAAAGCUGUGCAGCCUGUAACCCCC | | TTATC (SEQ ID NO: 2) |
| | CCAGGAGAAGCUGGGAAAAACCGUCAUC | | |
| | AACAUUGCCUUUCUGUUGCCCAGUAAAC | | |
| | UUAUCAUACGGAGCCAGGCUAUAUAAUU | | |
| | AGCUACUUCUCCGUGAGGUGCAGCCACG | | |
| | AGGUGCGAAAGUACGCAGCAAUGUGCUG | | |
| | AAUUCGCACGAAAGUUGGCGGAUUUCUU | | |
| | CGGAAAUCGAGCCAAUAAUCAUAUCCAU | | |
| | AUUAUCUGCGGAUGGAUGUGAACUGCAA | | |
| | GUGUUGCCCUCAUAACUUAUC | | |
| | (SEQ ID NO: 123) | | |
| 2 | UGUCAGGCCUGCUAGUCAGCCACAGUUU | VEGFA | ATTATCAGCAAGTG |
| | GGGGAAAGCUGUGCAGCCUGUAACCCCC | | GCTGCA (SEQ ID |
| | CCAGGAGAAGCUGGGAAAAAUGAGGAU | | NO: 74) |
| | GUAGUCACGGAUUCAUUAUCAGGUUCUG | | |
| | GCUGCAGGGUGCCUGAUCUGUGCCAGGG | | |
| | UUAAGCAUGCUGCGUGAGGUGCAGCCAC | | |
| | GAGGUGCGAAAGUACGCAGCAAUGUGCU | | |
| | GAAUUCGCACGAAAGUUGGCGGAUUUCU | | |
| | UCGGAAAUCGAGCCAAUAAUCAUAUCCA | | |
| | UAUUAUCUGCGGAUGGAUGUGAACUGCA | | |
| | AGAUUAUCAGCAAGUGGCUGCA | | |
| | (SEQ ID NO: 124) | | |

Approximately 16 hours prior to transfection, 25,000 HEK293T cells in DMEM/10% FBS+Pen/Strep (D10 media) were plated into each well of a 96-well plate. On the day of transfection, the cells were 70-90% confluent. For each well to be transfected, a mixture of Lipofectamine™ 2000 and Opti-MEM™ was prepared and incubated at room temperature for 5 minutes (Solution 1). After incubation, the Lipofectamine 2000™:Opti-MEM™ mixture was added to a separate mixture containing a variant nuclease-RT fusion plasmid, editing template RNA plasmid, and Opti-MEM™ (Solution 2). In the case of negative controls, the editing template RNA plasmid was not included in Solution 2. Solutions 1 and 2 were mixed by pipetting up and down, then incubated at room temperature for 25 minutes. Following incubation, the Solution 1 and 2 mixture was added dropwise to each well of a 96-well plate containing the cells. Approximately 72 hours post transfection, cells were trypsinized by adding TrypLE™ to the center of each well and incubating at 37° C. for approximately 5 minutes. D10 media was then added to each well and mixed to resuspend cells. The resuspended cells were centrifuged at 500 g for 10 minutes to obtain a pellet, and the supernatant was discarded. The cell pellet was then resuspended in QuickExtract™ buffer (Lucigen®), and cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. Round 2 PCR (PCR2) was performed to add Illumina adapters and indices. Reactions were then pooled and purified by column purification. Sequencing runs were performed using a 150 Cycle NextSeq 500/550 Mid or High Output v2.5 Kit.

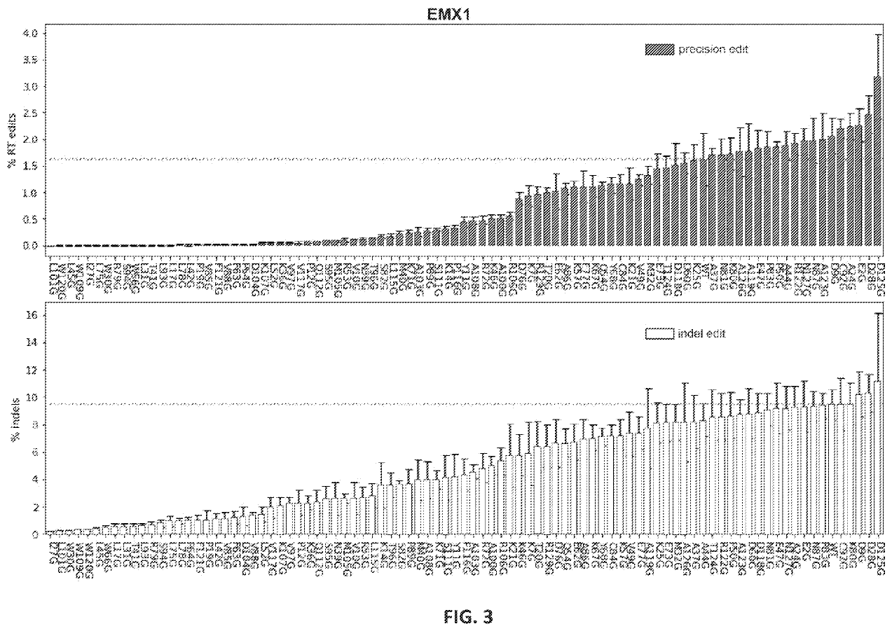

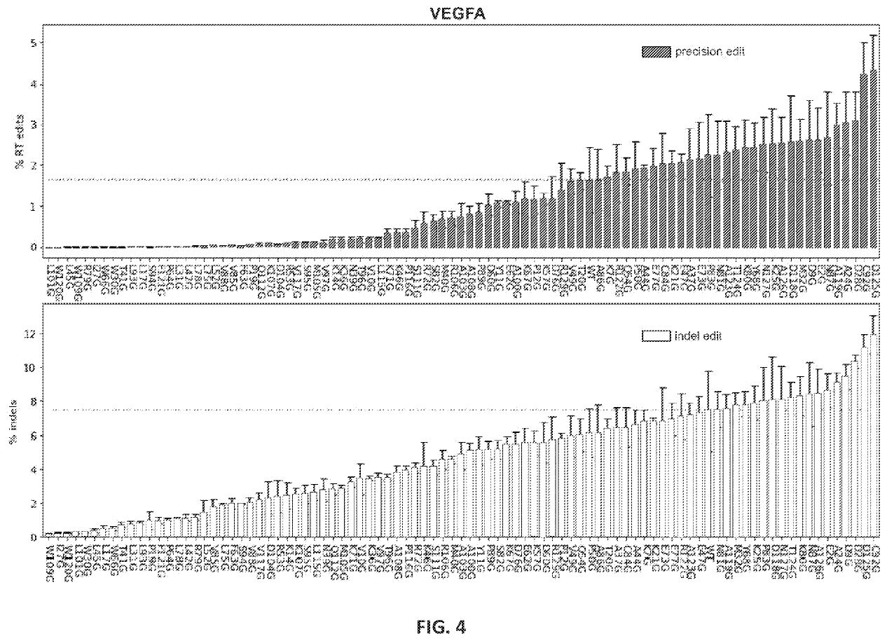

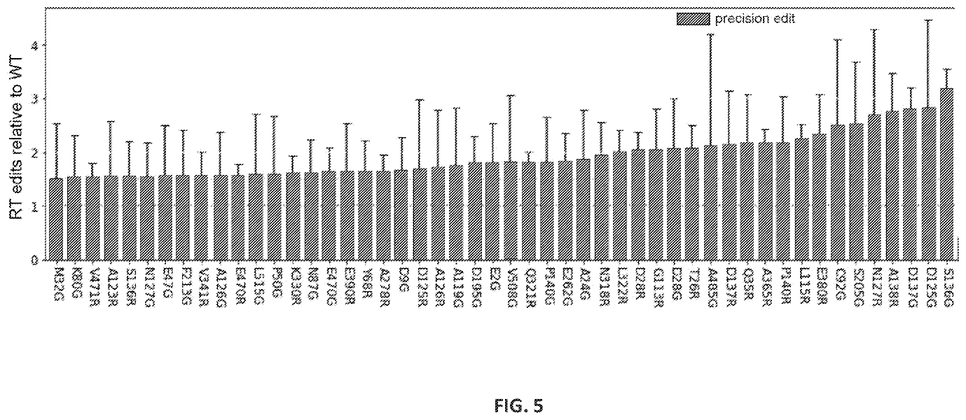

As shown in FIG. 3 and FIG. 4, various variant nuclease-RT fusion polypeptides comprising a single glycine or arginine substitution relative to the wild-type nuclease-RT fusion exhibited up to 2-4-fold increased indel activity (white bars). Precision editing activity (gray bars) was also increased at the EMX1 and VEGFA targets. The x-axis denotes the corresponding mutation for the nuclease of SEQ ID NO: 1. A full list of all single mutations engineered into the nuclease portion of the nuclease-RT fusions and their corresponding indel or precision editing ratios relative to wild-type (SEQ ID NO: 5) are summarized in Table 8. Overall, variant nuclease-RT fusion polypeptides comprising S136G, A138R, and/or D137G/R substitutions generally exhibited increased precise editing activities (approximately 2-3 fold higher) than other variant nuclease-RT fusion polypeptides (shown in FIG. 5).

This Example thus shows that the nuclease-RT fusion polypeptide of SEQ ID NO: 5 was capable of being successfully engineered to increase indel and precise editing activity.

TABLE 8
Editing Efficiencies of Nuclease-RT Fusion Polypeptide Variants

| 8218-RT Fusion | Average Indel Ratio to | Average RT editing |
|---|---|---|
| Variant | WT | Ratio to WT |
| V221G | 0 | 0.022676599 |
| L373R | 0 | 0.048174693 |
| L358R | 0 | 0.03921782 |
| R250G | 0.000129523 | 0.021910506 |
| A445R | 0.000291549 | 0.039816015 |
| I399R | 0.000446026 | 0.035946246 |
| C209G | 0.000502501 | 0.02322462 |
| E187G | 0.000504053 | 0.022228628 |
| S196G | 0.00054481 | 0.020528595 |
| E356R | 0.000565967 | 0.041745014 |
| Y252G | 0.000632913 | 0.023194089 |
| L381R | 0.000643465 | 0.054406104 |
| V353R | 0.000667062 | 0.039874151 |
| L422G | 0.000672777 | 0.034858584 |
| W120G | 0.000740823 | 0.037762839 |
| V251G | 0.000792096 | 0.022798513 |
| L218G | 0.000822937 | 0.022299968 |
| L101G | 0.000856265 | 0.03660137 |
| T215G | 0.000941998 | 0.022577658 |
| A214R | 0.001011255 | 0.04058481 |
| A244G | 0.001093082 | 0.021458632 |
| C431R | 0.001148624 | 0.035085231 |
| S447R | 0.00115544 | 0.036854813 |
| F505G | 0.001311717 | 0.049671867 |
| C409R | 0.001357301 | 0.046616126 |
| A444R | 0.001396551 | 0.049814731 |
| I216G | 0.001445867 | 0.023007191 |
| L45G | 0.001485971 | 0.054990204 |
| T406R | 0.001512437 | 0.057063108 |
| N443R | 0.001551822 | 0.033492944 |
| I448R | 0.001630398 | 0.040423015 |
| P255G | 0.001776595 | 0.024172819 |
| W109G | 0.001849937 | 0.033073813 |
| D441R | 0.00186926 | 0.037190571 |
| F429R | 0.001872806 | 0.042836219 |
| G269R | 0.001893613 | 0.042083534 |
| M378R | 0.001912833 | 0.041015608 |
| C434R | 0.001958975 | 0.037210811 |
| G239R | 0.001981779 | 0.065778996 |
| R335G | 0.002077234 | 0.049131694 |
| I482R | 0.002137636 | 0.078627627 |
| V514G | 0.002344306 | 0.062500042 |
| V346R | 0.002374829 | 0.04870628 |
| M364R | 0.002397466 | 0.099881036 |
| L385R | 0.002419953 | 0.049592226 |
| W120R | 0.002545222 | 0.102190752 |
| S343R | 0.002562178 | 0.041875096 |
| D375R | 0.002687688 | 0.062067674 |
| G235R | 0.002704099 | 0.042761959 |
| I236G | 0.00272979 | 0.057208376 |
| A389R | 0.002763578 | 0.071526669 |
| A376R | 0.002884428 | 0.06322736 |
| S247R | 0.003078448 | 0.042808583 |
| I27G | 0.00315592 | 0.033388945 |
| M363R | 0.003185101 | 0.056459657 |
| G277R | 0.003210511 | 0.116403631 |
| S143R | 0.003221533 | 0.037017838 |
| V88R | 0.003282391 | 0.048828967 |
| R79G | 0.003385194 | 0.133409702 |
| L324R | 0.003409691 | 0.091073837 |
| V240G | 0.003422578 | 0.057586025 |
| D237G | 0.00343365 | 0.037920782 |
| I27R | 0.003594516 | 0.128562242 |
| T491G | 0.003609088 | 0.090692653 |
| W30G | 0.003618955 | 0.040164153 |
| I216R | 0.003792659 | 0.071980947 |
| I507G | 0.003917978 | 0.061616375 |
| F493G | 0.003961646 | 0.076237636 |
| A404R | 0.00402735 | 0.08872132 |
| G98R | 0.004191191 | 0.195994363 |
| V204R | 0.004215557 | 0.033977968 |
| W66G | 0.00434701 | 0.069551086 |
| T41G | 0.004529525 | 0.090963081 |
| F505R | 0.005055024 | 0.096395509 |
| L164G | 0.005143731 | 0.079407717 |
| F402R | 0.00533147 | 0.091976805 |
| I448G | 0.005344363 | 0.173881164 |
| I162G | 0.005412796 | 0.058333841 |
| L93G | 0.005469792 | 0.10236503 |
| I257G | 0.00558313 | 0.087603371 |
| I153G | 0.005649178 | 0.077561486 |
| A350R | 0.005676106 | 0.073658664 |
| F146G | 0.005743986 | 0.078694671 |
| F402G | 0.005788565 | 0.241482527 |
| S94G | 0.005899369 | 0.191425376 |
| V234G | 0.006055146 | 0.095291634 |
| R141G | 0.006545776 | 0.108374174 |
| L17G | 0.006551178 | 0.072081331 |
| H492G | 0.007042533 | 0.119852929 |
| S405R | 0.007168097 | 0.041789532 |
| S201R | 0.007447839 | 0.090835364 |
| R165G | 0.007495913 | 0.124040445 |
| L31G | 0.008343614 | 0.092392425 |
| L369R | 0.008497552 | 0.079898153 |
| V204G | 0.008508041 | 0.086171315 |
| Y212G | 0.009245848 | 0.086565003 |
| W74G | 0.009879184 | 0.131662725 |
| F121G | 0.010400295 | 0.135751233 |
| L422R | 0.010684082 | 0.127904968 |
| F168G | 0.010718675 | 0.102652024 |
| C412R | 0.010751438 | 0.053099233 |
| A377R | 0.01097212 | 0.173469082 |
| L42G | 0.011393475 | 0.153858764 |
| L75G | 0.01232169 | 0.193885963 |
| P64G | 0.012332758 | 0.138706596 |
| V238R | 0.012739407 | 0.110630876 |
| F429G | 0.01276398 | 0.221144482 |
| V347R | 0.012910697 | 0.264374874 |
| I236R | 0.01307906 | 0.102640949 |
| R440G | 0.013201062 | 0.175471525 |
| V238G | 0.013452225 | 0.212489811 |
| L78G | 0.013702822 | 0.138656308 |
| I69R | 0.013868815 | 0.129125738 |
| T491R | 0.013925488 | 0.183501468 |
| L52R | 0.014134537 | 0.135557587 |
| F145G | 0.014523678 | 0.257962924 |
| C434G | 0.014723349 | 0.242804212 |
| S405G | 0.014741624 | 0.171677609 |
| T406G | 0.014799343 | 0.149102069 |
| N443G | 0.01483679 | 0.251650188 |
| L272R | 0.015634322 | 0.170878949 |
| A452R | 0.01596434 | 0.07429249 |
| S273R | 0.016046924 | 0.17266511 |
| I355R | 0.016520468 | 0.246001508 |
| I148G | 0.016922652 | 0.128284909 |
| H397R | 0.017406335 | 0.257414414 |
| D479G | 0.017919682 | 0.199649482 |
| C409G | 0.018013387 | 0.186553328 |
| H397G | 0.018038833 | 0.277939117 |
| V132G | 0.018178039 | 0.175015834 |
| Q407R | 0.018519871 | 0.124898351 |
| K388G | 0.018807818 | 0.354888072 |
| R408G | 0.019026379 | 0.205981394 |
| F486R | 0.019086289 | 0.225424975 |
| F490G | 0.019248999 | 0.196164764 |
| L449G | 0.019933502 | 0.185163829 |
| H340R | 0.021014662 | 0.314037322 |
| S374R | 0.0211393 | 0.217906559 |
| V489G | 0.021301129 | 0.295327732 |
| M331R | 0.021552378 | 0.153875019 |
| V170G | 0.021690262 | 0.14813296 |
| C431G | 0.02192468 | 0.226561452 |
| V85G | 0.021947746 | 0.205313074 |
| N254R | 0.022478391 | 0.186618058 |
| D509R | 0.022485759 | 0.21770638 |
| I69G | 0.022635852 | 0.155486848 |
| T403G | 0.022789979 | 0.264477135 |
| L416G | 0.023086083 | 0.349771466 |
| V88G | 0.023116743 | 0.230309817 |
| I507R | 0.023494268 | 0.139785746 |
| A445G | 0.023676965 | 0.367204465 |
| A100R | 0.023769925 | 0.313368224 |
| S494G | 0.023918886 | 0.410353985 |
| E356G | 0.024028652 | 0.221639406 |
| V360R | 0.025279443 | 0.190619623 |
| I15G | 0.025758722 | 0.152557871 |
| F486G | 0.025924097 | 0.392421197 |
| F63G | 0.026127935 | 0.222162798 |
| G483R | 0.02663373 | 0.25212633 |
| D517R | 0.027282257 | 0.225277289 |
| T217R | 0.027911929 | 0.2460719 |
| L45R | 0.028328686 | 0.140250287 |
| G99R | 0.029065571 | 0.196003283 |
| I399G | 0.029769327 | 0.275222153 |
| E328R | 0.029857741 | 0.172545764 |
| K480G | 0.030042503 | 0.454256796 |
| P19G | 0.030129899 | 0.144135326 |
| A404G | 0.030307465 | 0.39249136 |
| F490R | 0.032667883 | 0.299125948 |
| D237R | 0.032818931 | 0.169339407 |
| E328G | 0.032853057 | 0.237680383 |
| Y387G | 0.033207058 | 0.394330186 |
| K421G | 0.033392219 | 0.306326647 |
| L449R | 0.03448178 | 0.31224715 |
| C412G | 0.03479639 | 0.212618698 |
| I482G | 0.035045424 | 0.462759302 |
| L52G | 0.035684386 | 0.206109432 |
| D441G | 0.035741817 | 0.171920632 |
| I15R | 0.035977999 | 0.188825744 |
| R241G | 0.036205162 | 0.299994368 |
| W175R | 0.036827342 | 0.129214589 |
| F532G | 0.037600482 | 0.238490978 |
| I257R | 0.037820526 | 0.172988979 |
| A518R | 0.038131894 | 0.194158226 |
| S447G | 0.038263427 | 0.265482176 |
| A410R | 0.038750353 | 0.251485536 |
| C144R | 0.039112885 | 0.167737102 |
| V117R | 0.0391519 | 0.187196789 |
| N359G | 0.039241301 | 0.286612487 |
| T245R | 0.039782188 | 0.237962126 |
| V489R | 0.0399963 | 0.326458391 |
| Q339R | 0.04008765 | 0.261747411 |
| Y523R | 0.04019041 | 0.217326138 |
| K265G | 0.040233724 | 0.345119457 |
| A244R | 0.040286674 | 0.15923296 |
| V506R | 0.04061751 | 0.204631411 |
| R173G | 0.040723329 | 0.40047159 |
| V514R | 0.041215067 | 0.292855693 |
| D438R | 0.041382339 | 0.248650077 |
| G439R | 0.041470621 | 0.305708205 |
| K107G | 0.042354587 | 0.30932995 |
| D104G | 0.043062814 | 0.268879214 |
| F493R | 0.044009011 | 0.319903568 |
| W391G | 0.044122598 | 0.413753736 |
| R231G | 0.04444847 | 0.341523669 |
| L352G | 0.044818098 | 0.316799799 |
| L515R | 0.045021387 | 0.302661952 |
| V353G | 0.04539458 | 0.264098922 |
| K139G | 0.045406891 | 0.337909672 |
| D479R | 0.045647018 | 0.293137628 |
| F145R | 0.046696858 | 0.350256664 |
| L352R | 0.046764351 | 0.447340781 |
| K531G | 0.048279349 | 0.297521301 |
| Q112G | 0.048384791 | 0.346385921 |
| Y38G | 0.048431167 | 0.209297527 |
| Y38R | 0.049439199 | 0.263285315 |
| P19R | 0.050215344 | 0.202299592 |
| V451G | 0.052503209 | 0.419495416 |
| V117G | 0.052735317 | 0.277367445 |
| Q147G | 0.05299301 | 0.423163378 |
| P12R | 0.053482756 | 0.336428979 |
| R424G | 0.05367712 | 0.366481259 |
| Y396G | 0.054542811 | 0.458964054 |
| F283R | 0.055149547 | 0.441581131 |
| N516R | 0.055862596 | 0.335501549 |
| D246R | 0.056013282 | 0.322646128 |
| R133G | 0.056400774 | 0.417853305 |
| G520R | 0.056568881 | 0.293594177 |
| P481G | 0.057931904 | 0.542301346 |
| L101R | 0.058544314 | 0.138454278 |
| K361G | 0.059349704 | 0.369276331 |
| V97G | 0.061414801 | 0.388309706 |
| S95R | 0.062875713 | 0.17479721 |
| S95G | 0.06552941 | 0.342026317 |
| I456G | 0.067387625 | 0.399406023 |
| K388R | 0.069781531 | 0.489556591 |
| M105G | 0.070621872 | 0.352579537 |
| G400R | 0.071861109 | 0.288505776 |
| S392R | 0.072377269 | 0.415779274 |
| L200G | 0.072484021 | 0.252679514 |
| F261R | 0.07277594 | 0.393984938 |
| N503R | 0.073128666 | 0.28866497 |
| T401G | 0.073394735 | 0.446377253 |
| R53G | 0.077265732 | 0.350374515 |
| R270G | 0.079753087 | 0.433187657 |
| A398G | 0.081557546 | 0.588249637 |
| V251R | 0.08212071 | 0.216424497 |
| Y387R | 0.083878121 | 0.493719364 |
| L327R | 0.084326677 | 0.412415492 |
| V234R | 0.086612658 | 0.37636396 |
| I415G | 0.08683839 | 0.393938157 |
| K36G | 0.087710855 | 0.392574608 |
| F189R | 0.09032849 | 0.205676933 |
| L324G | 0.092888594 | 0.438563531 |
| D438G | 0.093900914 | 0.504425048 |
| P255R | 0.096958066 | 0.373480651 |
| M378G | 0.100563152 | 0.29538532 |
| K334R | 0.100803647 | 0.47831492 |
| V508R | 0.101299 | 0.298050184 |
| D509G | 0.103312163 | 0.398857821 |
| Y414R | 0.105071296 | 0.391471649 |
| M537G | 0.106747018 | 0.475526758 |
| K142G | 0.107141702 | 0.360267868 |
| L34G | 0.109211962 | 0.270999432 |
| A452G | 0.10934406 | 0.611069509 |
| S462G | 0.110292785 | 0.567012008 |
| T215R | 0.110854989 | 0.377459963 |
| V97R | 0.111980146 | 0.454806681 |
| W391R | 0.113042909 | 0.552683385 |
| M13G | 0.114033611 | 0.29338248 |
| A370G | 0.114326686 | 0.388201621 |
| K258G | 0.11433135 | 0.419756068 |
| V437G | 0.114799244 | 0.505943441 |
| K446G | 0.115297545 | 0.480222141 |
| V10G | 0.116232442 | 0.423562277 |
| K152G | 0.116874244 | 0.312627269 |
| T96G | 0.11945602 | 0.470460407 |
| A534G | 0.120205424 | 0.404218708 |
| W30R | 0.120694619 | 0.193310952 |
| R526G | 0.121122687 | 0.451849296 |
| D371R | 0.12327072 | 0.421982431 |
| I153R | 0.123652945 | 0.2078343 |
| D104R | 0.124300192 | 0.317690979 |
| N436R | 0.124867449 | 0.470590259 |
| V360G | 0.125655861 | 0.297507542 |
| A376G | 0.12660236 | 0.34783652 |
| A444G | 0.127164486 | 0.587021463 |
| Y477G | 0.128887032 | 0.566343046 |
| N39G | 0.129435936 | 0.369874139 |
| S462R | 0.131258135 | 0.458606437 |
| A529G | 0.132696506 | 0.391083557 |
| H492R | 0.133245275 | 0.492785722 |
| M243G | 0.133734907 | 0.280286293 |
| K498G | 0.133817006 | 0.397422482 |
| K484G | 0.13569431 | 0.289170652 |
| A354R | 0.136795742 | 0.544697984 |
| L75R | 0.137268763 | 0.359299469 |
| V347G | 0.138945829 | 0.452675203 |
| S379R | 0.139323807 | 0.522473587 |
| K279G | 0.139718221 | 0.353764583 |
| Y521R | 0.144936487 | 0.379248085 |
| M243R | 0.145219358 | 0.193565313 |
| N436G | 0.148410506 | 0.425868645 |
| G233R | 0.150154126 | 0.554983135 |
| L373G | 0.151891229 | 0.356828697 |
| A242G | 0.152039547 | 0.313083476 |
| V240R | 0.153047627 | 0.261921074 |
| P487G | 0.15343967 | 0.492317974 |
| Y342R | 0.15452956 | 0.540589208 |
| R383G | 0.156284254 | 0.479080869 |
| V221R | 0.156303065 | 0.299048447 |
| R223G | 0.156414384 | 0.485491518 |
| G249R | 0.157589316 | 0.437744029 |
| A108R | 0.157856125 | 0.282561465 |
| V460G | 0.159561528 | 0.633912747 |
| K467G | 0.159622813 | 0.669980285 |
| L115G | 0.161502955 | 0.361050827 |
| S247G | 0.163187726 | 0.365190743 |
| A394R | 0.163491573 | 0.523825555 |
| L42R | 0.164238413 | 0.262176256 |
| M363G | 0.16678153 | 0.294337761 |
| R417G | 0.167224705 | 0.560382832 |
| S201G | 0.168187796 | 0.480300476 |
| S143G | 0.168445377 | 0.495852428 |
| V428G | 0.170877042 | 0.556291745 |
| V160R | 0.175399978 | 0.32618977 |
| P64R | 0.176991853 | 0.273874412 |
| I8G | 0.178446042 | 0.445184221 |
| G362R | 0.178857282 | 0.61530997 |
| L218R | 0.179004675 | 0.315663376 |
| F168R | 0.179325339 | 0.334353843 |
| L381G | 0.179345464 | 0.423909512 |
| K14G | 0.18044025 | 0.405025744 |
| N254G | 0.182616561 | 0.312985712 |
| R203G | 0.182628889 | 0.44142054 |
| Q23R | 0.182818382 | 0.362233743 |
| R329G | 0.187703597 | 0.487616117 |
| E496G | 0.188284863 | 0.414608726 |
| P89R | 0.18982923 | 0.310853647 |
| V170R | 0.190123125 | 0.266067498 |
| I162R | 0.190571176 | 0.297726763 |
| P432R | 0.193772731 | 0.568814904 |
| S273G | 0.196927202 | 0.484503938 |
| P504R | 0.197244345 | 0.496673004 |
| D248R | 0.197602605 | 0.505844511 |
| E253R | 0.19771087 | 0.52625449 |
| K475G | 0.203227725 | 0.589123198 |
| G70R | 0.204510202 | 0.53952292 |
| K139R | 0.205196478 | 0.462263294 |
| K71G | 0.208808563 | 0.465795675 |
| Q407G | 0.20972413 | 0.559771185 |
| W74R | 0.210375205 | 0.366698155 |
| Y317G | 0.210804766 | 0.64516277 |
| G90R | 0.211086141 | 0.363978504 |
| W66R | 0.212608313 | 0.329159566 |
| T41R | 0.213067452 | 0.222451341 |
| A338G | 0.213986503 | 0.540141688 |
| P487R | 0.214398924 | 0.562824692 |
| E455G | 0.216326971 | 0.593510938 |
| Q519R | 0.217989662 | 0.452493307 |
| F121R | 0.218494352 | 0.274990763 |
| L369G | 0.218740391 | 0.391135236 |
| N357G | 0.221540551 | 0.444076026 |
| L34R | 0.222439975 | 0.344159347 |
| H535G | 0.225978211 | 0.564994284 |
| A370R | 0.226312603 | 0.537736864 |
| V451R | 0.227751055 | 0.49619939 |
| F146R | 0.228427903 | 0.263253254 |
| Q530G | 0.228882545 | 0.466279861 |
| S528G | 0.231069315 | 0.555457975 |
| Y18G | 0.232289321 | 0.599162284 |
| W109R | 0.232512641 | 0.275134389 |
| A278G | 0.233391466 | 0.435821154 |
| K468G | 0.23346016 | 0.520329847 |
| S111G | 0.237894867 | 0.537558448 |
| L164R | 0.238284755 | 0.302142731 |
| A37R | 0.241873996 | 0.290372802 |
| P116G | 0.241999461 | 0.535143547 |
| Q527G | 0.242010128 | 0.544298368 |
| V85R | 0.245186422 | 0.389931944 |
| L202G | 0.248233742 | 0.42014281 |
| L382R | 0.252995915 | 0.499030716 |
| A463G | 0.257303847 | 0.636076375 |
| A61R | 0.259827401 | 0.47865459 |
| Y414G | 0.261339951 | 0.674448559 |
| S494R | 0.261829221 | 0.492955315 |
| I179R | 0.263949737 | 0.461820309 |
| A377G | 0.266067735 | 0.534059151 |
| L202R | 0.266980452 | 0.306118174 |
| Y212R | 0.269600695 | 0.393964971 |
| R372G | 0.272583572 | 0.67987008 |
| P504G | 0.273855574 | 0.545798332 |
| P432G | 0.274155705 | 0.702678091 |
| F189G | 0.27586726 | 0.496686139 |
| Y5G | 0.277302563 | 0.451762998 |
| W175G | 0.278056326 | 0.562357378 |
| M105R | 0.279765451 | 0.267553544 |
| K171G | 0.282353848 | 0.575834851 |
| Y252R | 0.288244355 | 0.643557756 |
| Q519G | 0.289183675 | 0.434059142 |
| C92R | 0.290342946 | 0.291373238 |
| L200R | 0.29123968 | 0.431334252 |
| N65G | 0.293042877 | 0.550322899 |
| K258R | 0.293527641 | 0.544933958 |
| D426G | 0.293587238 | 0.701784026 |
| K384G | 0.29421697 | 0.504330948 |
| I148R | 0.294591689 | 0.389615879 |
| L78R | 0.295024526 | 0.348593347 |
| K114G | 0.29594939 | 0.522532495 |
| L358G | 0.296349374 | 0.393283392 |
| S94R | 0.297435152 | 0.375414921 |
| K536G | 0.298974754 | 0.620858732 |
| Y342G | 0.305559677 | 0.54541073 |
| T245G | 0.306621031 | 0.417187693 |
| K219G | 0.307887867 | 0.504430372 |
| S82G | 0.310868913 | 0.587201667 |
| D167R | 0.313265491 | 0.470283558 |
| S205R | 0.316708435 | 0.653697413 |
| Y43R | 0.31764964 | 0.683038108 |
| E495G | 0.318147132 | 0.689236165 |
| R333G | 0.319193469 | 0.71777466 |
| A463R | 0.319288226 | 0.555384557 |
| E496R | 0.319379051 | 0.621129825 |
| A103G | 0.319916089 | 0.592902354 |
| R332G | 0.321374516 | 0.804769426 |
| G33R | 0.323744329 | 0.549399101 |
| T225R | 0.324705008 | 0.623501932 |
| F190R | 0.327704303 | 0.482616442 |
| V199G | 0.328634311 | 0.714588282 |
| Q112R | 0.335870546 | 0.574657706 |
| A442R | 0.336650509 | 0.603726367 |
| G413R | 0.338497799 | 0.589337687 |
| M40G | 0.341719911 | 0.571570158 |
| A172R | 0.341985668 | 0.316186319 |
| G418R | 0.34340684 | 0.362835603 |
| P481R | 0.344112994 | 0.600530109 |
| V160G | 0.344661807 | 0.495256948 |
| K46G | 0.350781038 | 0.659821706 |
| V132R | 0.351164348 | 0.428983636 |
| K473G | 0.351716637 | 0.739269114 |
| M331G | 0.353704427 | 0.558595786 |
| D366R | 0.357198456 | 0.635398796 |
| G91R | 0.357237969 | 0.508445489 |
| H297G | 0.359296571 | 0.531465632 |
| Y193R | 0.361258812 | 0.480736566 |
| K384R | 0.364179324 | 0.76453878 |
| R275G | 0.366684861 | 0.497769895 |
| F63R | 0.368381348 | 0.572651374 |
| N525R | 0.371535941 | 0.74439406 |
| F181R | 0.372230325 | 0.47474841 |
| Q453G | 0.374072981 | 0.73012853 |
| R16G | 0.374578864 | 0.566465118 |
| W134G | 0.375634383 | 0.673276238 |
| N39R | 0.377486634 | 0.582904975 |
| E149R | 0.378953829 | 0.772587216 |
| R72G | 0.382676308 | 0.574441336 |
| F181G | 0.387123911 | 0.601036923 |
| A354G | 0.387197466 | 0.665582871 |
| T403R | 0.389464938 | 0.605481885 |
| L385G | 0.394199333 | 0.499374306 |
| K472G | 0.395999574 | 0.815273023 |
| L327G | 0.399370302 | 0.638440346 |
| Y477R | 0.399884178 | 0.66447033 |
| V346G | 0.405640233 | 0.580987797 |
| Q259G | 0.411452494 | 0.570598014 |
| P89G | 0.41391327 | 0.586518061 |
| D60R | 0.41892465 | 0.669192997 |
| N368R | 0.419528684 | 0.471642279 |
| I179G | 0.420119751 | 0.590629436 |
| D375G | 0.423191973 | 0.491454473 |
| P116R | 0.425913744 | 0.59981788 |
| L31R | 0.428732274 | 0.695053881 |
| D220R | 0.430527692 | 0.674708263 |
| S159R | 0.435508414 | 0.652075168 |
| S325R | 0.435917818 | 0.699704982 |
| S111R | 0.436483491 | 0.820814997 |
| P478G | 0.437210942 | 0.719429043 |
| Q339G | 0.437725577 | 0.69957096 |
| S427G | 0.437865052 | 0.941904054 |
| E345G | 0.438181589 | 0.670559984 |
| V433G | 0.438532573 | 0.780606411 |
| V430G | 0.438700394 | 0.767582129 |
| K349R | 0.439469147 | 0.663502143 |
| K334G | 0.439976351 | 0.67060783 |
| F261G | 0.440393505 | 0.675266478 |
| R106G | 0.441299092 | 0.617498338 |
| M364G | 0.442590377 | 0.464756838 |
| Y135G | 0.445933003 | 0.613358321 |
| Q271G | 0.447037784 | 0.638130769 |
| L268G | 0.449230764 | 0.879840302 |
| N488R | 0.451315677 | 0.68085756 |
| S56G | 0.451785835 | 0.606131664 |
| Y396R | 0.456326951 | 0.767226002 |
| A108G | 0.456911167 | 0.508654436 |
| N513G | 0.457317352 | 0.613605498 |
| D248G | 0.457728967 | 0.592259539 |
| W533G | 0.458880082 | 0.803289931 |
| N511G | 0.464640688 | 0.645826191 |
| I415R | 0.467697182 | 0.682718162 |
| Q147R | 0.46806021 | 0.713060848 |
| S367G | 0.468506728 | 0.661142141 |
| G459R | 0.469651358 | 0.748595792 |
| D366G | 0.47104239 | 0.639031914 |
| G174R | 0.473265526 | 0.72970608 |
| P12G | 0.475658718 | 0.566842101 |
| A442G | 0.476071182 | 0.893878684 |
| I131G | 0.479090506 | 0.634821173 |
| M40R | 0.480252642 | 0.680138323 |
| F190G | 0.486399661 | 0.634906747 |
| P461G | 0.487504626 | 0.727001163 |
| N420G | 0.488584804 | 0.796566247 |
| T124R | 0.489449067 | 0.595868486 |
| V428R | 0.492248315 | 0.69545372 |
| F293G | 0.494440245 | 0.876018591 |
| Y317R | 0.496303047 | 0.754256292 |
| S196R | 0.496905246 | 0.565027555 |
| K14R | 0.499681435 | 0.667510822 |
| T476G | 0.499682547 | 0.903125812 |
| R256G | 0.502457874 | 0.786815318 |
| Y18R | 0.503950856 | 0.733651203 |
| E495R | 0.504630797 | 0.76689189 |
| A350G | 0.507488853 | 0.602340675 |
| R316G | 0.509305877 | 0.737331889 |
| I355G | 0.513264173 | 0.64614274 |
| A344R | 0.51683167 | 0.68117226 |
| E419G | 0.517175427 | 0.789837969 |
| D9R | 0.517310278 | 0.761934047 |
| K349G | 0.518077338 | 0.641383227 |
| N294R | 0.522465822 | 0.771970706 |
| A242R | 0.525625354 | 0.632750663 |
| F283G | 0.527031429 | 0.640977069 |
| V10R | 0.530147119 | 0.743673864 |
| R206G | 0.530918419 | 0.742363378 |
| D426R | 0.534949518 | 0.630772965 |
| S82R | 0.536509985 | 0.667370654 |
| N280G | 0.537257048 | 0.858702007 |
| N6G | 0.54007889 | 0.799164506 |
| F293R | 0.540266461 | 0.764681183 |
| L93R | 0.540425287 | 0.429895049 |
| D207R | 0.543174504 | 0.837166989 |
| G522R | 0.543242686 | 0.806470139 |
| R155G | 0.548513541 | 0.661497331 |
| K198G | 0.549779333 | 0.60168695 |
| K46R | 0.551908516 | 0.979220808 |
| G59R | 0.553506123 | 0.804374834 |
| N503G | 0.559770327 | 0.708020846 |
| A61G | 0.560346974 | 0.839778648 |
| H130R | 0.560777727 | 0.744612799 |
| A518G | 0.562758168 | 0.632411509 |
| K288R | 0.564156312 | 0.666227889 |
| M13R | 0.569472953 | 0.597082156 |
| Y161G | 0.569740195 | 0.697212291 |
| S314R | 0.57503559 | 0.774214173 |
| T266G | 0.576550851 | 0.810755824 |
| Y43G | 0.576629566 | 0.805084842 |
| E510R | 0.578859525 | 0.769108564 |
| E184R | 0.581758734 | 0.694469945 |
| A100G | 0.582844393 | 0.642514414 |
| K166G | 0.584507619 | 0.695086551 |
| D246G | 0.586511108 | 0.695540359 |
| A344G | 0.591098407 | 0.730588809 |
| Y497G | 0.592408895 | 0.490707962 |
| T217G | 0.595170566 | 0.729269962 |
| I466G | 0.5967523 | 0.820811102 |
| R502G | 0.597866989 | 0.681192557 |
| K330G | 0.597939409 | 0.931783247 |
| T310G | 0.598028765 | 0.701124375 |
| A290R | 0.598863998 | 0.775415068 |
| Q259R | 0.599153811 | 0.765238681 |
| K454G | 0.599720768 | 0.886172427 |
| Q539G | 0.603705036 | 0.558359127 |
| D177R | 0.605855913 | 0.83375986 |
| A398R | 0.606719834 | 0.883186104 |
| R435G | 0.609575196 | 1.004739461 |
| Q54R | 0.610231423 | 0.620502499 |
| Q530R | 0.613382576 | 0.768939023 |
| R284G | 0.616814338 | 0.543512319 |
| S185G | 0.619706181 | 0.960421457 |
| N511R | 0.626885575 | 0.74713516 |
| E110R | 0.630021838 | 1.029710862 |
| K226G | 0.631306128 | 0.773985186 |
| K36R | 0.632987296 | 0.671419389 |
| Y11G | 0.634115752 | 0.636995407 |
| L17R | 0.63766615 | 0.463166669 |
| N302R | 0.638278491 | 0.829252744 |
| R169G | 0.638984869 | 0.742832906 |
| E230R | 0.642416599 | 0.7963435 |
| K197G | 0.643094092 | 0.710675756 |
| A389G | 0.645338464 | 1.035378633 |
| S374G | 0.645995162 | 0.770198373 |
| E298G | 0.646827812 | 0.760801802 |
| K197R | 0.647750774 | 0.776108276 |
| L268R | 0.64878819 | 0.788388076 |
| K323G | 0.65343989 | 0.80003156 |
| E228R | 0.65385537 | 0.773901169 |
| K265R | 0.654121079 | 0.748763988 |
| N516G | 0.654818333 | 0.888977712 |
| V199R | 0.655489944 | 0.928791028 |
| W134R | 0.657755396 | 0.797789678 |
| N359R | 0.657903054 | 0.822359178 |
| G128R | 0.663346452 | 0.625114821 |
| Y193G | 0.664696542 | 0.731012088 |
| A320G | 0.669873686 | 0.757953737 |
| E345R | 0.670727822 | 0.707303571 |
| R274G | 0.673033168 | 0.960644149 |
| Q453R | 0.678496191 | 0.801892896 |
| E156R | 0.678510812 | 0.873383125 |
| N176G | 0.680379151 | 0.78134477 |
| Y5R | 0.680834418 | 0.601373502 |
| K219R | 0.681673577 | 0.761156148 |
| K446R | 0.685722324 | 0.879561885 |
| Q319R | 0.68997661 | 0.885276412 |
| N208G | 0.69030639 | 0.825103379 |
| A150R | 0.691544817 | 0.817445701 |
| C287G | 0.692455922 | 0.705494438 |
| R180G | 0.695466427 | 0.860418497 |
| K386G | 0.695988646 | 0.657466955 |
| K260G | 0.698808638 | 0.959466528 |
| K281R | 0.700642439 | 0.867750589 |
| E298R | 0.701332496 | 0.875056412 |
| T474G | 0.701374092 | 0.929440232 |
| K281G | 0.705736987 | 0.948111321 |
| I456R | 0.705751894 | 0.859019564 |
| N420R | 0.708932716 | 0.854489934 |
| N302G | 0.709616573 | 0.74401064 |
| K178G | 0.711095642 | 0.781563978 |
| V306G | 0.711845591 | 0.715375694 |
| D76R | 0.712305504 | 0.732712149 |
| V227G | 0.712513633 | 0.852584606 |
| L465G | 0.714615758 | 0.801245932 |
| D177G | 0.716310598 | 0.807679083 |
| S395R | 0.717106545 | 0.884854774 |
| N318G | 0.717562666 | 0.697620351 |
| E187R | 0.718185313 | 0.753583698 |
| K480R | 0.722568028 | 0.831742401 |
| S58R | 0.72411922 | 0.866872693 |
| S325G | 0.727799231 | 1.195951279 |
| Y497R | 0.728311745 | 0.852963427 |
| K55G | 0.728589768 | 0.875207929 |
| R295G | 0.730109 | 1.063001957 |
| Q500G | 0.73028481 | 0.836995265 |
| E62R | 0.730889139 | 0.586780868 |
| K80R | 0.731686681 | 0.762274242 |
| G393R | 0.736714032 | 0.847084523 |
| P308R | 0.739056357 | 0.840631947 |
| E458R | 0.739805605 | 0.88119412 |
| Q35G | 0.741600357 | 0.839052638 |
| L312R | 0.742497654 | 0.931088152 |
| L305R | 0.743827105 | 0.917266935 |
| K142R | 0.74757714 | 0.731289321 |
| D76G | 0.74824866 | 0.771086591 |
| Q321G | 0.749918346 | 0.629544813 |
| Y11R | 0.749954286 | 0.632413263 |
| D118R | 0.751058325 | 0.545433669 |
| K57R | 0.751874425 | 0.643612968 |
| E183R | 0.752135513 | 0.832108113 |
| D371G | 0.753475217 | 0.90359739 |
| S159G | 0.755670245 | 0.892570141 |
| I301G | 0.756416587 | 1.015150237 |
| K152R | 0.759033137 | 0.867894814 |
| Q285R | 0.759842298 | 0.919359572 |
| E22R | 0.765735078 | 0.786032216 |
| Q23G | 0.767412166 | 0.839221969 |
| E51R | 0.767844751 | 0.869663267 |
| E191R | 0.76806534 | 0.757622176 |
| V306R | 0.768952874 | 0.91453255 |
| K260R | 0.775379333 | 0.833424838 |
| R129G | 0.777271197 | 0.740700565 |
| K25R | 0.777312386 | 0.617143642 |
| N368G | 0.778610534 | 0.739898845 |
| S395G | 0.778804529 | 0.963756778 |
| L469G | 0.779139781 | 0.992015276 |
| E110G | 0.779554961 | 1.06278734 |
| Y222R | 0.77970383 | 0.661846433 |
| K304G | 0.781271774 | 0.815424155 |
| E183G | 0.782689233 | 1.096822059 |
| V433R | 0.782888795 | 0.77809914 |
| S343G | 0.785964216 | 0.803501168 |
| D464G | 0.787325233 | 0.791509393 |
| E300R | 0.788006032 | 0.841141148 |
| E289G | 0.788683283 | 0.730075121 |
| K288G | 0.789093139 | 0.941939589 |
| N294G | 0.789474005 | 0.748090492 |
| A365G | 0.789539839 | 0.849538317 |
| M337G | 0.791821205 | 1.06229208 |
| Y222G | 0.795869113 | 0.808241982 |
| S351R | 0.799597993 | 0.986072889 |
| K326G | 0.803003219 | 0.764793597 |
| N6R | 0.803226709 | 0.799117746 |
| K67G | 0.803738584 | 0.793639542 |
| A485R | 0.805389371 | 0.760768538 |
| S457G | 0.809768632 | 0.967164582 |
| E62G | 0.809816382 | 0.801483003 |
| E419R | 0.811792301 | 0.794394579 |
| E303G | 0.812706615 | 0.829080387 |
| T96R | 0.812991937 | 1.016939349 |
| P461R | 0.813125894 | 0.930065427 |
| Y276G | 0.814883178 | 0.705151646 |
| L305G | 0.81609234 | 0.852519413 |
| K467R | 0.816401465 | 0.932967844 |
| K326R | 0.817449434 | 0.876872756 |
| K484R | 0.819145295 | 0.932366822 |
| T310R | 0.821935006 | 0.712998786 |
| P224R | 0.827395657 | 0.747941363 |
| E300G | 0.828216289 | 1.116248515 |
| R348G | 0.82927066 | 0.901670018 |
| S351G | 0.829407695 | 0.827555757 |
| S182R | 0.83011447 | 0.886185444 |
| K71R | 0.831335638 | 0.803703045 |
| K192G | 0.831671819 | 0.859669666 |
| K454R | 0.831734909 | 0.891766044 |
| E380G | 0.83446878 | 0.799958059 |
| A103R | 0.834702066 | 0.879928382 |
| L382G | 0.835645894 | 0.885311068 |
| E22G | 0.8386822 | 1.076166017 |
| Y161R | 0.844311588 | 0.996320376 |
| K361R | 0.846423538 | 0.887734231 |
| K264G | 0.847258248 | 0.98335455 |
| P50R | 0.848890616 | 0.781131246 |
| E184G | 0.851552513 | 1.009851663 |
| D195R | 0.852116422 | 0.59519771 |
| A44R | 0.853736072 | 0.659383835 |
| P313G | 0.85469778 | 0.87763853 |
| Q158R | 0.857386144 | 0.923853601 |
| K21R | 0.859500635 | 0.750939955 |
| K166R | 0.863334206 | 0.985087601 |
| V3G | 0.869278769 | 0.988274113 |
| L416R | 0.871466352 | 0.974429476 |
| A86R | 0.873403618 | 0.95960297 |
| K304R | 0.875657172 | 0.927282309 |
| V471G | 0.876665756 | 1.031151104 |
| V437R | 0.877681923 | 0.92767931 |
| S392G | 0.877891051 | 1.019305085 |
| K309G | 0.885338306 | 0.837839714 |
| E253G | 0.890771879 | 0.844589477 |
| R524G | 0.893930022 | 0.684447606 |
| C209R | 0.894372015 | 0.649614345 |
| D286G | 0.894494049 | 0.724212375 |
| K57G | 0.895058901 | 0.835777475 |
| R291G | 0.895782184 | 0.834343755 |
| D464R | 0.897903236 | 0.90881516 |
| A290G | 0.898934643 | 1.106013867 |
| K501R | 0.901260055 | 0.839065505 |
| N513R | 0.904172193 | 0.992092384 |
| I301R | 0.912487305 | 0.948122164 |
| A229R | 0.913830143 | 0.852666908 |
| Q282R | 0.91399897 | 0.969606418 |
| G151R | 0.918029407 | 1.169429439 |
| R315G | 0.921154411 | 1.173025631 |
| K386R | 0.921372812 | 0.976114329 |
| L296G | 0.92217351 | 0.852236232 |
| M32R | 0.924332792 | 0.789667014 |
| A119R | 0.92470021 | 0.711108211 |
| Q450G | 0.924929579 | 0.879013262 |
| N538G | 0.925215815 | 0.759149779 |
| V227R | 0.928782731 | 0.960738905 |
| K157R | 0.935075781 | 1.004724172 |
| E73R | 0.936410714 | 0.93604882 |
| K264R | 0.93826889 | 0.770187739 |
| K198R | 0.939656009 | 0.50914594 |
| V341G | 0.940350462 | 0.744436352 |
| Q319G | 0.940615421 | 1.20695428 |
| G48R | 0.942006214 | 0.766755262 |
| S292G | 0.946498766 | 1.177062298 |
| K323R | 0.948593489 | 0.951954696 |
| K192R | 0.948828556 | 1.049546921 |
| S314G | 0.949523454 | 0.899363133 |
| D425R | 0.95037231 | 0.843100772 |
| S379G | 0.951955183 | 1.049503212 |
| A24R | 0.952936365 | 1.099775938 |
| Q285G | 0.953328644 | 1.087745772 |
| C84R | 0.954795098 | 0.907086448 |
| D60G | 0.956146468 | 0.935793944 |
| S528R | 0.956829416 | 0.869512167 |
| D263R | 0.963209326 | 0.923646149 |
| K178R | 0.963277067 | 0.95793872 |
| L296R | 0.963723051 | 0.979328776 |
| L322G | 0.963968154 | 0.934758168 |
| P83R | 0.965939077 | 1.061455126 |
| E156G | 0.966148415 | 0.91959147 |
| T163G | 0.966460961 | 0.935372054 |
| A86G | 0.971445228 | 0.825463042 |
| I8R | 0.977220872 | 0.787621698 |
| F213R | 0.978447951 | 1.073854377 |
| N176R | 0.978823898 | 0.956703149 |
| V311G | 0.982904503 | 0.884619606 |
| K421R | 0.985210096 | 1.000933728 |
| K473R | 0.986065329 | 1.024195176 |
| S58G | 0.986706825 | 0.904197458 |
| E51G | 0.990218807 | 0.930941326 |
| P308G | 0.993043259 | 1.108863846 |
| M337R | 0.994973751 | 0.975587407 |
| G29R | 0.997503488 | 0.767683775 |
| WT | 1 | 1 |
| Q4R | 1.000650435 | 0.848117799 |
| P478R | 1.002453957 | 0.816339576 |
| A338R | 1.003100094 | 0.828170708 |
| K468R | 1.004180057 | 0.991818759 |
| V307R | 1.005854128 | 0.931461 |
| S154R | 1.007799092 | 1.106995418 |
| K501G | 1.011183152 | 1.004892996 |
| N81R | 1.011618018 | 0.895741507 |
| C144G | 1.012348865 | 0.955010872 |
| Q271R | 1.012670331 | 0.941750615 |
| S457R | 1.013776404 | 1.09606471 |
| V460R | 1.022955773 | 1.027832162 |
| E458G | 1.023669097 | 0.910584589 |
| K232G | 1.026531868 | 0.927348472 |
| L272G | 1.027109316 | 0.773305674 |
| E303R | 1.027784136 | 0.865808401 |
| K279R | 1.028653066 | 1.036391838 |
| Y135R | 1.031058732 | 1.111503772 |
| T26G | 1.032745202 | 1.001212576 |
| E47R | 1.035195976 | 1.001234596 |
| K7G | 1.045936589 | 0.852464798 |
| Q282G | 1.046774277 | 1.153430351 |
| E390G | 1.048348773 | 1.336853027 |
| T20G | 1.050932214 | 0.839648621 |
| E267R | 1.052950896 | 0.977778783 |
| S292R | 1.055503225 | 1.096997534 |
| P224G | 1.056066473 | 1.088260918 |
| K7R | 1.058389358 | 0.944673338 |
| K55R | 1.058561684 | 0.946951004 |
| D286R | 1.060968094 | 1.053719807 |
| Q158G | 1.064835511 | 1.005878379 |
| D517G | 1.069155416 | 0.946761362 |
| Q450R | 1.078438608 | 0.962547347 |
| T163R | 1.078661413 | 1.112258281 |
| V506G | 1.081866279 | 1.026816607 |
| A529R | 1.082949081 | 1.048861626 |
| Q500R | 1.088975401 | 1.011777857 |
| T225G | 1.089642797 | 0.958463342 |
| L312G | 1.090304893 | 1.239163817 |
| S299G | 1.093691768 | 1.12341423 |
| S427R | 1.095723521 | 0.858846439 |
| E77R | 1.096086353 | 1.025384103 |
| Y276R | 1.096979479 | 0.812562234 |
| K226R | 1.09759122 | 1.032960533 |
| K171R | 1.101749714 | 0.770736392 |
| Q4G | 1.105747542 | 0.921063678 |
| D186R | 1.108846114 | 1.095310754 |
| S185R | 1.109841074 | 1.074828533 |
| E289R | 1.110205157 | 1.002672577 |
| A138G | 1.118877275 | 1.025142612 |
| E455R | 1.124111962 | 0.941931563 |
| E2R | 1.124250354 | 1.030847274 |
| Y523G | 1.125119213 | 1.095207576 |
| K232R | 1.130504137 | 1.094642984 |
| E77G | 1.133501739 | 0.918251834 |
| N525G | 1.13469897 | 0.76415118 |
| C84G | 1.135332804 | 0.864313137 |
| V307G | 1.13750929 | 1.103590126 |
| A410G | 1.140460998 | 1.066403084 |
| V311R | 1.141588437 | 0.993693981 |
| K157G | 1.142060606 | 1.043188099 |
| E149G | 1.145612115 | 1.13810777 |
| A423G | 1.147769093 | 1.060794171 |
| K498R | 1.14809113 | 0.930376415 |
| V49R | 1.149625504 | 1.006421009 |
| D220G | 1.155611557 | 1.072595465 |
| N208R | 1.167883781 | 1.150567867 |
| N87R | 1.168518552 | 0.966942835 |
| D211R | 1.171724294 | 1.136843309 |
| D186G | 1.174499426 | 0.875462853 |
| K309R | 1.177554723 | 0.870423137 |
| E262R | 1.183660236 | 1.071553969 |
| A150G | 1.184365702 | 0.998886803 |
| S367R | 1.185241246 | 1.144666869 |
| K411R | 1.18667017 | 1.090908929 |
| V49G | 1.187807066 | 0.880689321 |
| D207G | 1.200388928 | 1.106696193 |
| H130G | 1.201963738 | 0.976009163 |
| N280R | 1.206173035 | 0.942269684 |
| D336R | 1.211105812 | 0.931917255 |
| E267G | 1.214013278 | 1.190176432 |
| A320R | 1.21893776 | 1.069424124 |
| K411G | 1.222249685 | 1.18201129 |
| Q54G | 1.224859956 | 0.842615906 |
| S299R | 1.232480811 | 0.89616017 |
| T266R | 1.234070571 | 0.963689261 |
| K472R | 1.241283318 | 0.942919165 |
| S182G | 1.243696086 | 1.121518835 |
| V3R | 1.244104301 | 0.828483287 |
| A394G | 1.246424629 | 1.173125347 |
| R122G | 1.249120036 | 0.9659947 |
| E73G | 1.252787382 | 0.944179203 |
| I131R | 1.258781331 | 1.213823503 |
| R194G | 1.270624063 | 1.031265259 |
| K475R | 1.285559373 | 1.050949969 |
| K21G | 1.28809336 | 0.836725418 |
| V430R | 1.289762903 | 1.058187101 |
| K114R | 1.292838632 | 1.010993776 |
| K512G | 1.297681424 | 0.92943169 |
| K67R | 1.297854738 | 1.023398202 |
| E228G | 1.300624028 | 1.065708435 |
| H297R | 1.317900999 | 1.038388013 |
| D211G | 1.322110195 | 1.016336843 |
| A229G | 1.327787632 | 1.077425145 |
| N357R | 1.332004158 | 1.169015345 |
| E230G | 1.335395824 | 1.172696196 |
| D118G | 1.34046512 | 1.019846556 |
| G102R | 1.340901634 | 0.953955491 |
| G188R | 1.341100581 | 0.923934782 |
| Y68G | 1.358490367 | 0.97054059 |
| C287R | 1.36010553 | 1.238688108 |
| L469R | 1.360661839 | 1.296660712 |
| T20R | 1.365497029 | 1.112252025 |
| T124G | 1.36893476 | 1.048339021 |
| D336G | 1.371033376 | 1.20594893 |
| N65R | 1.374753419 | 0.837389832 |
| I466R | 1.38374011 | 1.156140674 |
| A37G | 1.387523821 | 0.926421948 |
| K107R | 1.389456091 | 1.049602957 |
| L465R | 1.389635497 | 1.205370255 |
| D167G | 1.401343076 | 1.20628286 |
| K512R | 1.401712522 | 1.059690554 |
| A214G | 1.402880383 | 1.16816231 |
| K499G | 1.404424228 | 1.029023151 |
| T474R | 1.40814644 | 1.196959573 |
| E510G | 1.414349731 | 1.108397479 |
| N81G | 1.41513389 | 1.055283521 |
| P83G | 1.431172834 | 1.102425216 |
| Q527R | 1.432807155 | 0.927578802 |
| E191G | 1.437026456 | 1.150140337 |
| S154G | 1.440006865 | 1.151882542 |
| Y521G | 1.455486082 | 1.082147702 |
| H340G | 1.460580471 | 1.384396227 |
| G210R | 1.46525526 | 1.042596224 |
| A123G | 1.466149374 | 0.99311302 |
| N488G | 1.470144026 | 1.092687786 |
| S56R | 1.474392244 | 1.051761092 |
| A423R | 1.479723624 | 1.13861427 |
| K499R | 1.481869833 | 1.022560681 |
| D425G | 1.482515085 | 1.168791613 |
| T476R | 1.490691956 | 1.043873831 |
| A172G | 1.494938355 | 1.214092495 |
| A44G | 1.495139296 | 0.981519796 |
| T401R | 1.495898833 | 1.160279132 |
| K25G | 1.498370167 | 1.031709839 |
| D263G | 1.505443767 | 1.455995305 |
| P313R | 1.506544645 | 1.022505666 |
| M32G | 1.510971688 | 1.029456909 |
| K80G | 1.542895144 | 1.148082618 |
| V471R | 1.542976637 | 1.066721974 |
| A123R | 1.544111612 | 1.170281084 |
| S136R | 1.54816897 | 1.165271727 |
| N127G | 1.557494421 | 1.05370327 |
| E47G | 1.558806116 | 1.078726309 |
| F213G | 1.564542789 | 1.249597584 |
| V341R | 1.567934403 | 0.808442441 |
| A126G | 1.572631733 | 1.041341693 |
| E470R | 1.578314854 | 1.264078418 |
| L515G | 1.584663267 | 1.574598829 |
| P50G | 1.588440394 | 0.975937654 |
| K330R | 1.615627055 | 1.040600519 |
| N87G | 1.619764974 | 1.119467032 |
| E470G | 1.6267891 | 1.224566065 |
| E390R | 1.629187377 | 1.141385752 |
| Y68R | 1.63856889 | 0.949324291 |
| A278R | 1.639957077 | 1.160169352 |
| D9G | 1.662757166 | 1.271519089 |
| D125R | 1.688913602 | 1.248116417 |
| A126R | 1.727874542 | 1.218970214 |
| A119G | 1.768683333 | 0.966149382 |
| D195G | 1.78819279 | 1.294468691 |
| E2G | 1.792504656 | 1.155830688 |
| V508G | 1.796073854 | 1.157751811 |
| Q321R | 1.819423937 | 1.33182671 |
| P140G | 1.820907586 | 1.203645857 |
| E262G | 1.844587043 | 1.712003943 |
| A24G | 1.85773763 | 1.154521082 |
| N318R | 1.946165848 | 1.2839229 |
| L322R | 2.002895311 | 1.315983495 |
| D28R | 2.039802947 | 1.122308506 |
| G113R | 2.043356598 | 1.201005479 |
| D28G | 2.060297004 | 1.349417405 |
| T26R | 2.091141748 | 1.701570512 |
| A485G | 2.131949504 | 1.427236612 |
| D137R | 2.13488464 | 1.392046029 |
| Q35R | 2.167869563 | 1.433194427 |
| A365R | 2.182751466 | 1.282869771 |
| P140R | 2.190286034 | 1.712093891 |
| L115R | 2.233568178 | 1.441859633 |
| E380R | 2.336993964 | 1.743856347 |
| C92G | 2.476891434 | 1.402855418 |
| S205G | 2.531287938 | 1.654358653 |
| N127R | 2.695115077 | 1.507281121 |
| A138R | 2.771258696 | 1.266126091 |
| D137G | 2.802595885 | 1.664882146 |
| D125G | 2.83083855 | 1.435829756 |
| S136G | 3.181020402 | 1.907290176 |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the fore-going embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A gene editing system comprising:
(a) a polypeptide comprising a Type V CRISPR nuclease or a first nucleic acid encoding the polypeptide; wherein the Type V CRISPR nuclease comprises an amino acid at least 95% identical to SEQ ID NO: 1; and
(b) an engineered guide RNA (gRNA) or a second nucleic acid encoding the engineered gRNA, wherein the engineered gRNA comprises:
(i) a spacer sequence specific to a target sequence within a genomic site of interest, and
(ii) an engineered scaffold sequence, which is recognizable by the Type V CRISPR nuclease, wherein the engineered scaffold sequence comprises one or more mutations relative to a scaffold sequence comprising the nucleotide sequence of SEQ ID NO: 71; optionally wherein the engineered scaffold is at least 80% identical to SEQ ID NO: 71;
optionally wherein the spacer sequence is located at the 3′ of the scaffold sequence.

2. The gene editing system of claim 1, wherein the engineered scaffold sequence comprises one or more of the following mutations relative to the wide-type counterpart:
(i) nucleotide substitution at one or more of positions 25, 26, 30, 38, 48, 52, 55, 66, 67, 79, 82-87, 91-93, 96, 98-100, 104, 105, 107, 110, 113, 115, 118-121, 123 of SEQ ID NO: 71;
(ii) one or more deletions at positions of 25-31, 52-55, and 84-87 of SEQ ID NO: 71; and
(iii) one or more mutation within positions 72-77 of SEQ ID NO: 71; wherein the one or more mutations comprise nucleotide substitutions, deletions, insertions, or a combination thereof.

3. The gene editing system of claim 2, wherein the engineered scaffold sequence further comprises (iv) an insertion at 5′ end, between positions 107 and 108, and/or between positions 114 and 115.

4. The gene editing system of claim 1, wherein the engineered scaffold sequence is selected from those listed in Table 2 and Table 4; optionally wherein the engineered scaffold sequence is one of C9, C10, C11, and C16.

5. The gene editing system of claim 1, wherein the engineered scaffold sequence is about 115-135 nucleotides in length.

6. The gene editing system of claim 1, wherein the gene editing system further comprises:
(c) a reverse transcriptase (RT) or a third nucleic acid encoding the RT; and
(d) a reverse transcription donor RNA (RT donor RNA) or a fourth nucleic acid encoding the RT donor RNA, wherein the RT donor RNA comprises a primer binding site (PBS) and a template sequence.

7. The gene editing system of claim 6, wherein the RT is Moloney Murine Leukemia Virus (MMLV)-RT, mouse mammary tumor virus (MMTV)-RT, Marathon-RT, or RTx-RT.

8. The gene editing system of claim 6, wherein the Type V CRISPR nuclease and the RT form a fusion polypeptide.

9. The gene editing system of claim 8, wherein the fusion polypeptide comprises one or more mutations in the Type V CRISPR nuclease relative to the wild-type counterpart; and wherein the fusion polypeptide exhibits enhanced editing activity relative to the wild-type counterpart.

10. The gene editing system of claim 9, wherein the fusion polypeptide comprises the one or more amino acid substitutions depicted in FIG. 5; optionally wherein the fusion polypeptide comprises S136G, A138R, D137G, and/or D137R substitutions in the Type V CRISPR nuclease relative to SEQ ID NO: 1.

11. The gene editing system of claim 1, which comprises the Type V CRISPR nuclease, and the RT, which optionally form a fusion polypeptide.

12. The gene editing system of claim 1, which comprises at least one nucleic acid that expresses the Type V CRISPR nuclease and/or the RT; optionally wherein the gene editing system comprises a nucleic acid that expresses a fusion polypeptide comprising the Type V CRISPR and the RT.

13. The gene editing system of claim 12, wherein the at least one nucleic acid is a vector, which optionally is a viral vector.

14. The gene editing system of claim 13, wherein the at least one nucleic acid is a messenger RNA.

15. The gene editing system of claim 6, wherein the PBS in the RT donor RNA is 10-60-nucleotide in length, optionally 20-40-nucleotide in length.

16. The gene editing system of claim 15, wherein the PBS binds a PBS-targeting site that is adjacent to the complementary region of the target sequence, and wherein the PBS-targeting site is upstream to the complementary region of the target sequence.

17. The gene editing system of claim 6, wherein the template sequence is 5-100-nucleotide in length, optionally 30-50-nucleotide in length.

18. The gene editing system of claim 17, wherein the template sequence is homologous to the genomic site of interest and comprises one or more nucleotide variations relative to the genomic site of interest.

19. The gene editing system of claim 6, wherein the engineered gRNA and the RT donor RNA are located on a single RNA molecule; optionally wherein the engineered gRNA and the RT donor RNA are connected via a nucleotide linker.

20. The gene editing system of claim 1, wherein the system comprises one or more lipid nanoparticles (LNPs), which are associated with one or more of elements (a)-(d) of the system.

21. The gene editing system of claim 20, wherein the one or more LNPs are associated with up to three elements of (a)-(d), and wherein the system comprises at least one vector that expresses the remaining element(s).

22. A gene editing system comprising:
(a) a fusion polypeptide comprising a Type V CRISPR nuclease and a reverse transcriptase (RT), or a first nucleic acid encoding the fusion polypeptide;
(b) an engineered guide RNA (gRNA) or a second nucleotide encoding the engineered gRNA; and
(c) a reverse transcription donor RNA (RT donor RNA) or a fourth nucleic acid encoding the RT donor RNA, wherein the RT donor RNA comprises a primer binding site (PBS) and a template sequence;
wherein the fusion polypeptide comprises one or more mutations in the Type V CRISPR nuclease relative to the wild-type counterpart; and wherein the fusion polypeptide exhibits enhanced editing activity relative to the wild-type counterpart.

23. The gene editing system of claim 22, wherein the fusion polypeptide comprises the one or more amino acid substitutions depicted in FIG. 5; optionally wherein the fusion polypeptide comprises S136G, A138R, D137G, and/or D137R substitutions in the Type V CRISPR nuclease relative to SEQ ID NO: 1.

24. The gene editing system of claim 22, which comprises a nucleic acid that expresses the fusion polypeptide.

25. The gene editing system of claim 24, wherein the nucleic acid is a vector, which optionally is a viral vector.

26. The gene editing system of claim 24, wherein the nucleic acid expressing the fusion polypeptide is a messenger RNA.

27. The gene editing system of claim 22, wherein the PBS in the RT donor RNA is 10-60-nucleotide in length, optionally 20-40-nucleotide in length.

28. The gene editing system of claim 27, wherein the PBS binds a PBS-targeting site that is adjacent to the complementary region of the target sequence, and wherein the PBS-targeting site is upstream to the complementary region of the target sequence.

29. The gene editing system of claim 22, wherein the template sequence is 5-100-nucleotide in length, optionally 30-50-nucleotide in length.

30. The gene editing system of claim 29, wherein the template sequence is homologous to the genomic site of interest and comprises one or more nucleotide variations relative to the genomic site of interest.

31. The gene editing system of claim 22, wherein the engineered gRNA and the RT donor RNA are located on a single RNA molecule; optionally wherein the engineered gRNA and the RT donor RNA are connected via a nucleotide linker.

32. The gene editing system of claim 22, wherein the system comprises one or more lipid nanoparticles (LNPs), which are associated with one or more of elements (a)-(c) of the system.

33. The gene editing system of claim 32, wherein the one or more LNPs are associated with up to two elements of (a)-(c), and wherein the system comprises at least one vector that expresses the remaining element(s).

34. A pharmaceutical composition comprising the gene editing system of claim 1.

35. A kit comprising the elements of the gene editing system set forth in claim 1.

36. A method for genetically editing a cell, the method comprising contacting a host cell the gene editing system of claim 1 or the pharmaceutical composition comprising such to genetically edit the host cell.

37. The method of claim 36, wherein the host cell is cultured in vitro.

38. The method of claim 36, wherein the contacting step is performed by administering the gene editing system to a subject comprising the host cell.

39. A polynucleotide, comprising
(a) an engineered guide RNA (gRNA), which comprises:
(i) a spacer sequence specific to a target sequence within a genomic site of interest and
(ii) an engineered scaffold sequence, which is recognizable by a Type V CRISPR nuclease, wherein the engineered scaffold sequence comprises one or more mutations relative to the wild-type counterpart, which comprises the nucleotide sequence of SEQ ID NO: 71;
optionally wherein the engineered scaffold sequence is at least 80% identical to SEQ ID NO: 71.

40. The polynucleotide of claim 39, wherein the spacer sequence is located at the 3′ end of the scaffold sequence.

41. The polynucleotide of claim 39, wherein the engineered scaffold sequence comprises one or more of the following mutations relative to the wide-type counterpart:
(i) nucleotide substitution at one or more of positions 25, 26, 30, 38, 48, 52, 55, 66, 67, 79, 82-87, 91-93, 96, 98-100, 104, 105, 107, 110, 113, 115, 118-121, 123 of SEQ ID NO: 71;
(ii) one or more deletions at positions of 25-31, 52-55, and 84-87 of SEQ ID NO: 71; and
(iii) one or more mutation within positions 72-77 of SEQ ID NO: 71; wherein the one or more mutations comprise nucleotide substitutions, deletions, insertions, or
a combination thereof.

42. The polynucleotide of claim 41, wherein the engineered scaffold sequence further comprises (iv) an insertion at 5′ end, between positions 107 and 108, and/or between positions 114 and 115.

43. The polynucleotide of claim 39, wherein the engineered scaffold sequence is selected from those listed in Table 2 and Table 4; optionally wherein the engineered scaffold sequence is one of C9, C10, C11, and C16.

44. The polynucleotide of claim 39,
wherein the engineered scaffold sequence is about 115-135 nucleotides in length.

45. The polynucleotide of claim 39, which further comprises: (b) a reverse transcriptase template (RTT) RNA, optionally wherein (b) is located upstream to (a).

46. The polynucleotide of claim 45, which further comprises a nucleotide linker between (a) and (b), optionally wherein the nucleotide linker is a polyA linker.

47. The polynucleotide of claim 39, which further comprises a 5′ end U6 start fragment, an end protection fragment, a 3′ end U6 termination fragment, or a combination thereof.

48. A fusion polypeptide comprising a Type V CRISPR nuclease and a reverse transcriptase (RT), wherein the fusion polypeptide comprises one or more mutations in the Type V CRISPR nuclease relative to the wild-type counterpart; and wherein the fusion polypeptide exhibits enhanced editing activity relative to the wild-type counterpart.

49. The fusion polypeptide of claim 48, wherein the fusion polypeptide comprises the one or more amino acid substitutions depicted in FIG. 5; optionally wherein the fusion polypeptide comprises S136G, A138R, D137G, and/or D137R substitutions in the Type V CRISPR nuclease relative to SEQ ID NO: 1.


SEQUENCE LISTING (124 sequences)

SEQ ID NO: 1 - protein, 539 aa, synthetic construct
MEVQYNKIDVYPMKIRLYPTKEQAKTIDGWLMGLQKAYNMTLYALKEGVPELRQKSKSGDAEFPNWKYIGKREWLDELRKNSPCVANVPGGCLSSTVGGALGADMRKAWESQGKLPVDAWFRATDANGRHIVRWYSDAKPRKSCFFQIEAGKISREKQSVYITLRKDFRVKARGWNDKIRFSEESDEGFFEKYRDSKKVLSLRVSRDNCGDYFATITLKDVYRPTKVEAERKGVGIDVGVRAMATDSDGRVYENPRIKQKFEDKKTELGRQLSRRYGAKNKQFRQDCKEARSFNRLHESEINEKLVVPKTVLPSRRYNQAQLKLSKLERKMRRKRDMAQHVYSAEVVRKASLVAIENLNVKGMMADSNLADRLSDAAMSELLRKLKYKAEWSGASYHAIGTFTASTQRCAKCGYILRGENKLARDDSVFVCPVCRNVDGRDANAAKSILQVAQKEISEGVPSADLIKKLEVKKTKTYPDKPIGKAFPNVFTHFSEEYKKQKRNPFVIVDENKNVLNDAQGYGYRNRQSAQKFWAHKMNQ

SEQ ID NO: 2 - DNA, 20 bp, synthetic construct
  source 1..20: mol_type = other DNA
tgttgccctcataacttatc

SEQ ID NO: 3 - RNA, 20 nt, synthetic construct
  source 1..20: mol_type = other RNA
tgttgccctcataacttatc

SEQ ID NO: 4 - protein, 569 aa, synthetic construct
MKIEEGKGHHHHHHMEVQYNKIDVYPMKIRLYPTKEQAKTIDGWLMGLQKAYNMTLYALKEGVPELRQKSKSGDAEFPNWKYIGKREWLDELRKNSPCVANVPGGCLSSTVGGALGADMRKAWESQGKLPVDAWFRATDANGRHIVRWYSDAKPRKSCFFQIEAGKISREKQSVYITLRKDFRVKARGWNDKIRFSEESDEGFFEKYRDSKKVLSLRVSRDNCGDYFATITLKDVYRPTKVEAERKGVGIDVGVRAMATDSDGRVYENPRIKQKFEDKKTELGRQLSRRYGAKNKQFRQDCKEARSFNRLHESEINEKLVVPKTVLPSRRYNQAQLKLSKLERKMRRKRDMAQHVYSAEVVRKASLVAIENLNVKGMMADSNLADRLSDAAMSELLRKLKYKAEWSGASYHAIGTFTASTQRCAKCGYILRGENKLARDDSVFVCPVCRNVDGRDANAAKSILQVAQKEISEGVPSADLIKKLEVKKTKTYPDKPIGKAFPNVFTHFSEEYKKQKRNPFVIVDENKNVLNDAQGYGYRNRQSAQKFWAHKMNQKRPAATKKAGQAKKKK

SEQ ID NO: 5 - protein, 1297 aa, synthetic construct
MKIEEGKGHHHHHHMEVQYNKIDVYPMKIRLYPTKEQAKTIDGWLMGLQKAYNMTLYALKEGVPELRQKSKSGDAEFPNWKYIGKREWLDELRKNSPCVANVPGGCLSSTVGGALGADMRKAWESQGKLPVDAWFRATDANGRHIVRWYSDAKPRKSCFFQIEAGKISREKQSVYITLRKDFRVKARGWNDKIRFSEESDEGFFEKYRDSKKVLSLRVSRDNCGDYFATITLKDVYRPTKVEAERKGVGIDVGVRAMATDSDGRVYENPRIKQKFEDKKTELGRQLSRRYGAKNKQFRQDCKEARSFNRLHESEINEKLVVPKTVLPSRRYNQAQLKLSKLERKMRRKRDMAQHVYSAEVVRKASLVAIENLNVKGMMADSNLADRLSDAAMSELLRKLKYKAEWSGASYHAIGTFTASTQRCAKCGYILRGENKLARDDSVFVCPVCRNVDGRDANAAKSILQVAQKEISEGVPSADLIKKLEVKKTKTYPDKPIGKAFPNVFTHFSEEYKKQKRNPFVIVDENKNVLNDAQGYGYRNRQSAQKFWAHKMNQKRPAATKKAGQAKKKKSGGSSGGSSGSETPGTSESATPESSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPMKRTADGSEFESPKKKRKV

SEQ ID NO: 6 - RNA, 127 nt, synthetic construct
  source 1..127: mol_type = other RNA
ggtgcagccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 7 - RNA, 125 nt, synthetic construct
  source 1..125: mol_type = other RNA
tgcagccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 8 - RNA, 122 nt, synthetic construct
  source 1..122: mol_type = other RNA
agccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 9 - RNA, 121 nt, synthetic construct
  source 1..121: mol_type = other RNA
gccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 10 - RNA, 120 nt, synthetic construct
  source 1..120: mol_type = other RNA
ccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 11 - RNA, 119 nt, synthetic construct
  source 1..119: mol_type = other RNA
cacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 12 - RNA, 118 nt, synthetic construct
  source 1..118: mol_type = other RNA
acgaggtgcgaataggaagtacgcagcaatgtgctgaatcgttcgcacgaaaattggcattattgaaaaccaatgccaataatcatatccataaatatgtgtggttatgaactgcaag

SEQ ID NO: 13 - RNA, 117 nt, synthetic construct
  source 1..117: mol_type = other RNA
cgaggtgcgaataggaagtacgcagcaatgtgctgaatcgttcgcacgaaaattggcattattgaaaaccaatgccaataatcatatccataaatatgtgtggttatgaactgcaag

SEQ ID NO: 14 - RNA, 116 nt, synthetic construct
  source 1..116: mol_type = other RNA
gaggtgcgaataggaagtacgcagcaatgtgctgaatcgttcgcacgaaaattggcattattgaaaaccaatgccaataatcatatccataaatatgtgtggttatgaactgcaag

SEQ ID NO: 15 - RNA, 115 nt, synthetic construct
  source 1..115: mol_type = other RNA
aggtgcgaataggaagtacgcagcaatgtgctgaatcgttcgcacgaaaattggcattattgaaaaccaatgccaataatcatatccataaatatgtgtggttatgaactgcaag

SEQ ID NO: 16 - RNA, 114 nt, synthetic construct
  source 1..114: mol_type = other RNA
ggtgcgaataggaagtacgcagcaatgtgctgaatcgttcgcacgaaaattggcattattgaaaaccaatgccaataatcatatccataaatatgtgtggttatgaactgcaag

SEQ ID NO: 17 - RNA, 112 nt, synthetic construct
  source 1..112: mol_type = other RNA
tgcgaataggaagtacgcagcaatgtgctgaatcgttcgcacgaaaattggcattattgaaaaccaatgccaataatcatatccataaatatgtgtggttatgaactgcaag

SEQ ID NO: 18 - RNA, 111 nt, synthetic construct
  source 1..111: mol_type = other RNA
gcgaataggaagtacgcagcaatgtgctgaatcgttcgcacgaaaattggcattattgaaaaccaatgccaataatcatatccataaatatgtgtggttatgaactgcaag

SEQ ID NO: 19 - RNA, 130 nt, synthetic construct
  source 1..130: mol_type = other RNA
gtgaggtgcagccacgaggtgcgaataggaagtacgcagcgaaagctgaatcgttcgcacgaaaattggcattattgaaaaccaatgccaataatcatatccataaatatgtgtggttatgaactgcaag

SEQ ID NO: 20 - RNA, 129 nt, synthetic construct
  source 1..129: mol_type = other RNA
gtgaggtgcagccacgaggtgcgaatgaagtacgcagcaatgtgctgaatcgttcgcacgaaaattggcattattgaaaaccaatgccaataatcatatccataaatatgtgtggttatgaactgcaag

SEQ ID NO: 21 - RNA, 133 nt, synthetic construct
  source 1..133: mol_type = other RNA
gtgaggtgcagccacgaggtgcgaataggaagtacgcagcaatgtgctggaaatcgttcgcacgaaaattggcattattgaaaaccaatgccaataatcatatccataaatatgtgtggttatgaactgcaag

SEQ ID NO: 22 - RNA, 129 nt, synthetic construct
  source 1..129: mol_type = other RNA
gtgaggtgcagccacgaggtgcgaataggaagtacgcagaatgtctgaatcgttcgcacgaaaattggcattattgaaaaccaatgccaataatcatatccataaatatgtgtggttatgaactgcaag

SEQ ID NO: 23 - RNA, 127 nt, synthetic construct
  source 1..127: mol_type = other RNA
gtgaggtgcagccacgaggtgcgaataggaagtacgcgaatgtcgaatcgttcgcacgaaaattggcattattgaaaaccaatgccaataatcatatccataaatatgtgtggttatgaactgcaag

SEQ ID NO: 24 - RNA, 114 nt, synthetic construct
  source 1..114: mol_type = other RNA
gtgaggtgcagccacgaggtgcgaataggagaaatcgttcgcacgaaaattggcattattgaaaaccaatgccaataatcatatccataaatatgtgtggttatgaactgcaag

SEQ ID NO: 25 - RNA, 129 nt, synthetic construct
  source 1..129: mol_type = other RNA
gtgaggtgcagccacgaggtgcgataggaagtacgcagcaatgtgctgaatcgtcgcacgaaaattggcattattgaaaaccaatgccaataatcatatccataaatatgtgtggttatgaactgcaag

SEQ ID NO: 26 - RNA, 127 nt, synthetic construct
  source 1..127: mol_type = other RNA
gtgaggtgcagccacgaggtgcgtaggaagtacgcagcaatgtgctgaatcgcgcacgaaaattggcattattgaaaaccaatgccaataatcatatccataaatatgtgtggttatgaactgcaag

SEQ ID NO: 27 - RNA, 129 nt, synthetic construct
  source 1..129: mol_type = other RNA
gtgaggtgcagccacgaggtgcgaataggagtacgcagcaatgtgctgaacgttcgcacgaaaattggcattattgaaaaccaatgccaataatcatatccataaatatgtgtggttatgaactgcaag

SEQ ID NO: 28 - RNA, 123 nt, synthetic construct
  source 1..123: mol_type = other RNA
gtgaggtgcagccacgaggtgcgaaagtacgcagcaatgtgctgaattcgcacgaaaattggcattattgaaaaccaatgccaataatcatatccataaatatgtgtggttatgaactgcaag

SEQ ID NO: 29 - RNA, 133 nt, synthetic construct
  source 1..133: mol_type = other RNA
gtgaggtgcagccacgagggtgcgaatctaaagtggacagaaatgggaacagacgttcgcaccgaaaattggcattattgaaaaccaatgccaataatcatatccataaatatgtgtggttatgaactgcaag

SEQ ID NO: 30 - RNA, 130 nt, synthetic construct
  source 1..130: mol_type = other RNA
gtgaggtgcagccacgaggtgcgaataggaagtacgcagcaatgtgctgaatcgttcgcacgaaaattggcattattggaaacaatgccaataatcatatccataaatatgtgtggttatgaactgcaag

SEQ ID NO: 31 - RNA, 129 nt, synthetic construct
  source 1..129: mol_type = other RNA
gtgaggtgcagccacgaggtgcgaataggaagtacgcagcaatgtgctgaatcgttcgcacgaaaattggcattatgaaaaccatgccaataatcatatccataaatatgtgtggttatgaactgcaag

SEQ ID NO: 32 - RNA, 127 nt, synthetic construct
  source 1..127: mol_type = other RNA
gtgaggtgcagccacgaggtgcgaataggaagtacgcagcaatgtgctgaatcgttcgcacgaaaattggcattagaaaacctgccaataatcatatccataaatatgtgtggttatgaactgcaag

SEQ ID NO: 33 - RNA, 124 nt, synthetic construct
  source 1..124: mol_type = other RNA
gtgaggtgcagccacgaggtgcgaataggaagtacgcagcaatgtgctgaatcgttcgcacgaaaattggcagaaaacctgccaataatcatatccataaatatgtgtggttatgaactgcaag

SEQ ID NO: 34 - RNA, 129 nt, synthetic construct
  source 1..129: mol_type = other RNA
gtgaggtgcagccacgaggtgcgaataggaagtacgcagcaatgtgctgaatcgttcgcacgaaaattggattattgaaaaccaatccaataatcatatccataaatatgtgtggttatgaactgcaag

SEQ ID NO: 35 - RNA, 134 nt, synthetic construct
  source 1..134: mol_type = other RNA
gtgaggtgcagccacgaggtgcgaataggaagtacgcagcaatgtgctgaatcgttcgcacgaaagttggcggatttcttcggaaatcgagccaataatcatatccataaatatgtgtggttatgaactgcaag

SEQ ID NO: 36 - RNA, 134 nt, synthetic construct
  source 1..134: mol_type = other RNA
gtgaggtgcagccacgaggtgcgaataggaagtacgcagcaatgtgctgaatcgttcgcacgaaagttggcggatttcttcggaaatcgagccaacaatcatatccataaatatgtgtggttatgaactgcaag

SEQ ID NO: 37 - RNA, 129 nt, synthetic construct
  source 1..129: mol_type = other RNA
gtgaggtgcagccacgaggtgcgaataggaagtacgcagcaatgtgctgaatcgttcgcacgaaaattggcattattgaaaaccaatgccaataatcatatccatagaaatgtggttatgaactgcaag

SEQ ID NO: 38 - RNA, 129 nt, synthetic construct
  source 1..129: mol_type = other RNA
gtgaggtgcagccacgaggtgcgaataggaagtacgcagcaatgtgctgaatcgttcgcacgaaaattggcattattgaaaaccaatgccaataatcatatccaaaatatgttggttatgaactgcaag

SEQ ID NO: 39 - RNA, 127 nt, synthetic construct
  source 1..127: mol_type = other RNA
gtgaggtgcagccacgaggtgcgaataggaagtacgcagcaatgtgctgaatcgttcgcacgaaaattggcattattgaaaaccaatgccaataatcatatccaaatatgtggttatgaactgcaag

SEQ ID NO: 40 - RNA, 125 nt, synthetic construct
  source 1..125: mol_type = other RNA
gtgaggtgcagccacgaggtgcgaataggaagtacgcagcaatgtgctgaatcgttcgcacgaaaattggcattattgaaaaccaatgccaataatcatatcaaatatgtgttatgaactgcaag

SEQ ID NO: 41 - RNA, 123 nt, synthetic construct
  source 1..123: mol_type = other RNA
gtgaggtgcagccacgaggtgcgaataggaagtacgcagcaatgtgctgaatcgttcgcacgaaaattggcattattgaaaaccaatgccaataatcatataaatatgtttatgaactgcaag

SEQ ID NO: 42 - RNA, 121 nt, synthetic construct
  source 1..121: mol_type = other RNA
gtgaggtgcagccacgaggtgcgaataggaagtacgcagcaatgtgctgaatcgttcgcacgaaaattggcattattgaaaaccaatgccaataatcataaaatatgttatgaactgcaag

SEQ ID NO: 43 - RNA, 119 nt, synthetic construct
  source 1..119: mol_type = other RNA
gtgaggtgcagccacgaggtgcgaataggaagtacgcagcaatgtgctgaatcgttcgcacgaaaattggcattattgaaaaccaatgccaataatcataaatatgtatgaactgcaag

SEQ ID NO: 44 - RNA, 117 nt, synthetic construct
  source 1..117: mol_type = other RNA
gtgaggtgcagccacgaggtgcgaataggaagtacgcagcaatgtgctgaatcgttcgcacgaaaattggcattattgaaaaccaatgccaataatcaaaatatgttgaactgcaag

SEQ ID NO: 45 - RNA, 115 nt, synthetic construct
  source 1..115: mol_type = other RNA
gtgaggtgcagccacgaggtgcgaataggaagtacgcagcaatgtgctgaatcgttcgcacgaaaattggcattattgaaaaccaatgccaataatcaaatatgtgaactgcaag

SEQ ID NO: 46 - RNA, 134 nt, synthetic construct
  source 1..134: mol_type = other RNA
gtgaggtgcagccacgaggtgcgaataggaagtacgcagcaatgtgctgaatcgttcgcacgaaaattggcattattgaaaaccaatgccaataatcatatccatattatctgcggatggatgtgaactgcaag

SEQ ID NO: 47 - RNA, 134 nt, synthetic construct
  source 1..134: mol_type = other RNA
gtgaggtgcagccacgaggtgcgaataggaagtacgcagcaatgtgctgaatcgttcgcacgaaaattggcattattgaaaaccaatgccaataagcatatccatattatctgcggatggatgtgcactgcaag

SEQ ID NO: 48 - RNA, 131 nt, synthetic construct
  source 1..131: mol_type = other RNA
gtgaggtgcagccacgaggtgcgactaggaagtacgcagcaatgtgctgaatcggtcgcacgaaaattggcattattgaaaaccaatgccaataatcatatccataaatatgtgtggttatgaactgcaag

SEQ ID NO: 49 - RNA, 131 nt, synthetic construct
  source 1..131: mol_type = other RNA
gtgaggtgcagccacgaggtgcgaacaggaagtacgcagcaatgtgctgaatcgttcgcacgaaaattggcattattgaaaaccaatgccaataatcatatccataaatatgtgtggttatgaactgcaag

SEQ ID NO: 50 - RNA, 131 nt, synthetic construct
  source 1..131: mol_type = other RNA
gtgaggtgcagccacgaggtgcgaataggcagtacgcagcaatgtgctgaagcgttcgcacgaaaattggcattattgaaaaccaatgccaataatcatatccataaatatgtgtggttatgaactgcaag

SEQ ID NO: 51 - RNA, 131 nt, synthetic construct
  source 1..131: mol_type = other RNA
gtgaggtgcagccacgaggtgcgaataggaagtacgccgcaatgtgcggaatcgttcgcacgaaaattggcattattgaaaaccaatgccaataatcatatccataaatatgtgtggttatgaactgcaag

SEQ ID NO: 52 - RNA, 131 nt, synthetic construct
  source 1..131: mol_type = other RNA
gtgaggtgcagccacgaggtgcgaataggaagtacgcagcaatgtgctgaatcgttcgcacgaaacttggcattattgaaaaccaatgccaagaatcatatccataaatatgtgtggttatgaactgcaag

SEQ ID NO: 53 - RNA, 131 nt, synthetic construct
  source 1..131: mol_type = other RNA
gtgaggtgcagccacgaggtgcgaataggaagtacgcagcaatgtgctgaatcgttcgcacgaaaactggcattattgaaaaccaatgccagtaatcatatccataaatatgtgtggttatgaactgcaag

SEQ ID NO: 54 - RNA, 131 nt, synthetic construct
  source 1..131: mol_type = other RNA
gtgaggtgcagccacgaggtgcgaataggaagtacgcagcaatgtgctgaatcgttcgcacgaaaatcggcattattgaaaaccaatgccgataatcatatccataaatatgtgtggttatgaactgcaag

SEQ ID NO: 55 - RNA, 131 nt, synthetic construct
  source 1..131: mol_type = other RNA
gtgaggtgcagccacgaggtgcgaataggaagtacgcagcaatgtgctgaatcgttcgcacgaaaattggcattcttgaaaaccaaggccaataatcatatccataaatatgtgtggttatgaactgcaag

SEQ ID NO: 56 - RNA, 131 nt, synthetic construct
  source 1..131: mol_type = other RNA
gtgaggtgcagccacgaggtgcgaataggaagtacgcagcaatgtgctgaatcgttcgcacgaaaattggcattactgaaaaccagtgccaataatcatatccataaatatgtgtggttatgaactgcaag

SEQ ID NO: 57 - RNA, 131 nt, synthetic construct
  source 1..131: mol_type = other RNA
gtgaggtgcagccacgaggtgcgaataggaagtacgcagcaatgtgctgaatcgttcgcacgaaaattggcattatcgaaaaccgatgccaataatcatatccataaatatgtgtggttatgaactgcaag

SEQ ID NO: 58 - RNA, 131 nt, synthetic construct
  source 1..131: mol_type = other RNA
gtgaggtgcagccacgaggtgcgaataggaagtacgcagcaatgtgctgaatcgttcgcacgaaaattggcattattgaaaaccaatgccaataaccatatccataaatatgtgtggttatggactgcaag

SEQ ID NO: 59 - RNA, 131 nt, synthetic construct
  source 1..131: mol_type = other RNA
gtgaggtgcagccacgaggtgcgaataggaagtacgcagcaatgtgctgaatcgttcgcacgaaaattggcattattgaaaaccaatgccaataatcctatccataaatatgtgtggttaggaactgcaag

SEQ ID NO: 60 - RNA, 131 nt, synthetic construct
  source 1..131: mol_type = other RNA
gtgaggtgcagccacgaggtgcgaataggaagtacgcagcaatgtgctgaatcgttcgcacgaaaattggcattattgaaaaccaatgccaataatcacatccataaatatgtgtggttgtgaactgcaag

SEQ ID NO: 61 - RNA, 131 nt, synthetic construct
  source 1..131: mol_type = other RNA
gtgaggtgcagccacgaggtgcgaataggaagtacgcagcaatgtgctgaatcgttcgcacgaaaattggcattattgaaaaccaatgccaataatcatctccataaatatgtgtggtgatgaactgcaag

SEQ ID NO: 62 - RNA, 131 nt, synthetic construct
  source 1..131: mol_type = other RNA
gtgaggtgcagccacgaggtgcgaataggaagtacgcagcaatgtgctgaatcgttcgcacgaaaattggcattattgaaaaccaatgccaataatcatatccataaatatgtgtggatatgaactgcaag

SEQ ID NO: 63 - RNA, 131 nt, synthetic construct
  source 1..131: mol_type = other RNA
gtgaggtgcagccacgaggtgcgaataggaagtacgcagcaatgtgctgaatcgttcgcacgaaaattggcattattgaaaaccaatgccaataatcatatccgtaaatatgtgcggttatgaactgcaag

SEQ ID NO: 64 - RNA, 131 nt, synthetic construct
  source 1..131: mol_type = other RNA
gtgaggtgcagccacgaggtgcgaataggaagtacgcagcaatgtgctgaatcgttcgcacgaaaattggcattattgaaaaccaatgccaataatcatatccacaaatatgtgtggttatgaactgcaag

SEQ ID NO: 65 - RNA, 131 nt, synthetic construct
  source 1..131: mol_type = other RNA
gtgaggtgcagccacgaggtgcgaataggaagtacgcagcaatgtgctgaatcgttcgcacgaaaattggcattattgaaaaccaatgccaataatcatatccatgaatatgcgtggttatgaactgcaag

SEQ ID NO: 66 - RNA, 132 nt, synthetic construct
  source 1..132: mol_type = other RNA
cgtgaggtgcagccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 67 - RNA, 133 nt, synthetic construct
  source 1..133: mol_type = other RNA
tcgtgaggtgcagccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 68 - RNA, 134 nt, synthetic construct
  source 1..134: mol_type = other RNA
ttcgtgaggtgcagccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 69 - RNA, 136 nt, synthetic construct
  source 1..136: mol_type = other RNA
atttcgtgaggtgcagccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 70 - RNA, 137 nt, synthetic construct
  source 1..137: mol_type = other RNA
tatttcgtgaggtgcagccacgaggtgcgaataggaagtacgcagcaatgtgctgaatcgttcgcacgaaaattggcattattgaaaaccaatgccaataatcatatccataaatatgtgtggttatgaactgcaag

SEQ ID NO: 71 - RNA, 131 nt, synthetic construct
  source 1..131: mol_type = other RNA
gtgaggtgcagccacgaggtgcgaataggaagtacgcagcaatgtgctgaatcgttcgcacgaaaattggcatttttgaaaaccaaagccaataatcatatccataaatatgtgtggttatgaactgcaag

SEQ ID NO: 72 - RNA, 76 nt, synthetic construct
  source 1..76: mol_type = other RNA
tgtcaggcctgctagtcagccacagtttggggaaagctgtgcagcctgtaacccccccaggagaagctgggaaaaa

SEQ ID NO: 73 - RNA, 73 nt, synthetic construct
  source 1..73: mol_type = other RNA
ccagtggtctagtggtagaatagtaccctgccacggtacagacccgggttcgattcccggctggaaataaaaa

SEQ ID NO: 74 - DNA, 20 bp, synthetic construct
  source 1..20: mol_type = other DNA
attatcagcaagtggctgca

SEQ ID NO: 75 - RNA, 239 nt, synthetic construct
  source 1..239: mol_type = other RNA
caccgccgtcatcaacattgcctttctgttgcccagtaaacttatcatacggagccaggctatataattagctacttctccgtgaggtgcagccacgaggtgcgaataggagtacgcagcaatgtgctgaacgttcgcacgaaacctggcattattgaaaaccaatgccaggaaccgtctccataaatatgtgtggtgacggactgcaagtgttgccctcataacttatcttttttgcg

SEQ ID NO: 76 - RNA, 239 nt, synthetic construct
  source 1..239: mol_type = other RNA
caccgtgaggatgtagtcacggattcattatcaggttctggctgcagggtgcctgatctgtgccagggttaagcatgctgcgtgaggtgcagccacgaggtgcgaataggagtacgcagcaatgtgctgaacgttcgcacgaaacctggcattattgaaaaccaatgccaggaaccgtctccataaatatgtgtggtgacggactgcaagattatcagcaagtggctgcattttttgcg

SEQ ID NO: 77 - RNA, 315 nt, synthetic construct
  source 1..315: mol_type = other RNA
caccgtgtcaggcctgctagtcagccacagtttggggaaagctgtgcagcctgtaacccccccaggagaagctgggaaaaaccgtcatcaacattgcctttctgttgcccagtaaacttatcatacggagccaggctatataattagctacttctccgtgaggtgcagccacgaggtgcgaataggagtacgcagcaatgtgctgaacgttcgcacgaaacctggcattattgaaaaccaatgccaggaaccgtctccataaatatgtgtggtgacggactgcaagtgttgccctcataacttatcttttttgcg

SEQ ID NO: 78 - RNA, 315 nt, synthetic construct
  source 1..315: mol_type = other RNA
caccgtgtcaggcctgctagtcagccacagtttggggaaagctgtgcagcctgtaacccccccaggagaagctgggaaaaatgaggatgtagtcacggattcattatcaggttctggctgcagggtgcctgatctgtgccagggttaagcatgctgcgtgaggtgcagccacgaggtgcgaataggagtacgcagcaatgtgctgaacgttcgcacgaaacctggcattattgaaaaccaatgccaggaaccgtctccataaatatgtgtggtgacggactgcaagattatcagcaagtggctgcattttttgcg

SEQ ID NO: 79 - RNA, 312 nt, synthetic construct
  source 1..312: mol_type = other RNA
caccgccagtggtctagtggtagaatagtaccctgccacggtacagacccgggttcgattcccggctggaaataaaaaccgtcatcaacattgcctttctgttgcccagtaaacttatcatacggagccaggctatataattagctacttctccgtgaggtgcagccacgaggtgcgaataggagtacgcagcaatgtgctgaacgttcgcacgaaacctggcattattgaaaaccaatgccaggaaccgtctccataaatatgtgtggtgacggactgcaagtgttgccctcataacttatcttttttgcg

SEQ ID NO: 80 - RNA, 312 nt, synthetic construct
  source 1..312: mol_type = other RNA
caccgccagtggtctagtggtagaatagtaccctgccacggtacagacccgggttcgattcccggctggaaataaaaatgaggatgtagtcacggattcattatcaggttctggctgcagggtgcctgatctgtgccagggttaagcatgctgcgtgaggtgcagccacgaggtgcgaataggagtacgcagcaatgtgctgaacgttcgcacgaaacctggcattattgaaaaccaatgccaggaaccgtctccataaatatgtgtggtgacggactgcaagattatcagcaagtggctgcattttttgcg

SEQ ID NO: 81 - RNA, 241 nt, synthetic construct
  source 1..241: mol_type = other RNA
caccgccgtcatcaacattgcctttctgttgcccagtaaacttatcatacggagccaggctatataattagctacttctcttcgtgaggtgcagccacgaggtgcgaataggagtacgcagcaatgtgctgaacgttcgcacgaaacctggcattattgaaaaccaatgccaggaaccgtctccataaatatgtgtggtgacggactgcaagtgttgccctcataacttatcttttttgcg

SEQ ID NO: 82 - RNA, 241 nt, synthetic construct
  source 1..241: mol_type = other RNA
caccgtgaggatgtagtcacggattcattatcaggttctggctgcagggtgcctgatctgtgccagggttaagcatgctgttcgtgaggtgcagccacgaggtgcgaataggagtacgcagcaatgtgctgaacgttcgcacgaaacctggcattattgaaaaccaatgccaggaaccgtctccataaatatgtgtggtgacggactgcaagattatcagcaagtggctgcattttttgcg

SEQ ID NO: 83 - RNA, 317 nt, synthetic construct
  source 1..317: mol_type = other RNA
caccgtgtcaggcctgctagtcagccacagtttggggaaagctgtgcagcctgtaacccccccaggagaagctgggaaaaaccgtcatcaacattgcctttctgttgcccagtaaacttatcatacggagccaggctatataattagctacttctcttcgtgaggtgcagccacgaggtgcgaataggagtacgcagcaatgtgctgaacgttcgcacgaaacctggcattattgaaaaccaatgccaggaaccgtctccataaatatgtgtggtgacggactgcaagtgttgccctcataacttatcttttttgcg

SEQ ID NO: 84 - RNA, 317 nt, synthetic construct
  source 1..317: mol_type = other RNA
caccgtgtcaggcctgctagtcagccacagtttggggaaagctgtgcagcctgtaacccccccaggagaagctgggaaaaatgaggatgtagtcacggattcattatcaggttctggctgcagggtgcctgatctgtgccagggttaagcatgctgttcgtgaggtgcagccacgaggtgcgaataggagtacgcagcaatgtgctgaacgttcgcacgaaacctggcattattgaaaaccaatgccaggaaccgtctccataaatatgtgtggtgacggactgcaagattatcagcaagtggctgcattttttgcg

SEQ ID NO: 85 - RNA, 314 nt, synthetic construct
  source 1..314: mol_type = other RNA
caccgccagtggtctagtggtagaatagtaccctgccacggtacagacccgggttcgattcccggctggaaataaaaaccgtcatcaacattgcctttctgttgcccagtaaacttatcatacggagccaggctatataattagctacttctcttcgtgaggtgcagccacgaggtgcgaataggagtacgcagcaatgtgctgaacgttcgcacgaaacctggcattattgaaaaccaatgccaggaaccgtctccataaatatgtgtggtgacggactgcaagtgttgccctcataacttatcttttttgcg

SEQ ID NO: 86 - RNA, 314 nt, synthetic construct
  source 1..314: mol_type = other RNA
caccgccagtggtctagtggtagaatagtaccctgccacggtacagacccgggttcgattcccggctggaaataaaaatgaggatgtagtcacggattcattatcaggttctggctgcagggtgcctgatctgtgccagggttaagcatgctgttcgtgaggtgcagccacgaggtgcgaataggagtacgcagcaatgtgctgaacgttcgcacgaaacctggcattattgaaaaccaatgccaggaaccgtctccataaatatgtgtggtgacggactgcaagattatcagcaagtggctgcattttttgcg

SEQ ID NO: 87 - RNA, 226 nt, synthetic construct
  source 1..226: mol_type = other RNA
caccgccgtcatcaacattgcctttctgttgcccagtaaacttatcatacggagccaggctatataattagctacttctccgtgaggtgcagccacgaggtgcgaaagtacgcagcaatgtgctgaattcgcacgaaaattggcagaaaacctgccaataaccatctccataaatatgtgtggtgatggactgcaagtgttgccctcataacttatcttttttgcg

SEQ ID NO: 88 - RNA, 226 nt, synthetic construct
  source 1..226: mol_type = other RNA
caccgtgaggatgtagtcacggattcattatcaggttctggctgcagggtgcctgatctgtgccagggttaagcatgctgcgtgaggtgcagccacgaggtgcgaaagtacgcagcaatgtgctgaattcgcacgaaaattggcagaaaacctgccaataaccatctccataaatatgtgtggtgatggactgcaagattatcagcaagtggctgcattttttgcg

SEQ ID NO: 89 - RNA, 302 nt, synthetic construct
  source 1..302: mol_type = other RNA
caccgtgtcaggcctgctagtcagccacagtttggggaaagctgtgcagcctgtaacccccccaggagaagctgggaaaaaccgtcatcaacattgcctttctgttgcccagtaaacttatcatacggagccaggctatataattagctacttctccgtgaggtgcagccacgaggtgcgaaagtacgcagcaatgtgctgaattcgcacgaaaattggcagaaaacctgccaataaccatctccataaatatgtgtggtgatggactgcaagtgttgccctcataacttatcttttttgcg

SEQ ID NO: 90 - RNA, 302 nt, synthetic construct
  source 1..302: mol_type = other RNA
caccgtgtcaggcctgctagtcagccacagtttggggaaagctgtgcagcctgtaacccccccaggagaagctgggaaaaatgaggatgtagtcacggattcattatcaggttctggctgcagggtgcctgatctgtgccagggttaagcatgctgcgtgaggtgcagccacgaggtgcgaaagtacgcagcaatgtgctgaattcgcacgaaaattggcagaaaacctgccaataaccatctccataaatatgtgtggtgatggactgcaagattatcagcaagtggctgcattttttgcg

SEQ ID NO: 91 - RNA, 299 nt, synthetic construct
  source 1..299: mol_type = other RNA
caccgccagtggtctagtggtagaatagtaccctgccacggtacagacccgggttcgattcccggctggaaataaaaaccgtcatcaacattgcctttctgttgcccagtaaacttatcatacggagccaggctatataattagctacttctccgtgaggtgcagccacgaggtgcgaaagtacgcagcaatgtgctgaattcgcacgaaaattggcagaaaacctgccaataaccatctccataaatatgtgtggtgatggactgcaagtgttgccctcataacttatcttttttgcg

SEQ ID NO: 92 - RNA, 299 nt, synthetic construct
  source 1..299: mol_type = other RNA
caccgccagtggtctagtggtagaatagtaccctgccacggtacagacccgggttcgattcccggctggaaataaaaatgaggatgtagtcacggattcattatcaggttctggctgcagggtgcctgatctgtgccagggttaagcatgctgcgtgaggtgcagccacgaggtgcgaaagtacgcagcaatgtgctgaattcgcacgaaaattggcagaaaacctgccaataaccatctccataaatatgtgtggtgatggactgcaagattatcagcaagtggctgcattttttgcg

SEQ ID NO: 93 - RNA, 235 nt, synthetic construct
  source 1..235: mol_type = other RNA
caccgccgtcatcaacattgcctttctgttgcccagtaaacttatcatacggagccaggctatataattagctacttctcgtgaggtgcagccacgaggtgcgaaagtacgccgcaatgtgcggaattcgcacgaaagttggcggatttcttcggaaatcgagccaataaccatctccataaatatgtgtggtgatggactgcaagtgttgccctcataacttatcttttttgcg

SEQ ID NO: 94 - RNA, 235 nt, synthetic construct
  source 1..235: mol_type = other RNA
caccgtgaggatgtagtcacggattcattatcaggttctggctgcagggtgcctgatctgtgccagggttaagcatgctggtgaggtgcagccacgaggtgcgaaagtacgccgcaatgtgcggaattcgcacgaaagttggcggatttcttcggaaatcgagccaataaccatctccataaatatgtgtggtgatggactgcaagattatcagcaagtggctgcattttttgcg

SEQ ID NO: 95 - RNA, 311 nt, synthetic construct
  source 1..311: mol_type = other RNA
caccgtgtcaggcctgctagtcagccacagtttggggaaagctgtgcagcctgtaacccccccaggagaagctgggaaaaaccgtcatcaacattgcctttctgttgcccagtaaacttatcatacggagccaggctatataattagctacttctcgtgaggtgcagccacgaggtgcgaaagtacgccgcaatgtgcggaattcgcacgaaagttggcggatttcttcggaaatcgagccaataaccatctccataaatatgtgtggtgatggactgcaagtgttgccctcataacttatcttttttgcg

SEQ ID NO: 96 - RNA, 311 nt, synthetic construct
  source 1..311: mol_type = other RNA
caccgtgtcaggcctgctagtcagccacagtttggggaaagctgtgcagcctgtaacccccccaggagaagctgggaaaaatgaggatgtagtcacggattcattatcaggttctggctgcagggtgcctgatctgtgccagggttaagcatgctggtgaggtgcagccacgaggtgcgaaagtacgccgcaatgtgcggaattcgcacgaaagttggcggatttcttcggaaatcgagccaataaccatctccataaatatgtgtggtgatggactgcaagattatcagcaagtggctgcattttttgcg

SEQ ID NO: 97 - RNA, 308 nt, synthetic construct
  source 1..308: mol_type = other RNA
caccgccagtggtctagtggtagaatagtaccctgccacggtacagacccgggttcgattcccggctggaaataaaaaccgtcatcaacattgcctttctgttgcccagtaaacttatcatacggagccaggctatataattagctacttctcgtgaggtgcagccacgaggtgcgaaagtacgccgcaatgtgcggaattcgcacgaaagttggcggatttcttcggaaatcgagccaataaccatctccataaatatgtgtggtgatggactgcaagtgttgccctcataacttatcttttttgcg

SEQ ID NO: 98 - RNA, 308 nt, synthetic construct
  source 1..308: mol_type = other RNA
caccgccagtggtctagtggtagaatagtaccctgccacggtacagacccgggttcgattcccggctggaaataaaaatgaggatgtagtcacggattcattatcaggttctggctgcagggtgcctgatctgtgccagggttaagcatgctggtgaggtgcagccacgaggtgcgaaagtacgccgcaatgtgcggaattcgcacgaaagttggcggatttcttcggaaatcgagccaataaccatctccataaatatgtgtggtgatggactgcaagattatcagcaagtggctgcattttttgcg

SEQ ID NO: 99 - RNA, 231 nt, synthetic construct
  source 1..231: mol_type = other RNA
caccgccgtcatcaacattgcctttctgttgcccagtaaacttatcatacggagccaggctatataattagctacttctccgtgaggtgcagccacgaggtgcgaaagtacgccgcaatgtgcggaattcgcacgaaacttggcattattgaaaaccaatgccaagaatcctctccaaaatatgttggtgaggaactgcaagtgttgccctcataacttatcttttttgcg

SEQ ID NO: 100 - RNA, 231 nt, synthetic construct
  source 1..231: mol_type = other RNA
caccgtgaggatgtagtcacggattcattatcaggttctggctgcagggtgcctgatctgtgccagggttaagcatgctgcgtgaggtgcagccacgaggtgcgaaagtacgccgcaatgtgcggaattcgcacgaaacttggcattattgaaaaccaatgccaagaatcctctccaaaatatgttggtgaggaactgcaagattatcagcaagtggctgcattttttgcg

SEQ ID NO: 101 - RNA, 307 nt, synthetic construct
  source 1..307: mol_type = other RNA
caccgtgtcaggcctgctagtcagccacagtttggggaaagctgtgcagcctgtaacccccccaggagaagctgggaaaaaccgtcatcaacattgcctttctgttgcccagtaaacttatcatacggagccaggctatataattagctacttctccgtgaggtgcagccacgaggtgcgaaagtacgccgcaatgtgcggaattcgcacgaaacttggcattattgaaaaccaatgccaagaatcctctccaaaatatgttggtgaggaactgcaagtgttgccctcataacttatcttttttgcg

SEQ ID NO: 102 - RNA, 307 nt, synthetic construct
  source 1..307: mol_type = other RNA
caccgtgtcaggcctgctagtcagccacagtttggggaaagctgtgcagcctgtaacccccccaggagaagctgggaaaaatgaggatgtagtcacggattcattatcaggttctggctgcagggtgcctgatctgtgccagggttaagcatgctgcgtgaggtgcagccacgaggtgcgaaagtacgccgcaatgtgcggaattcgcacgaaacttggcattattgaaaaccaatgccaagaatcctctccaaaatatgttggtgaggaactgcaagattatcagcaagtggctgcattttttgcg

SEQ ID NO: 103 - RNA, 304 nt, synthetic construct
  source 1..304: mol_type = other RNA
caccgccagtggtctagtggtagaatagtaccctgccacggtacagacccgggttcgattcccggctggaaataaaaaccgtcatcaacattgcctttctgttgcccagtaaacttatcatacggagccaggctatataattagctacttctccgtgaggtgcagccacgaggtgcgaaagtacgccgcaatgtgcggaattcgcacgaaacttggcattattgaaaaccaatgccaagaatcctctccaaaatatgttggtgaggaactgcaagtgttgccctcataacttatcttttttgcg

SEQ ID NO: 104 - RNA, 304 nt, synthetic construct
  source 1..304: mol_type = other RNA
caccgccagtggtctagtggtagaatagtaccctgccacggtacagacccgggttcgattcccggctggaaataaaaatgaggatgtagtcacggattcattatcaggttctggctgcagggtgcctgatctgtgccagggttaagcatgctgcgtgaggtgcagccacgaggtgcgaaagtacgccgcaatgtgcggaattcgcacgaaacttggcattattgaaaaccaatgccaagaatcctctccaaaatatgttggtgaggaactgcaagattatcagcaagtggctgcattttttgcg

SEQ ID NO: 105 - RNA, 235 nt, synthetic construct
  source 1..235: mol_type = other RNA
caccgccgtcatcaacattgcctttctgttgcccagtaaacttatcatacggagccaggctatataattagctacttctcgtgaggtgcagccacgaggtgcgaaagtacgccgcaatgtgcggaattcgcacgaaacttggcattattgaaaaccaatgccaagaagcatatccatattatctgcggatggatgtgcactgcaagtgttgccctcataacttatcttttttgcg

SEQ ID NO: 106 - RNA, 235 nt, synthetic construct
  source 1..235: mol_type = other RNA
caccgtgaggatgtagtcacggattcattatcaggttctggctgcagggtgcctgatctgtgccagggttaagcatgctggtgaggtgcagccacgaggtgcgaaagtacgccgcaatgtgcggaattcgcacgaaacttggcattattgaaaaccaatgccaagaagcatatccatattatctgcggatggatgtgcactgcaagattatcagcaagtggctgcattttttgcg

SEQ ID NO: 107 - RNA, 311 nt, synthetic construct
  source 1..311: mol_type = other RNA
caccgtgtcaggcctgctagtcagccacagtttggggaaagctgtgcagcctgtaacccccccaggagaagctgggaaaaaccgtcatcaacattgcctttctgttgcccagtaaacttatcatacggagccaggctatataattagctacttctcgtgaggtgcagccacgaggtgcgaaagtacgccgcaatgtgcggaattcgcacgaaacttggcattattgaaaaccaatgccaagaagcatatccatattatctgcggatggatgtgcactgcaagtgttgccctcataacttatcttttttgcg

SEQ ID NO: 108 - RNA, 311 nt, synthetic construct
  source 1..311: mol_type = other RNA
caccgtgtcaggcctgctagtcagccacagtttggggaaagctgtgcagcctgtaacccccccaggagaagctgggaaaaatgaggatgtagtcacggattcattatcaggttctggctgcagggtgcctgatctgtgccagggttaagcatgctggtgaggtgcagccacgaggtgcgaaagtacgccgcaatgtgcggaattcgcacgaaacttggcattattgaaaaccaatgccaagaagcatatccatattatctgcggatggatgtgcactgcaagattatcagcaagtggctgcattttttgcg

SEQ ID NO: 109 - RNA, 308 nt, synthetic construct
  source 1..308: mol_type = other RNA
caccgccagtggtctagtggtagaatagtaccctgccacggtacagacccgggttcgattcccggctggaaataaaaaccgtcatcaacattgcctttctgttgcccagtaaacttatcatacggagccaggctatataattagctacttctcgtgaggtgcagccacgaggtgcgaaagtacgccgcaatgtgcggaattcgcacgaaacttggcattattgaaaaccaatgccaagaagcatatccatattatctgcggatggatgtgcactgcaagtgttgccctcataacttatcttttttgcg

SEQ ID NO: 110 - RNA, 308 nt, synthetic construct
  source 1..308: mol_type = other RNA
caccgccagtggtctagtggtagaatagtaccctgccacggtacagacccgggttcgattcccggctggaaataaaaatgaggatgtagtcacggattcattatcaggttctggctgcagggtgcctgatctgtgccagggttaagcatgctggtgaggtgcagccacgaggtgcgaaagtacgccgcaatgtgcggaattcgcacgaaacttggcattattgaaaaccaatgccaagaagcatatccatattatctgcggatggatgtgcactgcaagattatcagcaagtggctgcattttttgcg

SEQ ID NO: 111 - RNA, 230 nt, synthetic construct
  source 1..230: mol_type = other RNA
caccgccgtcatcaacattgcctttctgttgcccagtaaacttatcatacggagccaggctatataattagctacttctccgtgaggtgcagccacgaggtgcgaataggagtacgccgcaatgtgcggaacgttcgcacgaaacttggcagaaaacctgccaagaatcctatccaaaatatgttggttaggaactgcaagtgttgccctcataacttatcttttttgcg

SEQ ID NO: 112 - RNA, 230 nt, synthetic construct
  source 1..230: mol_type = other RNA
caccgtgaggatgtagtcacggattcattatcaggttctggctgcagggtgcctgatctgtgccagggttaagcatgctgcgtgaggtgcagccacgaggtgcgaataggagtacgccgcaatgtgcggaacgttcgcacgaaacttggcagaaaacctgccaagaatcctatccaaaatatgttggttaggaactgcaagattatcagcaagtggctgcattttttgcg

SEQ ID NO: 113 - RNA, 306 nt, synthetic construct
  source 1..306: mol_type = other RNA
caccgtgtcaggcctgctagtcagccacagtttggggaaagctgtgcagcctgtaacccccccaggagaagctgggaaaaaccgtcatcaacattgcctttctgttgcccagtaaacttatcatacggagccaggctatataattagctacttctccgtgaggtgcagccacgaggtgcgaataggagtacgccgcaatgtgcggaacgttcgcacgaaacttggcagaaaacctgccaagaatcctatccaaaatatgttggttaggaactgcaagtgttgccctcataacttatcttttttgcg

SEQ ID NO: 114 - RNA, 306 nt, synthetic construct
  source 1..306: mol_type = other RNA
caccgtgtcaggcctgctagtcagccacagtttggggaaagctgtgcagcctgtaacccccccaggagaagctgggaaaaatgaggatgtagtcacggattcattatcaggttctggctgcagggtgcctgatctgtgccagggttaagcatgctgcgtgaggtgcagccacgaggtgcgaataggagtacgccgcaatgtgcggaacgttcgcacgaaacttggcagaaaacctgccaagaatcctatccaaaatatgttggttaggaactgcaagattatcagcaagtggctgcattttttgcg

SEQ ID NO: 115 - RNA, 303 nt, synthetic construct
  source 1..303: mol_type = other RNA
caccgccagtggtctagtggtagaatagtaccctgccacggtacagacccgggttcgattcccggctggaaataaaaaccgtcatcaacattgcctttctgttgcccagtaaacttatcatacggagccaggctatataattagctacttctccgtgaggtgcagccacgaggtgcgaataggagtacgccgcaatgtgcggaacgttcgcacgaaacttggcagaaaacctgccaagaatcctatccaaaatatgttggttaggaactgcaagtgttgccctcataacttatcttttttgcg

SEQ ID NO: 116 - RNA, 303 nt, synthetic construct
  source 1..303: mol_type = other RNA
caccgccagtggtctagtggtagaatagtaccctgccacggtacagacccgggttcgattcccggctggaaataaaaatgaggatgtagtcacggattcattatcaggttctggctgcagggtgcctgatctgtgccagggttaagcatgctgcgtgaggtgcagccacgaggtgcgaataggagtacgccgcaatgtgcggaacgttcgcacgaaacttggcagaaaacctgccaagaatcctatccaaaatatgttggttaggaactgcaagattatcagcaagtggctgcattttttgcg

SEQ ID NO: 117 - RNA, 239 nt, synthetic construct
  source 1..239: mol_type = other RNA
caccgccgtcatcaacattgcctttctgttgcccagtaaacttatcatacggagccaggctatataattagctacttctccgtgaggtgcagccacgaggtgcgaaagtacgccgcaatgtgcggaattcgcacgaaagttggcggatttcttcggaaatcgagccaataagcatatccatattatctgcggatggatgtgcactgcaagtgttgccctcataacttatcttttttgcg

SEQ ID NO: 118 - RNA, 239 nt, synthetic construct
  source 1..239: mol_type = other RNA
caccgtgaggatgtagtcacggattcattatcaggttctggctgcagggtgcctgatctgtgccagggttaagcatgctgcgtgaggtgcagccacgaggtgcgaaagtacgccgcaatgtgcggaattcgcacgaaagttggcggatttcttcggaaatcgagccaataagcatatccatattatctgcggatggatgtgcactgcaagattatcagcaagtggctgcattttttgcg

SEQ ID NO: 119 - RNA, 315 nt, synthetic construct
  source 1..315: mol_type = other RNA
caccgtgtcaggcctgctagtcagccacagtttggggaaagctgtgcagcctgtaacccccccaggagaagctgggaaaaaccgtcatcaacattgcctttctgttgcccagtaaacttatcatacggagccaggctatataattagctacttctccgtgaggtgcagccacgaggtgcgaaagtacgccgcaatgtgcggaattcgcacgaaagttggcggatttcttcggaaatcgagccaataagcatatccatattatctgcggatggatgtgcactgcaagtgttgccctcataacttatcttttttgcg

SEQ ID NO: 120 - RNA, 315 nt, synthetic construct
  source 1..315: mol_type = other RNA
caccgtgtcaggcctgctagtcagccacagtttggggaaagctgtgcagcctgtaacccccccaggagaagctgggaaaaatgaggatgtagtcacggattcattatcaggttctggctgcagggtgcctgatctgtgccagggttaagcatgctgcgtgaggtgcagccacgaggtgcgaaagtacgccgcaatgtgcggaattcgcacgaaagttggcggatttcttcggaaatcgagccaataagcatatccatattatctgcggatggatgtgcactgcaagattatcagcaagtggctgcattttttgcg

SEQ ID NO: 121 - RNA, 312 nt, synthetic construct
  source 1..312: mol_type = other RNA
caccgccagtggtctagtggtagaatagtaccctgccacggtacagacccgggttcgattcccggctggaaataaaaaccgtcatcaacattgcctttctgttgcccagtaaacttatcatacggagccaggctatataattagctacttctccgtgaggtgcagccacgaggtgcgaaagtacgccgcaatgtgcggaattcgcacgaaagttggcggatttcttcggaaatcgagccaataagcatatccatattatctgcggatggatgtgcactgcaagtgttgccctcataacttatcttttttgcg

SEQ ID NO: 122 - RNA, 312 nt, synthetic construct
  source 1..312: mol_type = other RNA
caccgccagtggtctagtggtagaatagtaccctgccacggtacagacccgggttcgattcccggctggaaataaaaatgaggatgtagtcacggattcattatcaggttctggctgcagggtgcctgatctgtgccagggttaagcatgctgcgtgaggtgcagccacgaggtgcgaaagtacgccgcaatgtgcggaattcgcacgaaagttggcggatttcttcggaaatcgagccaataagcatatccatattatctgcggatggatgtgcactgcaagattatcagcaagtggctgcattttttgcg

SEQ ID NO: 123 - RNA, 301 nt, synthetic construct
  source 1..301: mol_type = other RNA
tgtcaggcctgctagtcagccacagtttggggaaagctgtgcagcctgtaacccccccaggagaagctgggaaaaaccgtcatcaacattgcctttctgttgcccagtaaacttatcatacggagccaggctatataattagctacttctccgtgaggtgcagccacgaggtgcgaaagtacgcagcaatgtgctgaattcgcacgaaagttggcggatttcttcggaaatcgagccaataatcatatccatattatctgcggatggatgtgaactgcaagtgttgccctcataacttatc

SEQ ID NO: 124 - RNA, 301 nt, synthetic construct
  source 1..301: mol_type = other RNA
tgtcaggcctgctagtcagccacagtttggggaaagctgtgcagcctgtaacccccccaggagaagctgggaaaaatgaggatgtagtcacggattcattatcaggttctggctgcagggtgcctgatctgtgccagggttaagcatgctgcgtgaggtgcagccacgaggtgcgaaagtacgcagcaatgtgctgaattcgcacgaaagttggcggatttcttcggaaatcgagccaataatcatatccatattatctgcggatggatgtgaactgcaagattatcagcaagtggctgca